(19) United States
(12) Patent Application Publication
(10) Pub. No.: US 2007/0117124 A1
(43) Pub. Date: May 24, 2007

(54) Achaete-Scute Like-2 Polypeptides and Encoding Nucleic Acids and Methods for the Diagnosis and Treatment of Tumor

(75) Inventors: Daryl Baldwin, San Francisco, CA (US); Hilary Clark, San Francisco, CA (US); Adrian Jubb, San Francisco, CA (US); Hartmut Koeppen, Berkeley, CA (US); Clifford Quan, Belmont, CA (US); Thomas Wu, San Francisco, CA (US); Zemin Zhang, Foster City, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/537,516

(22) Filed: Sep. 29, 2006

Related U.S. Application Data
(63) Continuation of application No. 10/454,945, filed on Jun. 4, 2003.
(63) Continuation of application No. PCT/US03/17682, filed on Jun. 4, 2003.
(60) Provisional application No. 60/407,087, filed on Aug. 29, 2002.
(60) Provisional application No. 60/407,087, filed on Aug. 29, 2002.

(51) Int. Cl. C12Q 1/68; C07H 21/04; C12P 21/06; C07K 14/82; C12N 1/21; C12N 1/18; C12N 5/06

(52) U.S. Cl. 435/6; 435/69.1; 435/320.1; 435/358; 435/252.33; 435/254.1; 530/350; 536/23.5

(57) ABSTRACT

The present invention is directed to compositions of matter useful for the diagnosis and treatment of tumor in mammals and to methods of using those compositions of matter for the same.

### RELATED APPLICATIONS

This application is a continuation of, and claims priority under 35 USC § 120 to both, PCT Application No. PCT/US2003/017682, filed Jun. 4, 2003, and also to U.S. application Ser. No. 10/454,945, filed Jun. 4, 2003, wherein PCT/US2003/017683, filed Jun. 4, 2003 and also U.S. application Ser. No. 10/454,945, filed Jun. 4, 2003, claims priority under 35 U.S.C. 119, to provisional application No. 60/407,087 filed Aug. 29, 2002.

### FIELD OF THE INVENTION

The present invention is directed to compositions of matter useful for the diagnosis and treatment of tumor in mammals and to methods of using those compositions of matter for the same.

### BACKGROUND OF THE INVENTION

Malignant tumors (cancers) are the second leading cause of death in the United States, after heart disease (Boring et al., CA Cancel J. Clin. 43:7 (1993)). Cancer is characterized by the increase in the number of abnormal, or neoplastic, cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites via a process called metastasis. In a cancerous state, a cell proliferates under conditions in which normal cells would not grow. Cancer manifests itself in a wide variety of forms, characterized by different degrees of invasiveness and aggressiveness.

In attempts to discover effective cellular targets for cancer diagnosis and therapy, researchers have sought to identify transmembrane or otherwise membrane-associated polypeptides that are specifically expressed on the surface of one or more particular type(s) of cancer cell as compared to on one or more normal non-cancerous cell(s). Often, such membrane-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via antibody-based therapies. In this regard, it is noted that antibody-based therapy has proved very effective in the treatment of certain cancers. For example, HERCEPTIN® and RITUXAN® (both from Genentech Inc., South San Francisco, Calif.) are antibodies that have been used successfully to treat breast cancer and non-Hodgkin's lymphoma, respectively. More specifically, HERCEPTIN® is a recombinant DNA-derived humanized monoclonal antibody that selectively binds to the extracellular domain of the human epidermal growth factor receptor 2 (HER2) proto-oncogene. HER2 protein overexpression is observed in 25-30% of primary breast cancers. RITUXAN® is a genetically engineered chimeric murine/human monoclonal antibody directed against the CD20 antigen found on the surface of normal and malignant B lymphocytes. Both these antibodies are recombinantly produced in CHO cells.

In other attempts to discover effective cellular targets for cancer diagnosis and therapy, researchers have sought to identify (1) non-membrane-associated polypeptides that are specifically produced by one or more particular type(s) of cancer cell(s) as compared to by one or more particular type(s) of non-cancerous normal cell(s), (2) polypeptides that are produced by cancer cells at an expression level that is significantly higher than that of one or more normal non-cancerous cell(s), or (3) polypeptides whose expression is specifically limited to only a single (or very limited number of different) tissue type(s) in both the cancerous and non-cancerous state (e.g., normal prostate and prostate tumor tissue). Such polypeptides may remain intracellularly located or may be secreted by the cancer cell. Moreover, such polypeptides may be expressed not by the cancer cell itself, but rather by cells which produce and/or secrete polypeptides having a potentiating or growth-enhancing effect on cancer cells. Such secreted polypeptides are often proteins that provide cancer cells with a growth advantage over normal cells and include such things as, for example, angiogenic factors, cellular adhesion factors, growth factors, and the like. Identification of antagonists of such non-membrane associated polypeptides would be expected to serve as effective therapeutic agents for the treatment of such cancers. Furthermore, identification of the expression pattern of such polypeptides would be useful for the diagnosis of particular cancers in mammals.

Despite the above identified advances in mammalian cancer therapy, there is a great need for additional diagnostic and therapeutic agents capable of detecting the presence of tumor in a mammal and for effectively inhibiting neoplastic cell growth, respectively. Accordingly, it is an objective of the present invention to identify: (1) cell membrane-associated polypeptides that are more abundantly expressed on one or more type(s) of cancer cell(s) as compared to on normal cells or on other different cancer cells, (2) non-membrane-associated polypeptides that are specifically produced by one or more particular type(s) of cancer cell(s) (or by other cells that produce polypeptides having a potentiating effect on the growth of cancer cells) as compared to by one or more particular type(s) of non-cancerous normal cell(s), (3) non-membrane-associated polypeptides that are produced by cancer cells at an expression level that is significantly higher than that of one or more normal non-cancerous cell(s), or (4) polypeptides whose expression is specifically limited to only a single (or very limited number of different) tissue type(s) in both a cancerous and non-cancerous state (e.g., normal prostate and prostate tumor tissue), and to use those polypeptides, and their encoding nucleic acids, to produce compositions of matter useful in the therapeutic treatment and diagnostic detection of cancer in mammals. It is also an objective of the present invention to identify cell membrane-associated, secreted or intracellular polypeptides whose expression is limited to a single or very limited number of tissues, and to use those polypeptides, and their encoding nucleic acids, to produce compositions of matter useful in the therapeutic treatment and diagnostic detection of cancer in mammals.

In particular, it is an objective of the present invention to identify effective cellular targets for cancer including colorectal cancer (or neoplasms) which will have a functional role in tumorigenesis. Colorectal cancer (CRC) confers significant morbidity and mortality on Western populations, which have an incidence approximately 1.5 fold higher than elsewhere in the world. Around 95% of CRCs arise sporadically, with the remainder clustering into recognized familial syndromes that predispose to tumors at an earlier age. The most prevalent are heriditary non-polyposis CRC, the various pleiotropic phenotypes of familial adenomatous polyposis, juvenile polyposis and Peutz-Jegher's syndrome (see Boland C. R., The Genetic Basis of Human Cancer. London: McGraw-Hill; 333-346 (1999); Foulkes, W. D.; QJM, 88 (12): 853-863 (1995); Hardy, R. G. et al; BMJ, 321 (7265): 886-889 (2000)). Similarly, the first-degree relatives of a patient diagnosed with sporadic CRC are at approximately twice the average risk. The favored treatment regime for operable disease relies on surgical excision of the primary lesion. Adjuvant chemotherapy with 5-fluorouracil and folinic acid has proven survival benefits for patients with Dukes' stage C tumors [O'Connell, M. J. et al., J Clin Oncol, 15(1):246-50 (1997)]. Local preoperative radiotherapy is also efficacious in prolonging the life of patients with rectal tumors. Nevertheless, the prognosis of CRC remains poor, prompting the search for novel drug targets through a better understanding of the molecular biology that underpins the disease. This rationale has been validated by the clinical success of Rituxan and Gleevec, which respectively target the CD20 antigen in non-Hodgkin's lymphoma and the bcr-abl tyrosine kinase in chronic myeloid leukemia (Countouriotis, A. et al, Stem Cells, 20(3):215-229 (2002)).

Adenocarcinomas account for 98% of all CRCs, and are believed to arise from stem-cells in the crypts of Lieberkhuns that have undergone several rounds of clonal selection. In the large-bowel, this is a multi-step process referred to as the “adenoma-to-adenocarcinoma” sequence (Muto, T. et al., Cancer, 36(6):2251-2270 (1975). Vogelstein proposed a model in which the progression of certain colonic neoplasms through the stages of the adenoma-to-adenocarcinoma sequence is driven by the successive acquisition of stereotyped genetic, epigenetic and/or karyotypic events (Kinzler, K. W. et al., The Genetic Basis of Human Cancer. London: McGraw-Hill: 565-587 (1999)). Although this is not representative of all CRCs, it illustrates some of the principal oncogenic and tumor suppressor pathways that define molecular subtypes of CRC. They include the wntl, epidermal growth factor receptor (EGFR), transforming growth factor (TGF)-β and p53 signal transduction pathways, in addition to defects in caretaker genes concerned with microsataellite and/or chromosomal instability [Jubb, A. M. et al., Ann Ny Acad Sci, 983:251-67 (2003); Lengauer, C. et al., Nature, 386(6625):623-7 (1997)]. Many of these pathways have also been implicated in embryogenesis, facilitating the dissection of signaling networks operating in CRC and the identification of potential drug targets. In this regard, a neurogenesis guiding complex known as the “achaete-scute gene complex” (ac-sc) has been identified in Drosophila melanogaster which is thought to be responsible for guiding neurogenesis (Villares, R. et al, Cell, 50(3):415-424 (1987)).

There are many orthologs of the vertebrate ac-sc family, including the achaete-scute homolog 1 (ASH1), which has been described in all species examined (e.g. human (H)ASH1, mammalian (M)ASH1 in rodents, chick (C)ASH1 in Gallus gallus, zebrafish (Z)ASH1a and (Z)ASH1b in Danio rerio and Xenopus (X)ASH1 in Xenopus laevis (Bertrand N. et al., Nat Rev Neurosci, 3(7):517-530 (2002)). Paralogues of these genes have been described, but each one is only present in a single class of vertebrates (for example HASH2/ASCL2 for the human paralogue and MASH2 for the murine paralogue). Products of ac-sc and its orthologues belong to a conserved family of transcriptional regulators defined by the presence of basic and helix-loop-helix (HLH) domains. These proteins function as dimers through their HLH domains, which permits the basic domains to bind E-box elements (CANNTG; SEQ ID NO:5) and control transcription in promoter and enhancer sequences. Ac-sc and its orthologues are defined as class II HLH proteins [Massari, M. E., et al., Mol Cell Biol, 20(2):429-40 (2000)]. With a few exceptions, they preferentially heterodimeriz with positive-regulatory class I HLH proteins or negative-regulatory class V HLH proteins.

MASH1 and MASH2 proteins function as lineage-specific transcription factors essential for development of the neurectoderm and trophetctoderm, respectively. MASH2 has also been observed in the schwann cells of adult peripheral nerves, where it appears to be a negative regulator of proliferation (Kury, P. et al., J Neurosci, 22(17):7586-7595 (2002)). Both MASH2 and HASH2 genes are maternally imprinted, and lie within an imprinting cluster on distal chromosome 7 and 11p15 respectively [Guillemot, F. et al, Nat Genet 9(3):235-242 (1995); Westerman, B. A. et al., Placenta 22(6):511-518 (2001); Miyamoto, T. et al., Cytogenet Cell Genet 73(4):312-314 (1996); Miyamota, T. et al, J Hum Genet 43(1):69-70 (1998); Miyamoto, T. et al, J Assist Reprod Genet 19(5):240-244 (2002)). The Beckwith-Wiedemann syndrome of fetal overgrowth and tumor development is associated with loss of imprinting at 11p15, which has also been observed in sporadic CRC, [Engemann, S. et al, Hum Mol Genet, 9(18):2691-706 (2000); Paulsen, M. et al, Hum Mol Genet, 7(7):1149-59 (1998); Feinberg, A. P., Cancer Res 59(7 Suppl):1743s-6s (1999); Ping, A, J., Am J Hum Genet, 44(5):720-3 (1989), Fleisher, A. S. et al., Gastroenterology, 118(3):637 (2000), Miyaki, M., Nat Med, 4(11):1236-7 (1998)]. Moreover, there is a case-report describing colonic polyposis in a patient with Beckwith-Wiedemann syndrome and concurrent aberrations of wnt1 signaling and loss of heterozygosity at 11p have been reported in cases of pancreatoblastoma [Kerr, N. J., et al., Am J Pathol 160(4):1541-2 (2002); Abraham, S. C., et al., Am J Pathol, 159(5):1619-27 (2001)]. HASH2 is also expressed in the extravillous trophoblast cell lineage of the early human placenta (Alders, M. et al., Hum Mol Genet, 6(6):859-867 (1997)). An unspliced variant of the mature HASH2 message is reported to contain an open reading frame for an uncharacterized protein, human achaete-scute associated protein (HASAP) (Westermann, B. A., et al., Placenta, 22(6):511-518 (2001)).

### SUMMARY OF THE INVENTION

### A. EMBODIMENTS

In the present specification, Applicants describe for the first time the identification of various cellular polypeptides (and their encoding nucleic acids or fragments thereof) which are expressed to a greater degree on the surface of or by one or more types of cancer cell(s) as compared to on the surface of or by one or more types of normal non-cancer cells. Alternatively, such polypeptides are expressed by cells which produce and/or secrete polypeptides having a potentiating or growth-enhancing effect on cancer cells. Again alternatively, such polypeptides may not be overexpressed by tumor cells as compared to normal cells of the same tissue type, but rather may be specifically expressed by both tumor cells and normal cells of only a single or very limited number of tissue types (preferably tissues which are not essential for life, e.g., prostate, etc.). All of the above polypeptides are herein referred to as Tumor-associated Antigenic Target polypeptides 376 and 377 (“TAT376” and “TAT377” respectively) and are expected to serve as effective targets for cancer therapy and diagnosis in mammals.

Accordingly, in one embodiment of the present invention, the invention provides an isolated nucleic acid molecule having a nucleotide sequence that encodes a tumor-associated antigenic target polypeptide or fragment thereof (a “TAT376” or “TAT377” polypeptide).

In certain aspects, the isolated nucleic acid molecule comprises a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% nucleic acid sequence identity, to (a) a DNA molecule encoding a full-length TAT376 or TAT377 polypeptide having an amino acid sequence as disclosed herein, a TAT376 or TAT377 polypeptide amino acid sequence lacking the signal peptide as disclosed herein, an extracellular domain of a transmembrane TAT376 or TAT377 polypeptide, with or without the signal peptide, as disclosed herein or any other specifically defined fragment of a full-length TAT376 or TAT377 polypeptide amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100nucleic acid sequence identity, to (a) a DNA molecule comprising the coding sequence of a full-length TAT376 or TAT377 polypeptide cDNA as disclosed herein, the coding sequence of a TAT376 or TAT377 polypeptide lacking the signal peptide as disclosed herein, the coding sequence of an extracellular domain of a transmembrane TAT376 or TAT377 polypeptide, with or without the signal peptide, as disclosed herein or the coding sequence of any other specifically defined fragment of the full-length TAT376 or TAT377 polypeptide amino acid sequence as disclosed herein, or (b) the complement of the DNA molecule of (a).

In further aspects, the invention concerns an isolated nucleic acid molecule comprising a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% nucleic acid sequence identity, to (a) a DNA molecule that encodes the same mature polypeptide encoded by the full-length coding region of any of the human protein cDNAs deposited with the ATCC as disclosed herein, or (b) the complement of the DNA molecule of (a).

Another aspect of the invention provides an isolated nucleic acid molecule comprising a nucleotide sequence encoding a TAT376 or TAT377 polypeptide which is either transmembrane domain-deleted or transmembrane domain-inactivated, or is complementary to such encoding nucleotide sequence, wherein the transmembrane domain(s) of such polypeptide(s) are disclosed herein. Therefore, soluble extracellular domains of the herein described TAT376 or TAT377 polypeptides are contemplated.

In other aspects, the present invention is directed to isolated nucleic acid molecules which hybridize to (a) a nucleotide sequence encoding a TAT376 or TAT377 polypeptide having a full-length amino acid sequence as disclosed herein, a TAT376 or TAT377 polypeptide amino acid sequence lacking the signal peptide as disclosed herein, an extracellular domain of a transmembrane TAT376 or TAT377 polypeptide, with or without the signal peptide, as disclosed herein or any other specifically defined fragment of a full-length TAT376 or TAT377 polypeptide amino acid sequence as disclosed herein, or (b) the complement of the nucleotide sequence of (a). In this regard, an embodiment of the present invention is directed to fragments of a full-length TAT376 or TAT377 polypeptide coding sequence, or the complement thereof, as disclosed herein, that may find use as, for example, hybridization probes useful as, for example, diagnostic probes, antisense oligonucleotide probes, or for encoding fragments of a full-length TAT376 or TAT377 polypeptide that may optionally encode a polypeptide comprising a binding site for an anti-TAT376 or anti-TAT377 polypeptide antibody, a TAT376 or TAT377 binding oligopeptide or other small organic molecule that binds to a TAT376 or TAT377 polypeptide. Such nucleic acid fragments are usually at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length. It is noted that novel fragments of a TAT376 or TAT377 polypeptide-encoding nucleotide sequence may be determined in a routine manner by aligning the TAT376 or TAT377 polypeptide-encoding nucleotide sequence with other known nucleotide sequences using any of a number of well known sequence alignment programs and determining which TAT376 or TAT377 polypeptide-encoding nucleotide sequence fragment(s) are novel. All of such novel fragments of TAT376 or TAT377 polypeptide-encoding nucleotide sequences are contemplated herein. Also contemplated are the TAT376 or TAT377 polypeptide fragments encoded by these nucleotide molecule fragments, preferably those TAT376 or TAT377 polypeptide fragments that comprise a binding site for an anti-TAT376 or anti-TAT377 antibody, a TAT376 or TAT377 binding oligopeptide or other small organic molecule that binds to a TAT376 or TAT377 polypeptide.

In another embodiment, the invention provides isolated TAT376 or TAT377 polypeptides encoded by any of the isolated nucleic acid sequences hereinabove identified.

In a certain aspect, the invention concerns an isolated TAT376 or TAT377 polypeptide, comprising an amino acid sequence having at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% amino acid sequence identity, to a TAT376 or TAT377 polypeptide having a full-length amino acid sequence as disclosed herein, a TAT376 or TAT377 polypeptide amino acid sequence lacking the signal peptide as disclosed herein, an extracellular domain of a transmembrane TAT376 or TAT377 polypeptide protein, with or without the signal peptide, as disclosed herein, an amino acid sequence encoded by any of the nucleic acid sequences disclosed herein or any other specifically defined fragment of a full-length TAT376 or TAT377 polypeptide amino acid sequence as disclosed herein.

In a further aspect, the invention concerns an isolated TAT376 or TAT377 polypeptide comprising an amino acid sequence having at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to an amino acid sequence encoded by any of the human protein cDNAs deposited with the ATCC as disclosed herein.

In a specific aspect, the invention provides an isolated TAT376 or TAT377 polypeptide. without the N-terminal signal sequence and/or without the initiating methionine and is encoded by a nucleotide sequence that encodes such an amino acid sequence as hereinbefore described. Processes for producing the same are also herein described, wherein those processes comprise culturing a host cell comprising a vector which comprises the appropriate encoding nucleic acid molecule under conditions suitable for expression of the TAT376 or TAT377 polypeptide and recovering the TAT376 or TAT377 polypeptide from the cell culture.

Another aspect of the invention provides an isolated TAT376 or TAT377 polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the TAT376 or TAT377 polypeptide and recovering the TAT376 or TAT377 polypeptide from the cell culture.

In other embodiments of the present invention, the invention provides vectors comprising DNA encoding any of the herein described polypeptides. Host cells comprising any such vector are also provided. By way of example, the host cells may be CHO cells, E. coli cells, or yeast cells. A process for producing any of the herein described polypeptides is further provided and comprises culturing host cells under conditions suitable for expression of the desired polypeptide and recovering the desired polypeptide from the cell culture.

In other embodiments, the invention provides isolated chimeric polypeptides comprising any of the herein described TAT376 or TAT377 polypeptides fused to a heterologous (non-TAT376 or non-TAT377) polypeptide. Example of such chimeric molecules comprise any of the herein described TAT376 or TAT377 polypeptides fused to a heterologous polypeptide such as, for example, an epitope tag sequence or a Fc region of an immunoglobulin.

In another embodiment, the invention provides an antibody which binds, preferably specifically, to any of the above or below described polypeptides. Optionally, the antibody is a monoclonal antibody, antibody fragment, chimeric antibody, humanized antibody, single-chain antibody or antibody that competitively inhibits the binding of an anti-TAT376 or anti-TAT377 polypeptide antibody to its respective antigenic epitope. Antibodies of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies of the present invention may optionally be produced in CHO cells or bacterial cells and preferably induce death of a cell to which they bind. For diagnostic purposes, the antibodies of the present invention may be detectably labeled, attached to a solid support, or the like.

In other embodiments of the present invention, the invention provides vectors comprising DNA encoding any of the herein described antibodies. Host cell comprising any such vector are also provided. By way of example, the host cells may be CHO cells, E. coli cells, or yeast cells. A process for producing any of the herein described antibodies is further provided and comprises culturing host cells under conditions suitable for expression of the desired antibody and recovering the desired antibody from the cell culture.

In another embodiment, the invention provides oligopeptides (“TAT376 or TAT377 binding oligopeptides”) which bind, preferably specifically, to any of the above or below described TAT376 or TAT377 polypeptides. Optionally, the TAT376 or TAT377 binding oligopeptides of the present invention may be conjugated to a growth inhibitory agent or cytotoxic agent such as a toxin, including, for example, a maytansinoid or calicheamicin, an antibiotic, a radioactive isotope, a nucleolytic enzyme, or the like. The TAT376 or TAT377 binding oligopeptides of the present invention may optionally be produced in CHO cells or bacterial cells and preferably induce death of a cell to which they bind. For diagnostic purposes, the TAT376 or TAT377 binding oligopeptides of the present invention may be detectably labeled, attached to a solid support, or the like.

In other embodiments of the present invention, the invention provides vectors comprising DNA encoding any of the herein described TAT376 or TAT377 binding oligopeptides. Host cell comprising any such vector are also provided. By way of example, the host cells may be CHO cells, E. coli cells, or yeast cells. A process for producing any of the herein described TAT376 or TAT377 binding oligopeptides is further provided and comprises culturing host cells under conditions suitable for expression of the desired oligopeptide and recovering the desired oligopeptide from the cell culture.

In another embodiment, the invention provides small organic molecules (“TAT376 or TAT377 binding organic molecules”) which bind, preferably specifically, to any of the above or below described TAT376 or TAT377 polypeptides. Optionally, the TAT376 or TAT377 binding organic molecules of the present invention may be conjugated to a growth inhibitory agent or cytotoxic agent such as a toxin, including, for example, a maytansinoid or calicheamicin, an antibiotic, a radioactive isotope, a nucleolytic enzyme, or the like. The TAT376 or TAT377 binding organic molecules of the present invention preferably induce death of a cell to which they bind. For diagnostic purposes, the TAT376 or TAT377 binding organic molecules of the present invention may be detectably labeled, attached to a solid support, or the like.

In a still further embodiment, the invention concerns a composition of matter comprising a TAT376 or TAT377 polypeptide as described herein, a chimeric TAT376 or TAT377 polypeptide as described herein, an anti-TAT376 or anti-TAT377 antibody as described herein, a TAT376 or TAT377 binding oligopeptide as described herein, or a TAT376 or TAT377 binding organic molecule as described herein, in combination with a carrier. Optionally, the carrier is a pharmaceutically acceptable carrier.

In yet another embodiment, the invention concerns an article of manufacture comprising a container and a composition of matter contained within the container, wherein the composition of matter may comprise a TAT376 or TAT377 polypeptide as described herein, a chimeric TAT376 or TAT377 polypeptide as described herein, an anti-TAT376 or anti-TAT377 antibody as described herein, a TAT376 or TAT377 binding oligopeptide as described herein, or a TAT376 or TAT377 binding organic molecule as described herein. The article may further optionally comprise a label affixed to the container, or a package insert included with the container, that refers to the use of the composition of matter for the therapeutic treatment or diagnostic detection of a tumor.

Another embodiment of the present invention is directed to the use of a TAT376 or TAT377 polypeptide as described herein, a chimeric TAT376 or TAT377 polypeptide as described herein, an anti-TAT376 or TAT377 polypeptide antibody as described herein, a TAT376 or TAT377 binding oligopeptide as described herein, or a TAT376 or TAT377 binding organic molecule as described herein, for the preparation of a medicament useful in the treatment of a condition which is responsive to the TAT376 or TAT377 polypeptide, chimeric TAT376 or TAT377 polypeptide, anti-TAT376 or anti-TAT377 polypeptide antibody, TAT376 or TAT377 binding oligopeptide, or TAT376 or TAT377 binding organic molecule.

### B. ADDITIONAL EMBODIMENTS

Another embodiment of the present invention is directed to a method for inhibiting the growth of a cell that expresses a TAT376 or TAT377 polypeptide, wherein the method comprises contacting the cell with an antibody, an oligopeptide or a small organic molecule that binds to the TAT376 or TAT377 polypeptide, and wherein the binding of the antibody, oligopeptide or organic molecule to the TAT376 or TAT377 polypeptide causes inhibition of the growth of the cell expressing the TAT376 or TAT377 polypeptide. In preferred embodiments, the cell is a cancer cell and binding of the antibody, oligopeptide or organic molecule to the TAT376 or TAT377 polypeptide causes death of the cell expressing the TAT376 or TAT377 polypeptide. Optionally, the antibody is a monoclonal antibody, antibody fragment, chimeric antibody, humanized antibody, or single-chain antibody. Antibodies, TAT376 or TAT377 binding oligopeptides and TAT376 or TAT377 binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and TAT376 or TAT377 binding oligopeptides employed in the methods of the present invention may optionally be produced in CHO cells or bacterial cells.

Yet another embodiment of the present invention is directed to a method of therapeutically treating a mammal having a cancerous tumor comprising cells that express a TAT376 or TAT377 polypeptide, wherein the method comprises administering to the mammal a therapeutically effective amount of an antibody, an oligopeptide or a small organic molecule that binds to the TAT376 or TAT377 polypeptide, thereby resulting in the effective therapeutic treatment of the tumor. Optionally, the antibody is a monoclonal antibody, antibody fragment, chimeric antibody, humanized antibody, or single-chain antibody. Antibodies, TAT376 or TAT377 binding oligopeptides and TAT376 or TAT377 binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and oligopeptides employed in the methods of the present invention may optionally be produced in CHO cells or bacterial cells.

Yet another embodiment of the present invention is directed to a method of determining the presence of a TAT376 or TAT377 polypeptide in a sample suspected of containing the TAT376 or TAT377 polypeptide, wherein the method comprises exposing the sample to an antibody, oligopeptide or small organic molecule that binds to the TAT376 or TAT377 polypeptide and determining binding of the antibody, oligopeptide or organic molecule to the TAT376 or TAT377 polypeptide in the sample, wherein the presence of such binding is indicative of the presence of the TAT376 or TAT377 polypeptide in the sample. Optionally, the sample may contain cells (which may be cancer cells) suspected of expressing the TAT376 or TAT377 polypeptide. The antibody, TAT376 or TAT377 binding oligopeptide or TAT376 or TAT377 binding organic molecule employed in the method may optionally be detectably labeled, attached to a solid support, or the like.

A further embodiment of the present invention is directed to a method of diagnosing the presence of a tumor in a mammal, wherein the method comprises detecting the level of expression of a gene encoding a TAT376 or TAT377 polypeptide (a) in a test sample of tissue cells obtained from said mammal, and (b) in a control sample of known normal non-cancerous cells of the same tissue origin or type, wherein a higher level of expression of the TAT376 or TAT377 polypeptide in the test sample, as compared to the control sample, is indicative of the presence of tumor in the mammal from which the test sample was obtained.

Another embodiment of the present invention is directed to a method of diagnosing the presence of a tumor in a mammal, wherein the method comprises (a) contacting a test sample comprising tissue cells obtained from the mammal with an antibody, oligopeptide or small organic molecule that binds to a TAT376 or TAT377 polypeptide and (b) detecting the formation of a complex between the antibody, oligopeptide or small organic molecule and the TAT376 or TAT377 polypeptide in the test sample, wherein the formation of a complex is indicative of the presence of a tumor in the mammal. Optionally, the antibody, TAT376 or TAT377 binding oligopeptide or TAT376 or TAT377 binding organic molecule employed is detectably labeled, attached to a solid support, or the like, and/or the test sample of tissue cells is obtained from an individual suspected of having a cancerous tumor.

Yet another embodiment of the present invention is directed to a method for treating or preventing a cell proliferative disorder associated with altered, preferably increased, expression or activity of a TAT376 or TAT377 polypeptide, the method comprising administering to a subject in need of such treatment an effective amount of an antagonist of a TAT376 or TAT377 polypeptide. Preferably, the cell proliferative disorder is cancer and the antagonist of the TAT376 or TAT377 polypeptide is an anti-TAT376 or anti-TAT377 polypeptide antibody, TAT376 or TAT377 binding oligopeptide, TAT376 or TAT377 binding organic molecule or antisense oligonucleotide. Effective treatment or prevention of the cell proliferative disorder may be a result of direct killing or growth inhibition of cells that express a TAT376 or TAT377 polypeptide or by antagonizing the cell growth potentiating activity of a TAT376 or TAT377 polypeptide.

Yet another embodiment of the present invention is directed to a method of binding an antibody, oligopeptide or small organic molecule to a cell that expresses a TAT376 or TAT377 polypeptide, wherein the method comprises contacting a cell that expresses a TAT376 or TAT377 polypeptide with said antibody, oligopeptide or small organic molecule under conditions which are suitable for binding of the antibody, oligopeptide or small organic molecule to said TAT376 or TAT377 polypeptide and allowing binding therebetween.

Other embodiments of the present invention are directed to the use of (a) a TAT376 or TAT377 polypeptide, (b) a nucleic acid encoding a TAT376 or TAT377 polypeptide or a vector or host cell comprising that nucleic acid, (c) an anti-TAT376 or anti-TAT377 polypeptide antibody, (d) a TAT376- or TAT377-binding oligopeptide, or (e) a TAT376- or TAT377-binding small organic molecule in the preparation of a medicament useful for (i) the therapeutic treatment or diagnostic detection of a cancer or tumor, or (ii) the therapeutic treatment or prevention of a cell proliferative disorder.

Another embodiment of the present invention is directed to a method for inhibiting the growth of a cancer cell, wherein the growth of said cancer cell is at least in part dependent upon the growth potentiating effect(s) of a TAT376 or TAT377 polypeptide (wherein the TAT376 or TAT377 polypeptide may be expressed either by the cancer cell itself or a cell that produces polypeptide(s) that have a growth potentiating effect on cancer cells), wherein the method comprises contacting the TAT376 or TAT377 polypeptide with an antibody, an oligopeptide or a small organic molecule that binds to the TAT376 or TAT377 polypeptide, thereby antagonizing the growth-potentiating activity of the TAT376 or TAT377 polypeptide and, in turn, inhibiting the growth of the cancer cell. Preferably the growth of the cancer cell is completely inhibited. Even more preferably, binding of the antibody, oligopeptide or small organic molecule to the TAT376 or TAT377 polypeptide induces the death of the cancer cell. Optionally, the antibody is a monoclonal antibody, antibody fragment, chimeric antibody, humanized antibody, or single-chain antibody. Antibodies, TAT376 or TAT377 binding oligopeptides and TAT376 or TAT377 binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and TAT376 or TAT377 binding oligopeptides employed in the methods of the present invention may optionally be produced in CHO cells or bacterial cells.

Yet another embodiment of the present invention is directed to a method of therapeutically treating a tumor in a mammal, wherein the growth of said tumor is at least in part dependent upon the growth potentiating effect(s) of a TAT376 or TAT377 polypeptide, wherein the method comprises administering to the mammal a therapeutically effective amount of an antibody, an oligopeptide or a small organic molecule that binds to the TAT376 or TAT377 polypeptide, thereby antagonizing the growth potentiating activity of said TAT376 or TAT377 polypeptide and resulting in the effective therapeutic treatment of the tumor. Optionally, the antibody is a monoclonal antibody, antibody fragment, chimeric antibody, humanized antibody, or single-chain antibody. Antibodies, TAT376 or TAT377 binding oligopeptides and TAT376 or TAT377 binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and oligopeptides employed in the methods of the present invention may optionally be produced in CHO cells or bacterial cells.

Yet further embodiments of the present invention will be evident to the skilled artisan upon a reading of the present specification.

### C. FURTHER ADDITIONAL EMBODIMENTS

In yet further embodiments, the invention is directed to the following set of potential claims for this application:

1. Isolated nucleic acid having a nucleotide sequence that has at least 80% nucleic acid sequence identity to:

(a) a DNA molecule encoding the amino acid sequence shown in any one of FIG. 3 or 4 (SEQ ID NOs:3 or 4);

(b) the nucleotide sequence shown in any one of FIG. 1 or 2 (SEQ ID Nos:1 or 2);

(c) the full-length coding region of the nucleotide sequence shown in any one of FIG. 1 or 2 (SEQ ID NOs:1 or 2); or

(d) the complement of (a), (b) or (c).

2. Isolated nucleic acid having:

(a) a nucleotide sequence that encodes the amino acid sequence shown in any one of FIG. 3 or 4 (SEQ ID NOs:3 or 4);

(b) the nucleotide sequence shown in any one of FIG. 1 or 2 (SEQ ID Nos:1 or 2);

(c) the full-length coding region of the nucleotide sequence shown in any one of FIG. 1 or 2 (SEQ ID Nos:1 or 2); or

(d) the complement of (a), (b) or (c).

3. Isolated nucleic acid that hybridizes to:

(a) a nucleic acid that encodes the amino acid sequence shown in any one of FIG. 3 or 4 (SEQ ID NOs:3 or 4);

(b) the nucleotide sequence shown in any one of FIG. 1 or 2 (SEQ ID Nos:1 or 2);

(c) the full-length coding region of the nucleotide sequence shown in any one of FIG. 1 or 2 (SEQ ID NOs:1 or 2); or

(d) the complement of (a), (b) or (c).

4. The nucleic acid of Claim 3, wherein the hybridization occurs under stringent conditions.

5. The nucleic acid of Claim 3 which is at least about 5 nucleotides in length.

6. An expression vector comprising the nucleic acid of Claim 1, 2 or 3.

7. The expression vector of Claim 6, wherein said nucleic acid is operably linked to control sequences recognized by a host cell transformed with the vector.

8. A host cell comprising the expression vector of Claim 7.

9. The host cell of Claim 8 which is a CHO cell, an E. coli cell or a yeast cell.

10. A process for producing a polypeptide comprising culturing the host cell of Claim 8 under conditions suitable for expression of said polypeptide and recovering said polypeptide from the cell culture.

11. An isolated polypeptide having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID Nos:1 or 2); or

(c) a polypeptide encoded by the full-length coding region of the nucleotide sequence shown in any one of FIG. 1 or 2 (SEQ ID Nos:1 or 2).

12. An isolated polypeptide having:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID NOs:1 or 2); or

(c) an amino acid sequence encoded by the full-length coding region of the nucleotide sequence shown in any one of FIG. 1 or 2 (SEQ ID Nos:1 or 2).

13. A chimeric polypeptide comprising the polypeptide of Claim 11 or 12 fused to a heterologous polypeptide.

14. The chimeric polypeptide of Claim 13, wherein said heterologous polypeptide is an epitope tag sequence or an Fc region of an immunoglobulin.

15. An isolated antibody that binds to a polypeptide having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or

(c) a polypeptide encoded by the full-length coding region of the nucleotide sequence shown in any one of FIG. 1 or 2 (SEQ ID Nos:1 or 2).

16. An isolated antibody that binds to a polypeptide having:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

(c) an amino acid sequence encoded by the full-length coding region of the nucleotide sequence shown in any one of FIG. 1 or 2 (SEQ ID Nos:1 or 2).

17. The antibody of Claim 15 or 16 which is a monoclonal antibody.

18. The antibody of Claim 15 or 16 which is an antibody fragment.

19. The antibody of Claim 15 or 16 which is a chimeric or a humanized antibody.

20. The antibody of Claim 15 or 16 which is conjugated to a growth inhibitory agent.

21. The antibody of Claim 15 or 16 which is conjugated to a cytotoxic agent.

22. The antibody of Claim 21, wherein the cytotoxic agent is selected from the group consisting of toxins, antibiotics, radioactive isotopes and nucleolytic enzymes.

23. The antibody of Claim 21, wherein the cytotoxic agent is a toxin.

24. The antibody of Claim 23, wherein the toxin is selected from the group consisting of maytansinoid and calicheamicin.

25. The antibody of Claim 23, wherein the toxin is a maytansinoid.

26. The antibody of Claim 15 or 16 which is produced in bacteria.

27. The antibody of Claim 15 or 16 which is produced in CHO cells.

28. The antibody of Claim 15 or 16 which induces death of a cell to which it binds.

29. The antibody of Claim 15 or 16 which is detectably labeled.

30. An isolated nucleic acid having a nucleotide sequence that encodes the antibody of Claim 15 or 16.

31. An expression vector comprising the nucleic acid of Claim 30 operably linked to control sequences recognized by a host cell transformed with the vector.

32. A host cell comprising the expression vector of Claim 31.

33. The host cell of Claim 32 which is a CHO cell, an E. coli cell or a yeast cell.

34. A process for producing an antibody comprising culturing the host cell of Claim 32 under conditions suitable for expression of said antibody and recovering said antibody from the cell culture.

35. An isolated oligopeptide that binds to a polypeptide having at-least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or

(c) a polypeptide encoded by the full-length coding region of the nucleotide sequence shown in any one of FIG. 1 or 2 (SEQ ID Nos:1 or 2).

36. An isolated oligopeptide that binds to a polypeptide having:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

(c) an amino acid sequence encoded by the full-length coding region of the nucleotide sequence shown in any one of FIG. 1 or 2 (SEQ ID Nos:1 or 2).

37. The oligopeptide of Claim 35 or 36 which is conjugated to a growth inhibitory agent.

38. The oligopeptide of Claim 35 or 36 which is conjugated to a cytotoxic agent.

39. The oligopeptide of Claim 38, wherein the cytotoxic agent is selected from the group consisting of toxins, antibiotics, radioactive isotopes and nucleolytic enzymes.

40. The oligopeptide of Claim 38, wherein the cytotoxic agent is a toxin.

41. The oligopeptide of Claim 40, wherein the toxin is selected from the group consisting of maytansinoid and calicheamicin.

42. The oligopeptide of Claim 40, wherein the toxin is a maytansinoid.

43. The oligopeptide of Claim 35 or 36 which induces death of a cell to which it binds.

44. The oligopeptide of Claim 35 or 36 which is detectably labeled.

45. A TAT376 or TAT377 binding organic molecule that binds to a polypeptide having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or

(c) a polypeptide encoded by the full-length coding region of the nucleotide sequence shown in any one of FIG. 1 or 2 (SEQ ID Nos:1 or 2).

46. The organic molecule of Claim 45 that binds to a polypeptide having:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

c) an amino acid sequence encoded by the full-length coding region of the nucleotide sequence shown in any one of FIG. 1 or 2 (SEQ ID Nos:1 or 2).

47. The organic molecule of Claim 45 or 46 which is conjugated to a growth inhibitory agent.

48. The organic molecule of Claim 45 or 46 which is conjugated to a cytotoxic agent.

49. The organic molecule of Claim 48, wherein the cytotoxic agent is selected from the group consisting of toxins, antibiotics, radioactive isotopes and nucleolytic enzymes.

50. The organic molecule of Claim 48, wherein the cytotoxic agent is a toxin.

51. The organic molecule of Claim 50, wherein the toxin is selected from the group consisting of maytansinoid and calicheamicin.

52. The organic molecule of Claim 50, wherein the toxin is a maytansinoid.

53. The organic molecule of Claim 45 or 46 which induces death of a cell to which it binds.

54. The organic molecule of Claim 45 or 46 which is detectably labeled.

55. A composition of matter comprising:
(a) the polypeptide of Claim 11;
(b) the polypeptide of Claim 12;
(c) the chimeric polypeptide of Claim 13;
(d) the antibody of Claim 15;
(e) the antibody of Claim 16;
(f) the oligopeptide of Claim 35;
(g) the oligopeptide of Claim 36;
(h) the TAT376 or TAT377 binding organic molecule of Claim 45; or
(i) the TAT376 or TAT377 binding organic molecule of Claim 46; in combination with a carrier.

56. The composition of matter of Claim 55, wherein said carrier is a pharmaceutically acceptable carrier.

57. An article of manufacture comprising:

(a) a container; and

(b) the composition of matter of Claim 55 contained within said container.

58. The article of manufacture of Claim 57 further comprising a label affixed to said container, or a package insert included with said container, referring to the use of said composition of matter for the therapeutic treatment of or the diagnostic detection of a cancer.

59. A method of inhibiting the growth of a cell that expresses a protein having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID Nos:1 or 2); or

(c) a polypeptide encoded by the full-length coding region of the nucleotide sequence shown in any one of FIG. 1 or 2 (SEQ ID NOs:1 or 2), said method comprising contacting said cell with an antibody, oligopeptide or organic molecule that binds to said protein, the binding of said antibody, oligopeptide or organic molecule to said protein thereby causing an inhibition of growth of said cell.

60. The method of Claim 59, wherein said antibody is a monoclonal antibody.

61. The method of Claim 59, wherein said antibody is an antibody fragment.

62. The method of Claim 59, wherein said antibody is a chimeric or a humanized antibody.

63. The method of Claim 59, wherein said antibody, oligopeptide or organic molecule is conjugated to a growth inhibitory agent.

64. The method of Claim 59, wherein said antibody, oligopeptide or organic molecule is conjugated to a cytotoxic agent.

65. The method of Claim 64, wherein said cytotoxic agent is selected from the group consisting of toxins, antibiotics, radioactive isotopes and nucleolytic enzymes.

66. The method of Claim 64, wherein the cytotoxic agent is a toxin.

67. The method of Claim 66, wherein the toxin is selected from the group consisting of maytansinoid and calicheamicin.

68. The method of Claim 66, wherein the toxin is a maytansinoid.

69. The method of Claim 59, wherein said antibody is produced in bacteria.

70. The method of Claim 59, wherein said antibody is produced in CHO cells.

71. The method of Claim 59, wherein said cell is a cancer cell.

72. The method of Claim 71, wherein said cancer cell is further exposed to radiation treatment or a chemotherapeutic agent.

73. The method of Claim 71, wherein said cancer cell is selected from the group consisting of a breast cancer cell, a colorectal cancer cell, a lung cancer cell, an ovarian cancer cell, a central nervous system cancer cell, a liver cancer cell, a bladder cancer cell, a pancreatic cancer cell, a cervical cancer cell, a melanoma cell and a leukemia cell.

74. The method of Claim 71, wherein said protein is more abundantly expressed by said cancer cell as compared to a normal cell of the same tissue origin.

75. The method of Claim 59 which causes the death of said cell.

76. The method of Claim 59, wherein said protein has:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

(c) an amino acid sequence encoded by the full-length coding region of the nucleotide sequence shown in any one of FIG. 1 or 2 (SEQ ID Nos:1 or 2).

77. A method of therapeutically treating a mammal having a cancerous tumor comprising cells that express a protein having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or

(c) a polypeptide encoded by the full-length coding region of the nucleotide sequence shown in any one of FIG. 1 or 2 (SEQ ID NOs:1 or 2), said method comprising administering to said mammal a therapeutically effective amount of an antibody, oligopeptide or organic molecule that binds to said protein, thereby effectively treating said mammal.

78. The method of Claim 77, wherein said antibody is a monoclonal antibody.

79. The method of Claim 77, wherein said antibody is an antibody fragment.

80. The method of Claim 77, wherein said antibody is a chimeric or a humanized antibody.

81. The method of Claim 77, wherein said antibody, oligopeptide or organic molecule is conjugated to a growth inhibitory agent.

82. The method of Claim 77, wherein said antibody, oligopeptide or organic molecule is conjugated to a cytotoxic agent.

83. The method of Claim 82, wherein said cytotoxic agent is selected from the group consisting of toxins, antibiotics, radioactive isotopes and nucleolytic enzymes.

84. The method of Claim 82, wherein the cytotoxic agent is a toxin.

85. The method of Claim 84, wherein the toxin is selected from the group consisting of maytansinoid and calicheamicin.

86. The method of Claim 84, wherein the toxin is a maytansinoid.

87. The method of Claim 77, wherein said antibody is produced in bacteria.

88. The method of Claim 77, wherein said antibody is produced in CHO cells.

89. The method of Claim 77, wherein said tumor is further exposed to radiation treatment or a chemotherapeutic agent.

90. The method of Claim 77, wherein said tumor is a breast tumor, a colorectal tumor, a lung tumor, an ovarian tumor, a central nervous system tumor, a liver tumor, a bladder tumor, a pancreatic tumor, or a cervical tumor.

91. The method of Claim 77, wherein said protein is more abundantly expressed by the cancerous cells of said tumor as compared to a normal cell of the same tissue origin.

92. The method of Claim 77, wherein said protein has:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID NOs:1 or 2); or

(c) an amino acid sequence encoded by the full-length coding region of the nucleotide sequence shown in any one of FIG. 1 or 2 (SEQ ID NOs:1 or 2).

93. A method of determining the presence of a protein in a sample suspected of containing said protein, wherein said protein has at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or

(c) a polypeptide encoded by the full-length coding region of the nucleotide sequence shown in any one of FIG. 1 or 2 (SEQ ID NOs:1 or 2), said method comprising exposing said sample to an antibody, oligopeptide or organic molecule that binds to said protein and determining binding of said antibody, oligopeptide or organic molecule to said protein in said sample, wherein binding of the antibody, oligopeptide or organic molecule to said protein is indicative of the presence of said protein in said sample.

94. The method of Claim 93, wherein said sample comprises a cell suspected of expressing said protein.

95. The method of Claim 94, wherein said cell is a cancer cell.

96. The method of Claim 93, wherein said antibody, oligopeptide or organic molecule is detectably labeled.

97. The method of Claim 93, wherein said protein has:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

(c) an amino acid sequence encoded by the full-length coding region of the nucleotide sequence shown in any one of FIG. 1 or 2 (SEQ ID Nos:1 or 2).

98. A method of diagnosing the presence of a tumor in a mammal, said method comprising determining the level of expression of a gene encoding a protein having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or

(c) a polypeptide encoded by the full-length coding region of the nucleotide sequence shown in any one of FIG. 1 or 2 (SEQ ID Nos:1 or 2), in a test sample of tissue cells obtained from said mammal and in a control sample of known normal cells of the same tissue origin, wherein a higher level of expression of said protein in the test sample, as compared to the control sample, is indicative of the presence of tumor in the mammal from which the test sample was obtained.

99. The method of Claim 98, wherein the step of determining the level of expression of a gene encoding said protein comprises employing an oligonucleotide in an in situ hybridization or RT-PCR analysis.

100. The method of Claim 98, wherein the step determining the level of expression of a gene encoding said protein comprises employing an antibody in an immunohistochemistry or Western blot analysis.

101. The method of Claim 98, wherein said protein has:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

(c) an amino acid sequence encoded by the full-length coding region of the nucleotide sequence shown in any one of FIG. 1 or 2 (SEQ ID Nos:1 or 2).

102. A method of diagnosing the presence of a tumor in a mammal, said method comprising contacting a test sample of tissue cells obtained from said mammal with an antibody, oligopeptide or organic molecule that binds to a protein having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or

(c) a polypeptide encoded by the full-length coding region of the nucleotide sequence shown in any one of FIG. 1 or 2 (SEQ ID Nos:1 or 2), and detecting the formation of a complex between said antibody, oligopeptide or organic molecule and said protein in the test sample, wherein the formation of a complex is indicative of the presence of a tumor in said mammal.

103. The method of Claim 102, wherein said antibody, oligopeptide or organic molecule is detectably labeled.

104. The method of Claim 102, wherein said test sample of tissue cells is obtained from an individual suspected of having a cancerous tumor.

105. The method of Claim 102, wherein said protein has:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

(c) an amino acid sequence encoded by the full-length coding region of the nucleotide sequence shown in any one of FIG. 1 or 2 (SEQ ID Nos:1 or 2).

106. A method for treating or preventing a cell proliferative disorder associated with increased expression or activity of a protein having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or

(c) a polypeptide encoded by the full-length coding region of the nucleotide sequence shown in any one of FIG. 1 or 2 (SEQ ID NOs:1 or 2), said method comprising administering to a subject in need of such treatment an effective amount of an antagonist of said protein, thereby effectively treating or preventing said cell proliferative disorder.

107. The method of Claim 106, wherein said cell proliferative disorder is cancer.

108. The method of Claim 106, wherein said antagonist is an anti-TAT376 or anti-TAT377 polypeptide antibody,TAT376 or TAT377 binding oligopeptide,TAT376 or TAT377 binding organic molecule or antisense oligonucleotide.

109. A method of binding an antibody, oligopeptide or organic molecule to a cell that expresses a protein having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID Nos:1 or 2); or

(c) a polypeptide encoded by the full-length coding region of the nucleotide sequence shown in any one of FIG. 1 or 2 (SEQ ID NOs:1 or 2), said method comprising contacting said cell with an antibody, oligopeptide or organic molecule that binds to said protein and allowing the binding of the antibody, oligopeptide or organic molecule to said protein to occur, thereby binding said antibody, oligopeptide or organic molecule to said cell.

110. The method of Claim 109, wherein said antibody is a monoclonal antibody.

111. The method of Claim 109, wherein said antibody is an antibody fragment.

112. The method of Claim 109, wherein said antibody is a chimeric or a humanized antibody.

113. The method of Claim 109, wherein said antibody, oligopeptide or organic molecule is conjugated to a growth inhibitory agent.

114. The method of Claim 109, wherein said antibody, oligopeptide or organic molecule is conjugated to a cytotoxic agent.

115. The method of Claim 114, wherein said cytotoxic agent is selected from the group consisting of toxins, antibiotics, radioactive isotopes and nucleolytic enzymes.

116. The method of Claim 114, wherein the cytotoxic agent is a toxin.

117. The method of Claim 116, wherein the toxin is selected from the group consisting of maytansinoid and calicheamicin.

118. The method of Claim 116, wherein the toxin is a maytansinoid.

119. The method of Claim 109, wherein said antibody is produced in bacteria.

120. The method of Claim 109, wherein said antibody is produced in CHO cells.

121. The method of Claim 109, wherein said cell is a cancer cell.

122. The method of Claim 121, wherein said cancer cell is further exposed to radiation treatment or a chemotherapeutic agent.

123. The method of Claim 121, wherein said cancer cell is selected from the group consisting of a breast cancer cell, a colorectal cancer cell, a lung cancer cell, an ovarian cancer cell, a central nervous system cancer cell, a liver cancer cell, a bladder cancer cell, a pancreatic cancer cell, a cervical cancer cell, a melanoma cell and a leukemia cell.

124. The method of Claim 123, wherein said protein is more abundantly expressed by said cancer cell as compared to a normal cell of the same tissue origin.

125. The method of Claim 109 which causes the death of said cell.

126. Use of a nucleic acid as claimed in any of Claims 1 to 5 or 30 in the preparation of a medicament for the therapeutic treatment or diagnostic detection of a cancer.

127. Use of a nucleic acid as claimed in any of Claims 1 to 5 or 30 in the preparation of a medicament for treating a tumor.

128. Use of a nucleic acid as claimed in any of Claims 1 to 5 or 30 in the preparation of a medicament for treatment or prevention of a cell proliferative disorder.

129. Use of an expression vector as claimed in any of Claims 6, 7 or 31 in the preparation of a medicament for the therapeutic treatment or diagnostic detection of a cancer.

130. Use of an expression vector as claimed in any of Claims 6, 7 or 31 in the preparation of medicament for treating a tumor.

131. Use of an expression vector as claimed in any of Claims 6, 7 or 31 in the preparation of a medicament for treatment or prevention of a cell proliferative disorder.

132. Use of a host cell as claimed in any of Claims 8, 9, 32, or 33 in the preparation of a medicament for the therapeutic treatment or diagnostic detection of a cancer.

133. Use of a host cell as claimed in any of Claims 8, 9, 32 or 33 in the preparation of a medicament for treating a tumor.

134. Use of a host cell as claimed in any of Claims 8, 9, 32 or 33 in the preparation of a medicament for treatment or prevention of a cell proliferative disorder.

135. Use of a polypeptide as claimed in any of Claims 11 to 14 in the preparation of a medicament for the therapeutic treatment or diagnostic detection of a cancer.

136. Use of a polypeptide as claimed in any of Claims 11 to 14 in the preparation of a medicament for treating a tumor.

137. Use of a polypeptide as claimed in any of Claims 11 to 14 in the preparation of a medicament for treatment or prevention of a cell proliferative disorder.

138. Use of an antibody as claimed in any of Claims 15 to 29 in the preparation of a medicament for the therapeutic treatment or diagnostic detection of a cancer.

139. Use of an antibody as claimed in any of Claims 15 to 29 in the preparation of a medicament for treating a tumor.

140. Use of an antibody as claimed in any of Claims 15 to 29 in the preparation of a medicament for treatment or prevention of a cell proliferative disorder.

141. Use of an oligopeptide as claimed in any of Claims 35 to 44 in the preparation of a medicament for the therapeutic treatment or diagnostic detection of a cancer.

142. Use of an oligopeptide as claimed in any of Claims 35 to 44 in the preparation of a medicament for treating a tumor.

143. Use of an oligopeptide as claimed in any of Claims 35 to 44 in the preparation of a medicament for treatment or prevention of a cell proliferative disorder.

144. Use of a TAT376 or TAT377 binding organic molecule as claimed in any of Claims 45 to 54 in the preparation of a medicament for the therapeutic treatment or diagnostic detection of a cancer.

145. Use of a TAT376 or TAT377 binding organic molecule as claimed in any of Claims 45 to 54 in the preparation of a medicament for treating a tumor.

146. Use of a TAT376 or TAT377 binding organic molecule as claimed in any of Claims 45 to 54 in the preparation of a medicament for treatment or prevention of a cell proliferative disorder.

147. Use of a composition of matter as claimed in any of Claims 55 or 56 in the preparation of a medicament for the therapeutic treatment or diagnostic detection of a cancer.

148. Use of a composition of matter as claimed in any of Claims 55 or 56 in the preparation of a medicament for treating a tumor.

149. Use of a composition of matter as claimed in any of Claims 55 or 56 in the preparation of a medicament for treatment or prevention of a cell proliferative disorder.

150. Use of an article of manufacture as claimed in any of Claims 57 or 58 in the preparation of a medicament for the therapeutic treatment or diagnostic detection of a cancer.

151. Use of an article of manufacture as claimed in any of Claims 57 or 58 in the preparation of a medicament for treating a tumor.

152. Use of an article of manufacture as claimed in any of Claims 57 or 58 in the preparation of a medicament for treatment or prevention of a cell proliferative disorder.

153. A method for inhibiting the growth of a cell, wherein the growth of said cell is at least in part dependent upon a growth potentiating effect of a protein having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID NOs:1 or 2); or (c) a polypeptide encoded by the full-length coding region of the nucleotide sequence shown in any one of FIG. 1 or 2 (SEQ ID Nos:1 or 2), said method comprising contacting said protein with an antibody, oligopeptide or organic molecule that binds to said protein, there by inhibiting the growth of said cell.

154. The method of Claim 153, wherein said cell is a cancer cell.

155. The method of Claim 153, wherein said protein is expressed by said cell.

156. The method of Claim 153, wherein the binding of said antibody, oligopeptide or organic molecule to said protein antagonizes a cell growth-potentiating activity of said protein.

157. The method of Claim 153, wherein the binding of said antibody, oligopeptide or organic molecule to said protein induces the death of said cell.

158. The method of Claim 153, wherein said antibody is a monoclonal antibody.

159. The method of Claim 153, wherein said antibody is an antibody fragment.

160. The method of Claim 153, wherein said antibody is a chimeric or a humanized antibody.

161. The method of Claim 153, wherein said antibody, oligopeptide or organic molecule is conjugated to a growth inhibitory agent.

162. The method of Claim 153, wherein said antibody, oligopeptide or organic molecule is conjugated to a cytotoxic agent.

163. The method of Claim 162, wherein said cytotoxic agent is selected from the group consisting of toxins, antibiotics, radioactive isotopes and nucleolytic enzymes.

164. The method of Claim 162, wherein the cytotoxic agent is a toxin.

165. The method of Claim 164, wherein the toxin is selected from the group consisting of maytansinoid and calicheamicin.

166. The method of Claim 164, wherein the toxin is a maytansinoid.

167. The method of Claim 153, wherein said antibody is produced in bacteria.

168. The method of Claim 153, wherein said antibody is produced in CHO cells.

169. The method of Claim 153, wherein said protein has:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

(c) an amino acid sequence encoded by the full-length coding region of the nucleotide sequence shown in any one of FIG. 1 or 2 (SEQ ID Nos:1 or 2).

170. A method of therapeutically treating a tumor in a mammal, wherein the growth of said tumor is at least in part dependent upon a growth potentiating effect of a protein having at least 80% amino acid sequence identity to:

(a) the polypeptide shown in any one of FIG. 3 or 4 (SEQ ID NOs:3 or 4);

(b) a polypeptide encoded by the nucleotide sequence shown in any one of FIG. 1 or 2 (SEQ ID Nos:1 or 2); or

(c) a polypeptide encoded by the full-length coding region of the nucleotide sequence shown in any one of FIG. 1 or 2 (SEQ ID NOs:1 or 2), said method comprising contacting said protein with an antibody, oligopeptide or organic molecule that binds to said protein, thereby effectively treating said tumor.

171. The method of Claim 170, wherein said protein is expressed by cells of said tumor.

172. The method of Claim 170, wherein the binding of said antibody, oligopeptide or organic molecule to said protein antagonizes a cell growth-potentiating activity of said protein.

173. The method of Claim 170, wherein said antibody is a monoclonal antibody.

174. The method of Claim 170, wherein said antibody is an antibody fragment.

175. The method of Claim 170, wherein said antibody is a chimeric or a humanized antibody.

176. The method of Claim 170, wherein said antibody, oligopeptide or organic molecule is conjugated to a growth inhibitory agent.

177. The method of Claim 170, wherein said antibody, oligopeptide or organic molecule is conjugated to a cytotoxic agent.

178. The method of Claim 177, wherein said cytotoxic agent is selected from the group consisting of toxins, antibiotics, radioactive isotopes and nucleolytic enzymes.

179. The method of Claim 177, wherein the cytotoxic agent is a toxin.

180. The method of Claim 179, wherein the toxin is selected from the group consisting of maytansinoid and calicheamicin.

181. The method of Claim 179, wherein the toxin is a maytansinoid.

182. The method of Claim 170, wherein said antibody is produced in bacteria.

183. The method of Claim 170, wherein said antibody is produced in CHO cells.

184. The method of Claim 170, wherein said protein has:

(a) the amino acid sequence shown in any one of FIG. 3 or 4 (SEQ ID NOs:3 or 4);

(b) an amino acid sequence encoded by the nucleotide sequence shown in any one of FIG. 1 or 2 (SEQ ID Nos:1 or 2); or

(c) an amino acid sequence encoded by the full-length coding region of the nucleotide sequence shown in any one of FIG. 1 or 2 (SEQ ID Nos:1 or 2).

### BRIEF DESCRIPTION OF THE DRAWINGS

FIG. 1 shows a nucleotide sequence (SEQ ID NO:1) of a TAT376 cDNA, wherein SEQ ID NO:1 is a clone designated herein as “DNA327307”.

FIG. 2 shows a nucleotide sequence (SEQ ID NO:2) of a TAT377 cDNA, wherein SEQ ID NO:2 is a clone designated herein as “DNA327308”.

FIG. 3 shows the amino acid sequence (SEQ ID NO:3) derived from one of the open reading frames of SEQ ID NO:1 shown in FIG. 1.

FIG. 4 shows the amino acid sequence (SEQ ID NO:4) derived from one of the open reading frames of SEQ ID NO:2 shown in FIG. 2.

FIG. 5 shows the alignment of ASCL2 mRNA (GenBank accession number AF442769) with corresponding primers, probes, amplification products and open reading frames (ORFs). Exonic material is shown as thick bars, the intervening intron is represented by a narrow bar. Positions are numbered relative to the GenBank record.

FIG. 6 shows a plot of normalized probeset intensities on chromosome 1p for a number of colon cancer cases. The 11p15 locus shows a contiguous region of probesets that are all upregulated to a similar extent in certain colonic adenocarcinomas.

FIG. 7 depicts the synthesis and labeling of in situ hybridization probes: (A) Nested PCR for probe 1061 against ASCL2 with primers 1061_P5/6; (B) Nested PCR for probe 1071 against 5′ region of ASCL2 with primers 1071_P7/8; (C, D and E) Autoradiograms of ³³P-labeled probes against the sense (S) sequence of β-actin (primers 117_P3/4), and the sense (S) and anti-sense (AS) sequences of ASCL2 (1061 and 1071) probes.

FIG. 8 shows anti-sense in situ hybridization against the 5′ region of ASCL2 (1071/HASAP) probe in a colorectal adenocarcinoma and adjacent normal mucosa. Bright-field (BF) and dark-field (DF images show no hybridization above bachground.

FIG. 9 shows the anti-sense in situ hybridization against ASCL2 (1061/HASH2) probe: (A) shows haematoxylin and eosin staining; (B) shows auto-fluorescence and phosphorimages of (C) β-actin and (D) ASCL2 (HASH2) anti-sense hybridization in a representative colorectal TMA; (E) shows auto-fluorescence and (F) phosphorimages of ASCL2 (HASH2) hybridization in a normal TMA (H2001-688). (G, H, and I) show ASCL2 (HASH2) hybridization signal, seen as silver grains, over (G) the extra-villous trophoblast cells in placental tissue and (H) the neoplastic cell population of a colorectal adenocarcinoma. There is no signal in (I) the normal colorectal mucosa. (BF, bright field; DF, dark field)

FIG. 10 shows the primer-probe set validation for quantitative RT-PCR, using Hs.Scute_f/r/p1 as an example: (A) the size and presence of RT-PCR products was checked on a 4% agarose gel. The RPL19 reference gene (amplification product=68 bp) was highly expressed in normal and malignant tissue. ASCL2 (amplification product=62 bp) was more abundant in malignant tissue; (B) shows the semi-log amplification plots of RPL19 and ASCL2 primer-probe sets across eight two-fold serial dilutions of genomic DNA (200 ng to 3.125 ng). With each two-fold dilution the cycle threshold (Ct) dropped by one; (C) the relative efficiency of the reference and experimental primer-probe sets was assessed by plotting the Δ(RPL19-ASCL2) Ct against the log input RNA amount. The gradient was equal to 0.07.

FIGS. 11A-11B: FIG. 11A shows ASCL2 (Hs.Scute_f/r/p1) fold-change in colorectal tissues and cell lines, quantified by real-time RT-PCR. Samples were normalized to the reference gene RPL19 and the expression in normal colorectal mucosa, when available. Otherwise, cases and cell lines (marked with an asterix) were normalized to the mean ΔCt of all normal colorectal samples; FIG. 11B shows the ΔΔCt values comparing the amplification of three primer-probe sets designed against different regions of ASCL2, Data is shown for high (HCT15), mid (COLO205, JEG3) and low-expressing (HCT116) cell lines and was normalized to RPL19.

FIG. 12 depicts ASCL2 mRNA corresponding to the known full-length gene sequence identified as AF442769 in GenBank. The full-length mRNA unspliced transcript contains two exons with two open reading frames [shown as HASAP ORF and HASH2 ORF respectively], the first open reading frame within the first exon is identified as encoding a polypeptide designated as HASAP; the second open reading frame within the first exon is identified as encoding a polypeptide designated as HASH2. The spliced mRNA transcript corresponds to a splice within the first exon of the full-length mRNA transcript which encodes the polypeptide designated as HASH2.

FIG. 13 shows PCR-based cloning of the HASH2 open reading frame: (A-D) show agarose gels (1.2%) stained with etidium bromide; (A) shows amplification of the open reading frame template from a colorectal adenocarcinoma cDNA library (BD Clontech) and HCT15 cDNA, rounds 1 (R1) and 2 (R2); (B) shows amplification of open reading frame template with 327308.XhoI/HindIII primers; (C) shows HindIII and XhoI restriction of the open reading frame to create sticky ends, prior to gel-purification; (D) shows restriction of the ligated vector with Pst to confirm the presence of the desired insert; (E) shows the vector map of pEGFP-N1 vector with a CMV promoter, Kan^{r} gene and EGFP tag. The insert is ligated into the MCS.

FIG. 14 shows PCR-based cloning of the HASAP open reading frame: (A-D) show agarose gels (1.2%) stained with ethidium bromide; (A) shows nested amplification of the open reading frame template from a placental cDNA library (BD Clontech) and HCT15 cDNA, rounds 1 (R1) and 2 (R2); (B) shows amplification of open reading frame template with 327307.XhoII/HindIII primers; (C) shows HindIII and XhoI restriction of the open reading frame to create sticky ends, prior to gel-purification; (D) shows restriction of the ligated vector with SmaI to confirm the presence of the insert.

FIG. 15 shows autoradiograms of Northern blots directed against β-actin and ASCL2 (HASH2-N-F/R1): (A and B) show blots of cell-line RNA from HCT15, DLD-1, JEG3 and HCT116; (A) the β-actin probe hybridized to all lanes to give a single band of approximately equal intensity in each lane; (B) the ASCL2 (HASH2) probe hybridized to give a single band (1470 bp) and showed the strongest signal against HCT15, with reduced intensity in DLD-1 and no appreciable signal in JEG3 or HCT116; (c) shows Commercial blot of normal tissue RNA, hybridized with the ASCL2 (HASH2) probe. Single bands at 1470 bp were evident in RNA from the placenta and small intestine only. Key: 1, peripheral blood leukocytes; 2, lung; 3, placenta; 4, small intestine; 5, liver; 6, kidney; 7, spleen; 8, thymus; 9, colon; 10, skeletal muscle; 11, heart; 12, brain. Ribosomal RNA 28s and 18s bands have molecular weight of 4718 and 1874 bp respectively.

FIG. 16 shows the results of library screening for the HASAP open reading frame: (A and B) shows initial PCR-based screening of in-house cDNA libraries and HCT15 cDNA, rounds 1 (R1) and 2 (R2). The open-reading frame was amplified from HCT15 cDNA and LIB687, which was used for further analysis. Key: LIB380, normal placenta >2.0 kpb; LIB381, normal placenta 0.6-2.0 kbp; LIB687, COLO205 >2.0 kpb; LIB688, COLO205 0.6-2.0 kpb; LIB835, normal colon >2.0 kpb; LIB836, normal colon 0.6-2.0 kpb; (C) shows an autogram of a nitrocellulose filter from a colony lift, hybridized with HASAP-N-F/R3 probe; (D) shows restriction of four cloned vectors with XbaI to confirm the presence of the desired insert.

FIG. 17 shows plots of cytometry cell counts by fluorescent intensity for HCT15 cells FITC-labeled with antibodies against HASAP, HASH2 and c-Myc. The percentage of positive cells above the threshold is noted on each plot. Pre-immune sera and rabbit immunoglobulins were included as negative controls.

FIG. 18 shows Western blots of denatured nuclear protein lysates from colorectal cancer cell lines: (A) shows Coomassie blue stain of protein lysates with molecular weight markers (Seeblue+2 and Mark 12) showing integrity of the protein and equal loading; (B, C, D, and E) show Hyperfilms exposed to Western blots, probed with antibodies against c-Myc, HASAP and HASH2.

FIG. 19 shows the comparison of human ASCL2 mRNA with the genomic region 5′ to murine MASH2. FIGS. 19A and 19B show the positions of start/stop codons and hydropathy plots are shown for the three open reading frames. The HASAP open reading frame has 38.2% synteny with the corresponding region in the mouse.

### DETAILED DESCRIPTION OF THE PREFERRED EMBODIMENTS

I. Definitions

The terms “TAT polypeptide” and “TAT” as used herein and when immediately followed by a numerical designation, refer to various polypeptides, wherein the complete designation (i.e., TAT/number) refers to specific polypeptide sequences as described herein. The terms “TAT/number polypeptide” and “TAT/number” wherein the term “number” is provided as an actual numerical designation as used herein encompass native sequence polypeptides, polypeptide variants and fragments of native sequence polypeptides and polypeptide variants (which are further defined herein). The TAT polypeptides described herein may be isolated from a variety of sources, such as from human tissue types or from another source, or prepared by recombinant or synthetic methods. The term “TAT polypeptide” refers to each individual TAT/number polypeptide disclosed herein. All disclosures in this specification which refer to the “TAT polypeptide” refer to each of the polypeptides individually as well as jointly. For example, descriptions of the preparation of, purification of, derivation of, formation of antibodies to or against, formation of TAT binding oligopeptides to or against, formation of TAT binding organic molecules to or against, administration of, compositions containing, treatment of a disease with, etc., pertain to each polypeptide of the invention individually. The term “TATpolypeptide” also includes variants of the TAT/number polypeptides disclosed herein.

The term “Achaete-Scute Like 2” or “ASCL2” corresponds to known full-length gene sequence identified as AF442769 in GenBank. The full-length unspliced transcript contains two exons with two open reading frames, the first open reading frame within the first exon is identified as encoding a polypeptide designated as “TAT376” (also synonymous with the name HASAP); the second open reading frame within the first exon is identified as encoding a polypeptide designated as “TAT377” (also synonymous with the name HASH2). Thus, reference to a “TAT376” polypeptide is meant to be interchangeable with a HASAP polypeptide and vice versa. Reference to a “TAT377” polypepetide is meant to be interchangeable with a HASH2 polypeptide and vice versa.

The term “spliced ASCL2” transcript corresponds to a splice within the first exon of the unspliced full-length gene which encodes the transcript identified herein as “TAT377” (also synonymous with the name HASH2). Reference to a “TAT377” polypepetide is meant to be interchangeable with a HASH2 polypeptide and vice versa.

A “native sequence TAT376 or TAT377 polypeptide” comprises a polypeptide having the same amino acid sequence as the corresponding TAT376 or TAT377 polypeptide derived from nature. Such native sequence TAT376 or TAT377 polypeptides can be isolated from nature or can be produced by recombinant or synthetic means. The term “native sequence TAT376 or TAT377 polypeptide” specifically encompasses naturally-occurring truncated or secreted forms of the specific TAT376 or TAT377 polypeptide (e.g., an extracellular domain sequence), naturally-occurring variant forms (e.g., alternatively spliced forms) and naturally-occurring allelic variants of the polypeptide. In certain embodiments of the invention, the native sequence TAT376 or TAT377 polypeptides disclosed herein are mature or full-length native sequence polypeptides comprising the full-length amino acids sequences shown in the accompanying figures. Start and stop codons (if indicated) are shown in bold font and underlined in the figures. Nucleic acid residues indicated as “N” in the accompanying figures are any nucleic acid residue. However, while the TAT376 or TAT377 polypeptides disclosed in the accompanying figures are shown to begin with methionine residues designated herein as amino acid position I in the figures, it is conceivable and possible that other methionine residues located either upstream or downstream from the amino acid position 1 in the figures may be employed as the starting amino acid residue for the TAT376 or TAT377 polypeptides.

The approximate location of the “signal peptides” of the various TAT376 or TAT377 polypeptides disclosed herein may be shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10: 1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention. “TAT376 or TAT377 polypeptide variant” means a TAT376 or TAT377 polypeptide, preferably an active TAT376 or TAT377 polypeptide, as defined herein having at least about 80% amino acid sequence identity with a full-length native sequence TAT376 or TAT377 polypeptide sequence as disclosed herein, a TAT376 or TAT377 polypeptide sequence lacking the signal peptide as disclosed herein, an extracellular domain of a TAT376 or TAT377 polypeptide, with or without the signal peptide, as disclosed herein or any other fragment of a full-length TAT376 or TAT377 polypeptide sequence as disclosed herein (such as those encoded by a nucleic acid that represents only a portion of the complete coding sequence for a full-length TAT376 or TAT377 polypeptide). Such TAT376 or TAT377 polypeptide variants include, for instance, TAT376 or TAT377 polypeptides wherein one or more amino acid residues are added, or deleted, at the N- or C-terminus of the full-length native amino acid sequence. Ordinarily, a TAT376 or TAT377 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence TAT376 or TAT377 polypeptide sequence as disclosed herein, a TAT376 or TAT377 polypeptide sequence lacking the signal peptide as disclosed herein, an extracellular domain of a TAT376 or TAT377 polypeptide, with or without the signal peptide, as disclosed herein or any other specifically defined fragment of a full-length TAT376 or TAT377 polypeptide sequence as disclosed herein. Ordinarily, TAT variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, TAT variant polypeptides will have no more than one conservative amino acid substitution as compared to the native TAT376 or TAT377 polypeptide sequence, alternatively no more than 2, 3, 4, 5, 6, 7, 8, 9, or 10 conservative amino acid substitution as compared to the native TAT376 or TAT377 polypeptide sequence.

“Percent (%) amino acid sequence identity” with respect to the TAT376 or TAT377 polypeptide sequences identified herein is defined as the percentage of amino acid residues in a candidate sequence that are identical with the amino acid residues in the specific TAT376 or TAT377 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “TAT”, wherein “TAT” represents the amino acid sequence of a hypothetical TAT376 or TAT377 polypeptide of interest, “Comparison Protein” represents the amino acid sequence of a polypeptide against which the “TAT” polypeptide of interest is being compared, and “X”, “Y” and “Z” each represent different hypothetical amino acid residues. Unless specifically stated otherwise, all % amino acid sequence identity values used herein are obtained as described in the immediately preceding paragraph using the ALIGN-2 computer program.

“TAT variant polynucleotide” or “TAT variant nucleic acid sequence” means a nucleic acid molecule which encodes a TAT376 or TAT377 polypeptide, preferably an active TAT376 or TAT377 polypeptide, as defined herein and which has at least about 80% nucleic acid sequence identity with a nucleotide acid sequence encoding a full-length native sequence TAT376 or TAT377 polypeptide sequence as disclosed herein, a full-length native sequence TAT376 or TAT377 polypeptide sequence lacking the signal peptide as disclosed herein, an extracellular domain of a TAT376 or TAT377 polypeptide, with or without the signal peptide, as disclosed herein or any other fragment of a full-length TAT376 or TAT377 polypeptide sequence as disclosed herein (such as those encoded by a nucleic acid that represents only a portion of the complete coding sequence for a full-length TAT376 or TAT377 polypeptide). Ordinarily, a TAT variant polynucleotide will have at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding a full-length native sequence TAT376 or TAT377 polypeptide sequence as disclosed herein, a full-length native sequence TAT376 or TAT377 polypeptide sequence lacking the signal peptide as disclosed herein, an extracellular domain of a TAT376 or TAT377 polypeptide, with or without the signal sequence, as disclosed herein or any other fragment of a full-length TAT376 or TAT377 polypeptide sequence as disclosed herein. Variants do not encompass the native nucleotide sequence.

Ordinarily, TAT variant polynucleotides are at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Percent (%) nucleic acid sequence identity” with respect to TAT376- or TAT377-encoding nucleic acid sequences identified herein is defined as the percentage of nucleotides in a candidate sequence that are identical with the nucleotides in the TAT nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal.the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “TAT-DNA”, wherein “TAT-DNA” represents a hypothetical TAT-encoding nucleic acid sequence of interest, “Comparison DNA” represents the nucleotide sequence of a nucleic acid molecule against which the “TAT-DNA” nucleic acid molecule of interest is being compared, and “N”, “L” and “V” each represent different hypothetical nucleotides. Unless specifically stated otherwise, all % nucleic acid sequence identity values used herein are obtained as described in the immediately preceding paragraph using the ALIGN-2 computer program.

In other embodiments, TAT376 or TAT377 variant polynucleotides are nucleic acid molecules that encode a TAT376 or TAT377 polypeptide and which are capable of hybridizing, preferably under stringent hybridization and wash conditions, to nucleotide sequences encoding a full-length TAT376 or TAT377 polypeptide as disclosed herein. TAT376 or TAT377 variant polypeptides may be those that are encoded by a TAT376 or TAT377 variant polynucleotide.

The term “full-length coding region” when used in reference to a nucleic acid encoding a TAT376 or TAT377 polypeptide refers to the sequence of nucleotides which encode the full-length TAT376 or TAT377 polypeptide of the invention (which is often shown between start and stop codons, inclusive thereof, in the accompanying figures). The term “full-length coding region” when used in reference to an ATCC deposited nucleic acid refers to the TAT376 or TAT377 polypeptide-encoding portion of the cDNA that is inserted into the vector deposited with the ATCC (which is often shown between start and stop codons, inclusive thereof, in the accompanying figures).

“Isolated,” when used to describe the various TAT376 or TAT377 polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the TAT376 or TAT377 polypeptide natural environment will not be present. Ordinarily, however, isolated polypeptide will be prepared by at least one purification step.

An “isolated” TAT376 or TAT377 polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and %SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a TAT376 or TAT377 polypeptide or anti-TAT376 or anti-TAT377 antibody fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of a TAT376 or TAT377 polypeptide which retain a biological and/or an immunological activity of native or naturally-occurring TAT, wherein “biological” activity refers to a biological function (either inhibitory or stimulatory) caused by a native or naturally-occurring TAT376 or TAT377 other than the ability to induce the production of an antibody against an antigenic epitope possessed by a native or naturally-occurring TAT376 or TAT377 and an “immunological” activity refers to the ability to induce the production of an antibody against an antigenic epitope possessed by a native or naturally-occurring TAT376 or TAT377.

The term “antagonist” is used in the broadest sense, and includes any molecule that partially or fully blocks, inhibits, or neutralizes a biological activity of a native TAT376 or TAT377 polypeptide disclosed herein. In a similar manner, the term “agonist” is used in the broadest sense and includes any molecule that mimics a biological activity of a native TAT376 or TAT377 polypeptide disclosed herein. Suitable agonist or antagonist molecules specifically include agonist or antagonist antibodies or antibody fragments, fragments or amino acid sequence variants of native TAT376 or TAT377 polypeptides, peptides, antisense oligonucleotides, small organic molecules, etc. Methods for identifying agonists or antagonists of a TAT376 or TAT377 polypeptide may comprise contacting a TAT376 or TAT377 polypeptide with a candidate agonist or antagonist molecule and measuring a detectable change in one or more biological activities normally associated with the TAT376 or TAT377 polypeptide.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a TAT376 or TAT377 polypeptide-expressing cancer if, after receiving a therapeutic amount of an anti-TAT376 or anti-TAT377 antibody, TAT376 or TAT377 binding oligopeptide or TAT376 or TAT377 binding organic molecule according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the anti-TAT376 or anti-TAT377 antibody or TAT376 or TAT377 binding oligopeptide may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

For bladder cancer, which is a more localized cancer, methods to determine progress of disease include urinary cytologic evaluation by cystoscopy, monitoring for presence of blood in the urine, visualization of the urothelial tract by sonography or an intravenous pyelogram, computed tomography (CT) and magnetic resonance imaging (MRI). The presence of distant metastases can be assessed by CT of the abdomen, chest x-rays, or radionuclide imaging of the skeleton.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of the treatment of, alleviating the symptoms of or diagnosis of a cancer refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

By “solid phase” or “solid support” is meant a non-aqueous matrix to which an antibody, TAT376 or TAT377 binding oligopeptide or TAT376 or TAT377 binding organic molecule of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a TAT376 or TAT377 polypeptide, an antibody thereto or a TAT376 or TAT377 binding oligopeptide) to a mammal. The components of the liposome are commonly arranged in a bilayer formation, similar to the lipid arrangement of biological membranes.

A “small” molecule or “small” organic molecule is defined herein to have a molecular weight below about 500 Daltons.

An “effective amount” of a polypeptide, antibody, TAT376 or TAT377 binding oligopeptide, TAT376 or TAT377 binding organic molecule or an agonist or antagonist thereof as disclosed herein is an amount sufficient to carry out a specifically stated purpose. An “effective amount” may be determined empirically and in a routine manner, in relation to the stated purpose.

The term “therapeutically effective amount” refers to an amount of an antibody, polypeptide, TAT376 or TAT377 binding oligopeptide, TAT376 or TAT377 binding organic molecule or other drug effective to “treat” a disease or disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

A “growth inhibitory amount” of an anti-TAT376 or anti-TAT377 antibody, TAT376 or TAT377 polypeptide, TAT376 or TAT377 binding oligopeptide or TAT376 or TAT377 binding organic molecule is an amount capable of inhibiting the growth of a cell, especially tumor, e.g., cancer cell, either in vitro or in vivo.

A “growth inhibitory amount” of an anti-TAT376 or anti-TAT377 antibody, TAT376 or TAT377 polypeptide, TAT376 or TAT377 binding oligopeptide or TAT376 or TAT377 binding organic molecule for purposes of inhibiting neoplastic cell growth may be determined empirically and in a routine manner.

A “cytotoxic amount” of an anti-TAT376 or anti-TAT377 antibody, TAT376 or TAT377 polypeptide, TAT376 or TAT377 binding oligopeptide or TAT376 or TAT377 binding organic molecule is an amount capable of causing the destruction of a cell, especially tumor, e.g., cancer cell, either in vitro or in vivo. A “cytotoxic amount” of an anti-TAT376 or anti-TAT377 antibody, TAT376 or TAT377 polypeptide, TAT376 or TAT377 binding oligopeptide or TAT376 or TAT377 binding organic molecule for purposes of inhibiting neoplastic cell growth may be determined empirically and in a routine manner.

The term “antibody” is used in the broadest sense and specifically covers, for example, single anti-TAT376 or anti-TAT377 monoclonal antibodies (including agonist, antagonist, and neutralizing antibodies), anti-TAT376 or anti-TAT377 antibody compositions with polyepitopic specificity, polyclonal antibodies, single chain anti-TAT376 or anti-TAT377 antibodies, and fragments of anti-TAT376 or anti-TAT377 antibodies (see below) as long as they exhibit the desired biological or immunological activity. The term “immunoglobulin” (Ig) is used interchangeable with antibody herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 1-35 (H1), 50-65 (H2) and 95-102 (H3) in the V_{H}; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 53-55 (H2) and 96-101 (H3) in the V_{H}; Chothia and Lesk J. Mol. Biol. 196:901-917 (1987)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a C_{L} and at least heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

A “TAT376 or TAT377 binding oligopeptide” is an oligopeptide that binds, preferably specifically, to a TAT376 or TAT377 polypeptide as described herein. TAT376 or TAT377 binding oligopeptides may be chemically synthesized using known oligopeptide synthesis methodology or may be prepared and purified using recombinant technology. TAT376 or TAT377 binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a TAT376 or TAT377 polypeptide as described herein. TAT376 or TAT377 binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:4363, and Smith, G. P. (1991) Current Opin. Biotechnol., 2:668).

A “TAT376 or TAT377 binding organic molecule” is an organic molecule other than an oligopeptide or antibody as defined herein that binds, preferably specifically, to a TAT376 or TAT377 polypeptide as described herein. TAT376 or TAT377 binding organic molecules may be identified and chemically synthesized using known methodology (see, e.g., PCT Publication Nos. WO00/00823 and WO00/39585). TAT376 or TAT377 binding organic molecules are usually less than about 2000 daltons in size, alternatively less than about 1500, 750, 500, 250 or 200 daltons in size, wherein such organic molecules that are capable of binding, preferably specifically, to a TAT376 or TAT377 polypeptide as described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585).

An antibody, oligopeptide or other organic molecule “which binds” an antigen of interest, e.g. a tumor-associated polypeptide antigen target, is one that binds the antigen with sufficient affinity such that the antibody, oligopeptide or other organic molecule is useful as a diagnostic and/or therapeutic agent in targeting a cell or tissue expressing the antigen, and does not significantly cross-react with other proteins. In such embodiments, the extent of binding of the antibody, oligopeptide or other organic molecule to a “non-target” protein will be less than about 10% of the binding of the antibody, oligopeptide or other organic molecule to its particular target protein as determined by fluorescence activated cell sorting (FACS) analysis or radioimmunoprecipitation (RIA). With regard to the binding of an antibody, oligopeptide or other organic molecule to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope.

An antibody, oligopeptide or other organic molecule that “inhibits the growth of tumor cells expressing a TAT376 or TAT377 polypeptide” or a “growth inhibitory” antibody, oligopeptide or other organic molecule is one which results in measurable growth inhibition of cancer cells expressing or overexpressing the appropriate TAT376 or TAT377 polypeptide. The TAT376 or TAT377 polypeptide may be a transmembrane polypeptide expressed on the surface of a cancer cell or may be a polypeptide that is produced and secreted by a cancer cell. Preferred growth inhibitory anti-TAT376 or anti-TAT377 antibodies, oligopeptides or organic molecules inhibit growth of TAT376- or TAT377-expressing tumor cells by greater than 20%, preferably from about 20% to about 50%, and even more preferably, by greater than 50% (e.g., from about 50% to about 100%) as compared to the appropriate control, the control typically being tumor cells not treated with the antibody, oligopeptide or other organic molecule being tested. In one embodiment, growth inhibition can be measured at an antibody concentration of about 0.1 to 30 μg/ml or about 0.5 nM to 200 nM in cell culture, where the growth inhibition is determined 1-10 days after exposure of the tumor cells to the antibody. Growth inhibition of tumor cells in vivo can be determined in various ways such as is described in the Experimental Examples section below. The antibody is growth inhibitory in vivo if administration of the anti-TAT376 or anti-TAT377 antibody at about 1 μg/kg to about 100 mg/kg body weight results in reduction in tumor size or tumor cell proliferation within about 5 days to 3 months from the first administration of the antibody, preferably within about 5 to 30 days.

An antibody, oligopeptide or other organic molecule which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one which overexpresses a TAT376 or TAT377 polypeptide. Preferably the cell is a tumor cell, e.g., a prostate, breast, ovarian, stomach, endometrial, lung, kidney, colon, bladder cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody, oligopeptide or other organic molecule which induces apoptosis is one which results in about 2 to 50 fold, preferably about 5 to 50 fold, and most preferably about 10 to 50 fold, induction of annexin binding relative to untreated cell in an annexin binding assay.

Antibody “effector functions” refer to those biological activities attributable to the Fc region (a native sequence Fc region or amino acid sequence variant Fc region) of an antibody, and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. (USA) 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:2429 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

An antibody, oligopeptide or other organic molecule which “induces cell death” is one which causes a viable cell to become nonviable. The cell is one which expresses a TAT376 or TAT377 polypeptide, preferably a cell that overexpresses a TAT376 or TAT377 polypeptide as compared to a normal cell of the same tissue type. The TAT376 or TAT377 polypeptide may be a transmembrane polypeptide expressed on the surface of a cancer cell or may be a polypeptide that is produced and secreted by a cancer cell. Preferably, the cell is a cancer cell, e.g., a breast, ovarian, stomach, endometrial, salivary gland, lung, kidney, colon, thyroid, pancreatic or bladder cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the antibody, oligopeptide or other organic molecule is able to induce cell death, loss of membrane integrity as evaluated by uptake of propidium iodide (PI), trypan blue (see Moore et al. Cytotechnology 17:1-11 (1995)) or 7AAD can be assessed relative to untreated cells. Preferred cell death-inducing antibodies, oligopeptides or other organic molecules are those which induce PI uptake in the PI uptake assay in BT474 cells.

A “TAT376- or TAT377-expressing cell” is a cell which expresses an endogenous or transfected TAT376 or TAT377 polypeptide either on the cell surface or in a secreted form. A “TAT376- or TAT377-expressing cancer” is a cancer comprising cells that have a TAT376 or TAT377 polypeptide present on the cell surface or that produce and secrete a TAT376 or TAT377 polypeptide. A “TAT376- or TAT377-expressing cancer” optionally produces sufficient levels of TAT376 or TAT377 polypeptide on the surface of cells thereof, such that an anti-TAT376 or anti-TAT377 antibody, oligopeptide ot other organic molecule can bind thereto and have a therapeutic effect with respect to the cancer. In another embodiment, a “TAT376- or TAT377-expressing cancer” optionally produces and secretes sufficient levels of TAT376 or TAT377 polypeptide, such that an anti-TAT376 or anti-TAT377 antibody, oligopeptide ot other organic molecule antagonist can bind thereto and have a therapeutic effect with respect to the cancer. With regard to the latter, the antagonist may be an antisense oligonucleotide which reduces, inhibits or prevents production and secretion of the secreted TAT376 or TAT377 polypeptide by tumor cells. A cancer which “overexpresses” a TAT376 or TAT377 polypeptide is one which has significantly higher levels of TAT376 or TAT377 polypeptide at the cell surface thereof, or produces and secretes, compared to a noncancerous cell of the same tissue type. Such overexpression may be caused by gene amplification or by increased transcription or translation. TAT376 or TAT377 polypeptide overexpression may be determined in a diagnostic or prognostic assay by evaluating increased levels of the TAT376 or TAT377 protein present on the surface of a cell, or secreted by the cell (e.g., via an immunohistochemistry assay using anti-TAT376 or anti-TAT377 antibodies prepared against an isolated TAT376 or TAT377 polypeptide which may be prepared using recombinant DNA technology from an isolated nucleic acid encoding the TAT376 or TAT377 polypeptide; FACS analysis, etc.). Alternatively, or additionally, one may measure levels of TAT376 or TAT377 polypeptide-encoding nucleic acid or mRNA in the cell, e.g., via fluorescent in situ hybridization using a nucleic acid based probe corresponding to a TAT376- or TAT377-encoding nucleic acid or the complement thereof; (FISH; see WO98/45479 published October, 1998), Southern blotting, Northern blotting, or polymerase chain reaction (PCR) techniques, such as real time quantitative PCR (RT-PCR). One may also study TAT376 or TAT377 polypeptide overexpression by measuring shed antigen in a biological fluid such as serum, e.g, using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al., J. Immunol. Methods 132:73-80 (1990)). Aside from the above assays, various in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the antibody.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The word “label” when used herein refers to a detectable compound or composition which is conjugated directly or indirectly to the antibody, oligopeptide or other organic molecule so as to generate a “labeled” antibody, oligopeptide or other organic molecule. The label may be detectable by itself (e.g. radioisotope labels or fluorescent labels) or, in the case of an enzymatic label, may catalyze chemical alteration of a substrate compound or composition which is detectable.

“Replication-preventing agent” is an agent wherein replication, function, and/or growth of the cells is inhibited or prevented, or cells are destroyed, no matter what the mechanism, such as by apoptosis, angiostasis, cytosis, tumoricide, mytosis inhibition, blocking cell cycle progression, arresting cell growth, binding to tumors, acting as cellular mediators, etc. Such agents include a chemotherapeutic agent, cytotoxic agent, cytokine, growth-inhibitory agent, or anti-hormonal agent, e.g., an anti-estrogen compound such as tamoxifen, an anti-progesterone such as onapristone (see, EP 616 812); or an anti-androgen such as flutamide, as well as aromidase inhibitors, or a hormonal agent such as an androgen.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

Preferred cytotoxic agents herein for the specific tumor types to use in combination with the antagonists herein are as follows:
1. Prostate cancer: androgens, docetaxel, paclitaxel, estramustine, doxorubicin, mitoxantrone, antibodies to ErbB2 domain(s) such as 2C4 (WO 01/00245; hybridoma ATCC HB-12697), which binds to a region in the extracellular domain of ErbB2 (e.g., any one or more residues in the region from about residue 22 to about residue 584 of ErbB2, inclusive), AVASTINTM anti-vascular endothelial growth factor (VEGF), TARCEVATM OSI-774 (erlotinib) (Genenetech and OSI Pharmaceuticals), or other epidermal growth factor receptor tyrosine kinase inhibitors (EGFR TKI's).
2. Stomach cancer: 5-fluorouracil (5FU), XELODATM capecitabine, methotrexate, etoposide, cisplatin/carboplatin, pacliitaxel, docetaxel, gemcitabine, doxorubicin, and CPT-11 (camptothcin-11; irinotecan, USA Brand Name: CAMPTOSAR®).
3. Pancreatic cancer: gemcitabine, 5FU, XELODATM capecitabine, CPT-11, docetaxel, paclitaxel, cisplatin, carboplatin, TARCEVATM erlotinib, and other EGFR TKI's.
4. Colorectal cancer: 5FU, XELODATM capecitabine, CPT-11, oxaliplatin, AVASTINTM anti-VEGF, TARCEVATM erlotinib and other EGFR TKI's, and ERBITUX™ (formerly known as IMC-C225) human:murine-chimerized monoclonal antibody that binds to EGFR and blocks the ability of EGF to initiate receptor activation and signaling to the tumor.
5. Renal cancer: IL-2, interferon alpha, AVASTINTM anti-VEGF, MEGACETM (Megestrol acetate) progestin, vinblastine, TARCEVATM erlotinib, and other EGFR TKI's.

A “growth inhibitory agent” when used herein refers to a compound or composition which inhibits growth of a cell, especially a TAT376- or TAT377-expressing cancer cell, either in vitro or in vivo. Thus, the growth inhibitory agent may be one which significantly reduces the percentage of TAT376- or TAT377-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

TABLE 2

| | | |
|---|---|---|
| TAT | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the TAT376 or TAT377 polypeptide) = 5 divided by 15 = 33.3%

TABLE 3

| | |
|---|---|
| TAT | XXXXXXXXXX |
| | (Length = 10 amino acids) |
| Comparison Protein | XXXXXYYYYYYZZYZ |
| | (Length = 15 amino acids) |

% amino acid sequence identity = (the number of identically matching amino acid residues between the two polypeptide sequences as determined by ALIGN-2) divided by (the total number of amino acid residues of the TAT376 or TAT377 polypeptide) = 5 divided by 10 = 50%

TABLE 4

| | | |
|---|---|---|
| TAT-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the TAT-DNA nucleic acid sequence) = 6 divided by 14 = 42.9%

TABLE 5

| | | |
|---|---|---|
| TAT-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the TAT-DNA nucleic acid sequence) = 4 divided by 12 = 33.3%

II. Compositions and Methods of the Invention

A. Anti-TAT376 or Anti-TAT377 Antibodies

In one embodiment, the present invention provides anti-TAT376 or anti-TAT377 antibodies which may find use herein as therapeutic and/or diagnostic agents. Exemplary antibodies include polyclonal, monoclonal, humanized, bispecific, and heteroconjugate antibodies.

1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

3. Human and Humanized Antibodies

The anti-TAT376 or anti-TAT377 antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-TAT376 or anti-TAT377 antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); U.S. Pat. No. 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

4. Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

5. Bispecific Antibodies

Bispecific antibodies are antibodies that have binding specificities for at least two different epitopes. Exemplary bispecific antibodies may bind to two different epitopes of a TAT376 or TAT377 protein as described herein. Other such antibodies may combine a TAT376 or TAT377 binding site with a binding site for another protein. Alternatively, an anti-TAT376 or anti-TAT377 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the TAT376- or TAT377-expressing cell. Bispecific antibodies may also be used to localize cytotoxic agents to cells which express TAT376 or TAT377. These antibodies possess a TAT376- or TAT377-binding arm and an arm which binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming-two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147:60 (1991).

6. Heteroconiugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

7. Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

8. Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

9. Immunoconjugate

The invention also pertains to immunoconjugates comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothene, and CC1065, and the derivatives of these toxins that have toxin activity, are also contemplated herein.

Maytansine and Maytansinoids

In one preferred embodiment, an anti-TAT antibody (full length or fragments) of the invention is conjugated to one or more maytansinoid molecules.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansinoid-Antibody Conjugates

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansonid drug, which could be increased by increasing the number of maytansinoid molecules per antibody molecule. The A7-maytansinoid conjugate showed low systemic cytotoxicity in mice.

Anti-TAT376 or Anti-TAT377 Polypeptide Antibody-Maytansinoid Conjugates (Immunoconjugates)

Anti-TAT376 or anti-TAT377 antibody-maytansinoid conjugates are prepared by chemically linking an anti-TAT376 or anti-TAT377 antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disufide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Calicheamicin

Another immunoconjugate of interest comprises an anti-TAT376 or anti-TAT377 antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ₁^{I} (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other Cytotoxic Agents

Other antitumor agents that can be conjugated to the anti-TAT376 or anti-TAT377 antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleuritesfordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated anti-TAT376 or anti-TAT377 antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

Alternatively, a fusion protein comprising the anti-TAT376 or anti-TAT377 antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

10. Immunoliposomes

The anti-TAT376 or anti-TAT377 antibodies disclosed herein may also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

B. TAT376 or TAT377 Binding Oligopeptides

TAT376 or TAT377 binding oligopeptides of the present invention are oligopeptides that bind, preferably specifically, to a TAT376 or TAT377 polypeptide as described herein. TAT376 or TAT377 binding oligopeptides may be chemically synthesized using known oligopeptide synthesis methodology or may be prepared and purified using recombinant technology. TAT376 or TAT377 binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a TAT376 or TAT377 polypeptide as described herein. TAT376 or TAT377 binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988) Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify.member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren, Z-J. et al. (1998) Gene 215:439; Zhu, Z (1997) CAN 33:534; Jiang, J. et al. (1997) can 128:44380; Ren, Z-J. et al. (1997) CAN 127:215644; Ren, Z-J. (1996) Protein Sci. 5:1833; Efimov, V. P. et al. (1995) Virus Genes 10:173) and T7 phage display systems (Smith, G. P. and Scott, J. K. (1993) Methods in Enzymology, 217, 228□1257; U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

C. TAT376 or TAT377 Binding Organic Molecules

TAT binding organic molecules are organic molecules other than oligopeptides or antibodies as defined herein that bind, preferably specifically, to a TAT376 or TAT377 polypeptide as described herein. TAT376 or TAT377 binding organic molecules may be identified and chemically synthesized using known methodology (see, e.g., PCT Publication Nos. WO00/00823 and WO00/39585). TAT376 or TAT377 binding organic molecules are usually less than about 2000 daltons in size, alternatively less than about 1500, 750, 500, 250 or 200 daltons in size, wherein such organic molecules that are capable of binding, preferably specifically, to a TAT376 or TAT377 polypeptide as described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585). TAT376 or TAT377 binding organic molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

D. Screening for Anti-TAT376 or Anti-TAT377 Antibodies, TAT376 or TAT377 Binding Oligopeptides and TAT376 or TAT377 Binding Organic Molecules with the Desired Properties

Techniques for generating antibodies, oligopeptides and organic molecules that bind to TAT376 or TAT377 polypeptides have been described above. One may further select antibodies, oligopeptides or other organic molecules with certain biological characteristics, as desired.

The growth inhibitory effects of an anti-TAT376 or anti-TAT377 antibody, oligopeptide or other organic molecule of the invention may be assessed by methods known in the art, e.g., using cells which express a TAT376 or TAT377 polypeptide either endogenously or following transfection with the TAT376 or TAT377 gene. For example, appropriate tumor cell lines and TAT376- or TAT377-transfected cells may treated with an anti-TAT376 or anti-TAT377 monoclonal antibody, oligopeptide or other organic molecule of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an anti-TAT376 or anti-TAT377 antibody, TAT376 or TAT377 binding oligopeptide or TAT376 or TAT377 binding organic molecule of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. Preferably, the tumor cell is one that overexpresses a TAT376 or TAT377 polypeptide. Preferably, the anti-TAT376 or anti-TAT377 antibody, TAT376 or TAT377 binding oligopeptide or TAT376 or TAT377 binding organic molecule will inhibit cell proliferation of a TAT376- or TAT377-expressing tumor cell in vitro or in vivo by about 25-100% compared to the untreated tumor cell, more preferably, by about 30-100%, and even more preferably by about 50-100% or 70-100%, in one embodiment, at an antibody concentration of about 0.5 to 30 μg/ml. Growth inhibition can be measured at an antibody concentration of about 0.5 to 30 μg/ml or about 0.5 nM to 200 nM in cell culture, where the growth inhibition is determined 1-10 days after exposure of the tumor cells to the antibody. The antibody is growth inhibitory in vivo if administration of the anti-TAT376 or anti-TAT377 antibody at about 1 μg/kg to about 100 mg/kg body weight results in reduction in tumor size or reduction of tumor cell proliferation within about 5 days to 3 months from the first administration of the antibody, preferably within about 5 to 30 days.

To select for an anti-TAT376 or anti-TAT377 antibody, TAT376 or TAT377 binding oligopeptide or TAT376 or TAT377 binding organic molecule which induces cell death, loss of membrane integrity as indicated by, e.g., propidium iodide (PI), trypan blue or 7AAD uptake may be assessed relative to control. A PI uptake assay can be performed in the absence of complement and immune effector cells. TAT376 or TAT377 polypeptide-expressing tumor cells are incubated with medium alone or medium containing the appropriate anti-TAT376 or anti-TAT377 antibody (e.g, at about 10 μg/ml), TAT376 or TAT377 binding oligopeptide or TAT376 or TAT377 binding organic molecule. The cells are incubated for a 3 day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those anti-TAT376 or anti-TAT377 antibodies, TAT376 or TAT377 binding oligopeptides or TAT376 or TAT377 binding organic molecules that induce statistically significant levels of cell death as determined by PI uptake may be selected as cell death-inducing anti-TAT376 or anti-TAT377 antibodies, TAT376 or TAT377 binding oligopeptides or TAT376 or TAT377 binding organic molecules.

To screen for antibodies, oligopeptides or other organic molecules which bind to an epitope on a TAT376 or TAT377 polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody, oligopeptide or other organic molecule binds the same site or epitope as a known anti-TAT376 or anti-TAT377 antibody. Alternatively, or additionally, epitope mapping can be performed by methods known in the art. For example, the antibody sequence can be mutagenized such as by alanine scanning, to identify contact residues. The mutant antibody is initailly tested for binding with polyclonal antibody to ensure proper folding. In a different method, peptides corresponding to different regions of a TAT376 or TAT377 polypeptide can be used in competition assays with the test antibodies or with a test antibody and an antibody with a characterized or known epitope.

E. Antibody Dependent Enzyme Mediated Prodrug Therapy (ADEPT)

The antibodies of the present invention may also be used in ADEPT by conjugating the antibody to a prodrug-activating enzyme which converts a prodrug (e.g., a peptidyl chemotherapeutic agent, see WO81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328:457-458 (1987)). Antibody-abzyme conjugates can be prepared as described herein for delivery of the abzyme to a tumor cell population.

The enzymes of this invention can be covalently bound to the anti-TAT376 or anti-TAT377 antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature 312:604-608 (1984).

F. Full-Length TAT376 or TAT377 Polypeptides

The present invention also provides newly identified and isolated nucleotide sequences encoding polypeptides referred to in the present application as TAT376 or TAT377 polypeptides. In particular, cDNAs (partial and full-length) encoding various TAT376 or TAT377 polypeptides have been identified and isolated, as disclosed in further detail in the Examples below.

As disclosed in the Examples below, various cDNA clones have been deposited with the ATCC. The actual nucleotide sequences of those clones can readily be determined by the skilled artisan by sequencing of the deposited clone using routine methods in the art. The predicted amino acid sequence can be determined from the nucleotide sequence using routine skill. For the TAT376 or TAT377 polypeptides and encoding nucleic acids described herein, in some cases, Applicants have identified what is believed to be the reading frame best identifiable with the sequence information available at the time.

G. Anti-TAT376 or Anti-TAT377 Antibody and TAT376 or TAT377 Polypeptide Variants

In addition to the anti-TAT376 or anti-TAT377 antibodies and full-length native sequence TAT376 or TAT377 polypeptides described herein, it is contemplated that anti-TAT376 or anti-TAT377 antibody and TAT376 or TAT377 polypeptide variants can be prepared. Anti-TAT376 or anti-TAT377 antibody and TAT376 or TAT377 polypeptide variants can be prepared by introducing appropriate nucleotide changes into the encoding DNA, and/or by synthesis of the desired antibody or polypeptide. Those skilled in the art will appreciate that amino acid changes may alter post-translational processes of the anti-TAT376 or anti-TAT377 antibody or TAT376 or TAT377 polypeptide, such as changing the number or position of glycosylation sites or altering the membrane anchoring characteristics.

Variations in the anti-TAT376 or anti-TAT377 antibodies and TAT376 or TAT377 polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the antibody or polypeptide that results in a change in the amino acid sequence as compared with the native sequence antibody or polypeptide. Optionally the variation is by substitution of at least one amino acid with any other amino acid in one or more of the domains of the anti-TAT376 or anti-TAT377 antibody or TAT376 or TAT377 polypeptide. Guidance in determining which amino acid residue may be inserted, substituted or deleted without adversely affecting the desired activity may be found by comparing the sequence of the anti-TAT376 or TAT377 antibody or TAT376 or TAT377 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Anti-TAT376 or anti-TAT377 antibody and TAT376 or TAT377 polypeptide fragments are provided herein. Such fragments may be truncated at the N-terminus or C-terminus, or may lack internal residues, for example, when compared with a full length native antibody or protein. Certain fragments lack amino acid residues that are not essential for a desired biological activity of the anti-TAT376 or anti-TAT377 antibody or TAT376 or TAT377 polypeptide.

Anti-TAT376 or anti-TAT377 antibody and TAT376 or TAT377 polypeptide fragments may be prepared by any of a number of conventional techniques. Desired peptide fragments may be chemically synthesized. An alternative approach involves generating antibody or polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired antibody or polypeptide fragment, by polymerase chain reaction (PCR). Oligonucleotides that define the desired termini of the DNA fragment are employed at the 5′ and 3′ primers in the PCR. Preferably, anti-TAT376 or anti-TAT377 antibody and TAT376 or TAT377 polypeptide fragments share at least one biological and/or immunological activity with the native anti-TAT376 or anti-TAT377 antibody or TAT376 or TAT377 polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the anti-TAT376 or anti-TAT377 antibody or TAT376 or TAT377 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the anti-TAT376 or TAT377 antibody or TAT376 or TAT377 polypeptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the anti-TAT376 or TAT377 antibody or TAT376 or TAT377 polypeptide also may be substituted, generally with serine, to improve the oxidative stability of the molecule and prevent aberrant crosslinking. Conversely, cysteine bond(s) may be added to the anti-TAT376 or TAT377 antibody or TAT376 or TAT377 polypeptide to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human TAT376 or TAT377 polypeptide. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the anti-TAT376 or anti-TAT377 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-TAT376 or anti-TAT377 antibody.

H. Modifications of Anti-TAT376 or TAT377 Antibodies and TAT376 or TAT377 Polypeptides

Covalent modifications of anti-TAT376 or anti-TAT377 antibodies and TAT376 or TAT377 polypeptides are included within the scope of this invention. One type of covalent modification includes reacting targeted amino acid residues of an anti-TAT376 or anti-TAT377 antibody or TAT376 or TAT377 polypeptide with an organic derivatizing agent that is capable of reacting with selected side chains or the N- or C-terminal residues of the anti-TAT376 or anti-TAT377 antibody or TAT376 or TAT377 polypeptide. Derivatization with bifunctional agents is useful, for instance, for crosslinking anti-TAT376 or anti-TAT377 antibody or TAT376 or TAT377 polypeptide to a water-insoluble support matrix or surface for use in the method for purifying anti-TAT376 or anti-TAT377 antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the anti-TAT376 or anti-TAT377 antibody or TAT376 or TAT377 polypeptide included within the scope of this invention comprises altering the native glycosylation pattern of the antibody or polypeptide. “Altering the native glycosylation pattern” is intended for purposes herein to mean deleting one or more carbohydrate moieties found in native sequence anti-TAT376 or anti-TAT377 antibody or TAT376 or TAT377 polypeptide (either by removing the underlying glycosylation site or by deleting the glycosylation by chemical and/or enzymatic means), and/or adding one or more glycosylation sites that are not present in the native sequence anti-TAT376 or anti-TAT377 antibody or TAT376 or TAT377 polypeptide. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the anti-TAT376 or anti-TAT377 antibody or TAT376 or TAT377 polypeptide is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original anti-TAT376 or anti-TAT377 antibody or TAT376 or TAT377 polypeptide (for O-linked glycosylation sites). The anti-TAT376 or anti-TAT377 antibody or TAT376 or TAT377 polypeptide amino acid sequence may optionally be altered through changes at the DNA level, particularly by mutating the DNA encoding the anti-TAT376 or anti-TAT377 antibody or TAT376 or TAT377 polypeptide at preselected bases such that codons are generated that will translate into the desired amino acids.

Another means of increasing the number of carbohydrate moieties on the anti-TAT376 or anti-TAT377 antibody or TAT376 or TAT377 polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the anti-TAT376 or anti-TAT377 antibody or TAT376 or TAT377 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of anti-TAT376 or anti-TAT377 antibody or TAT376 or TAT377 polypeptide comprises linking the antibody or polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. The antibody or polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The anti-TAT376 or anti-TAT377 antibody or TAT376 or TAT377 polypeptide of the present invention may also be modified in a way to form chimeric molecules comprising an anti-TAT376 or anti-TAT377 antibody or TAT376 or TAT377 polypeptide fused to another, heterologous polypeptide or amino acid sequence.

In one embodiment, such a chimeric molecule comprises a fusion of the anti-TAT376 or anti-TAT377 antibody or TAT376 or TAT377 polypeptide with a tag polypeptide which provides an epitope to which an anti-tag antibody can selectively bind. The epitope tag is generally placed at the amino- or carboxyl-terminus of the anti-TAT376 or anti-TAT377 antibody or TAT376 or TAT377 polypeptide. The presence of such epitope-tagged forms of the anti-TAT376 or anti-TAT377 antibody or TAT376 or TAT377 polypeptide can be detected using an antibody against the tag polypeptide. Also, provision of the epitope tag enables the anti-TAT376 or anti-TAT377 antibody or TAT376 or TAT377 polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc Natl. Acad. Sci. USA, 87:6393-6397 (1990)].

In an alternative embodiment, the chimeric molecule may comprise a fusion of the anti-TAT376 or anti-TAT377 antibody or TAT376 or TAT377 polypeptide with an immunoglobulin or a particular region of an imrnunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of an anti-TAT376 or anti-TAT377 antibody or TAT376 or TAT377 polypeptide in place of at least one variable region within an Ig molecule. In a particularly preferred embodiment, the immunoglobulin fusion includes the hinge, CH₂ and CH₃, or the hinge, CH₁, CH₂ and CH₃ regions of an IgG1 molecule. For the production of immunoglobulin fusions see also U.S. Pat. No. 5,428,130 issued Jun. 27, 1995.

I. Preparation of Anti-TAT376 or Anti-TAT377 Antibodies and TAT376 or TAT377 Polypeptides

The description below relates primarily to production of anti-TAT376 or anti-TAT377 antibodies and TAT376 or TAT377 polypeptides by culturing cells transformed or transfected with a vector containing anti-TAT376 or anti-TAT377 antibody- and TAT376 or TAT377 polypeptide-encoding nucleic acid. It is, of course, contemplated that alternative methods, which are well known in the art, may be employed to prepare anti-TAT376 or anti-TAT377 antibodies and TAT376 or TAT377 polypeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the anti-TAT376 or anti-TAT377 antibody or TAT376 or TAT377 polypeptide may be chemically synthesized separately and combined using chemical or enzymatic methods to produce the desired anti-TAT376 or anti-TAT377 antibody or TAT376 or TAT377 polypeptide.

1. Isolation of DNA Encoding Anti-TAT376 or Anti-TAT377 Antibody or TAT376 or TAT377 Polypeptide

DNA encoding anti-TAT376 or anti-TAT377 antibody or TAT376 or TAT377 polypeptide may be obtained from a cDNA library prepared from tissue believed to possess the anti-TAT376 or anti-TAT377 antibody or TAT376 or TAT377 polypeptide mRNA and to express it at a detectable level. Accordingly, human anti-TAT376 or anti-TAT377 antibody or TAT376 or TAT377 polypeptide DNA can be conveniently obtained from a cDNA library prepared from human tissue. The anti-TAT376 or anti-TAT377 antibody- or TAT376 or TAT377 polypeptide-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding anti-TAT376 or anti-TAT377 antibody or TAT376 or TAT377 polypeptide is to use PCR methodology [Sambrook et al., supra; Dieffenbach et al., PCR Primer: A Laboratorv Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for anti-TAT376 or anti-TAT377 antibody or TAT376 or TAT377 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation regio (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g, in CHO cells.

In addition to prokaryotes, eukaryotic microbes such as filamentous fungi or yeast are suitable cloning or expression hosts for anti-TAT376 or anti-TAT377 antibody- or TAT376 or TAT377 polypeptide-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated anti-TAT376 or anti-TAT377 antibody or TAT376 or TAT377 polypeptide are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-TAT376 or anti-TAT377 antibody or TAT376 or TAT377 polypeptide production and cultured in conventional nutrient media modified as appropriate for inducing promoters, selecting transformants, or amplifying the genes encoding the desired sequences.

3. Selection and Use of a Replicable Vector

The nucleic acid (e.g., cDNA or genomic DNA) encoding anti-TAT376 or anti-TAT377 antibody or TAT376 or TAT377 polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The TAT376 or TAT377 may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the anti-TAT376 or anti-TAT377 antibody- or TAT376 or TAT377 polypeptide-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromycesα-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the anti-TAT376 or anti-TAT377 antibody- or TAT376 or TAT377 polypeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the anti-TAT376 or anti-TAT377 antibody- or TAT376 or TAT377 polypeptide-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding anti-TAT antibody or TAT376 or TAT377 polypeptide.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Anti-TAT376 or anti-TAT377 antibody or TAT376 or TAT377 polypeptide transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the anti-TAT376 or anti-TAT377 antibody or TAT376 or TAT377 polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the anti-TAT376 or anti-TAT377 antibody or TAT376 or TAT377 polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-TAT376 or anti-TAT377 antibody or TAT376 or TAT377 polypeptide.

Still other methods, vectors, and host cells suitable for adaptation to the synthesis of anti-TAT376 or anti-TAT377 antibody or TAT376 or TAT377 polypeptide in recombinant vertebrate cell culture are described in Gething et al., Nature, 293:620-625 (1981); Mantei et al., Nature, 281:40-46 (1979); EP 117,060; and EP 117,058.

4. Culturing the Host Cells

The host cells used to produce the anti-TAT376 or anti-TAT377 antibody or TAT376 or TAT377 polypeptide of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence TAT376 or TAT377 polypeptide or against a synthetic peptide based on the DNA sequences provided herein or against exogenous sequence fused to TAT376 or TAT377 DNA and encoding a specific antibody epitope.

6. Purification of Anti-TAT376 or Anti-TAT377 Antibody and TAT376 or TAT377 Polypeptide

Forms of anti-TAT376 or anti-TAT377 antibody and TAT376 or TAT377 polypeptide may be recovered from culture medium or from host cell lysates. If membrane-bound, it can be released from the membrane using a suitable detergent solution (e.g. Triton-X 100) or by enzymatic cleavage. Cells employed in expression of anti-TAT376 or anti-TAT377 antibody and TAT376 or TAT377 polypeptide can be disrupted by various physical or chemical means, such as freeze-thaw cycling, sonication, mechanical disruption, or cell lysing agents.

It may be desired to purify anti-TAT376 or anti-TAT377 antibody and TAT376 or TAT377 polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the anti-TAT376 or anti-TAT377 antibody and TAT376 or TAT377 polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular anti-TAT376 or anti-TAT377 antibody or TAT376 or TAT377 polypeptide produced.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62:1-13 (1983)). Protein G is recommended for all mouse isotypes and for human γ3 (Guss et al., EMBO J. 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

J. Pharmaceutical Formulations

Therapeutic formulations of the anti-TAT376 or anti-TAT377 antibodies, TAT376 or TAT377 binding oligopeptides, TAT376 or TAT377 binding organic molecules and/or TAT376 or TAT377 polypeptides used in accordance with the present invention are prepared for storage by mixing the antibody, polypeptide, oligopeptide or organic molecule having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG). The antibody preferably comprises the antibody at a concentration of between 5-200 mg/ml, preferably between 10-100 mg/ml.

The formulations herein may also contain more than one active compound as necessary for the particular indication being treated, preferably those with complementary activities that do not adversely affect each other. For example, in addition to an anti-TAT376 or anti-TAT377 antibody, TAT376 or TAT377 binding oligopeptide, or TAT376 or TAT377 binding organic molecule, it may be desirable to include in the one formulation, an additional antibody, e.g., a second anti-TAT376 or anti-TAT377 antibody which binds a different epitope on the TAT376 or TAT377 polypeptide, or an antibody to some other target such as a growth factor that affects the growth of the particular cancer. Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

K. Diagnosis and Treatment with Anti-TAT376 or Anti-TAT377 Antibodies, TAT376 or TAT377 Binding Oligopeptides and TAT376 or TAT377 Binding Organic Molecules

To determine TAT376 or TAT377 expression in the cancer, various diagnostic assays are available. In one embodiment, TAT376 or TAT377 polypeptide overexpression may be analyzed by immunohistochemistry (IHC). Paraffin embedded tissue sections from a tumor biopsy may be subjected to the IHC assay and accorded a TAT376 or TAT377 protein staining intensity criteria as follows:

Score 0—no staining is observed or membrane staining is observed in less than 10% of tumor cells.

Score 1+—a faint/barely perceptible membrane staining is detected in more than 10% of the tumor cells. The cells are only stained in part of their membrane.

Score 2+—a weak to moderate complete membrane staining is observed in more than 10% of the tumor cells.

Score 3+—a moderate to strong complete membrane staining is observed in more than 10% of the tumor cells.

Those tumors with 0 or 1+ scores for TAT376 or TAT377 polypeptide expression may be characterized as not overexpressing TAT376 or TAT377, whereas those tumors with 2+ or 3+ scores may be characterized as overexpressing TAT376 or TAT377.

Alternatively, or additionally, FISH assays such as the INFORM® (sold by Ventana, Arizona) or PATHVISION® (Vysis, Illinois) may be carried out on formalin-fixed, paraffin-embedded tumor tissue to determine the extent (if any) of TAT376 or TAT377 overexpression in the tumor.

TAT376 or TAT377 overexpression or amplification may be evaluated using an in vivo diagnostic assay, e.g., by administering a molecule (such as an antibody, oligopeptide or organic molecule) which binds the molecule to be detected and is tagged with a detectable label (e.g., a radioactive isotope or a fluorescent label) and externally scanning the patient for localization of the label.

As described above, the anti-TAT376 or anti-TAT377 antibodies, oligopeptides and organic molecules of the invention have various non-therapeutic applications. The anti-TAT376 or anti-TAT377 antibodies, oligopeptides and organic molecules of the present invention can be useful for diagnosis and staging of TAT376 or TAT377 polypeptide-expressing cancers (e.g., in radioimaging). The antibodies, oligopeptides and organic molecules are also useful for purification or immunoprecipitation of TAT376 or TAT377 polypeptide from cells, for detection and quantitation of TAT376 or TAT377 polypeptide in vitro, e.g., in an ELISA or a Western blot, to kill and eliminate TAT376- or TAT377-expressing cells from a population of mixed cells as a step in the purification of other cells.

Currently, depending on the stage of the cancer, cancer treatment involves one or a combination of the following therapies: surgery to remove the cancerous tissue, radiation therapy, and chemotherapy. Anti-TAT376 or anti-TAT377 antibody, oligopeptide or organic molecule therapy may be especially desirable in elderly patients who do not tolerate the toxicity and side effects of chemotherapy well and in metastatic disease where radiation therapy has limited usefulness. The tumor targeting anti-TAT376 or anti-TAT377 antibodies, oligopeptides and organic molecules of the invention are useful to alleviate TAT376- or TAT377-expressing cancers upon initial diagnosis of the disease or during relapse. For therapeutic applications, the anti-TAT376 or anti-TAT377 antibody, oligopeptide or organic molecule can be used alone, or in combination therapy with, e.g., hormones, antiangiogens, or radiolabelled compounds, or with surgery, cryotherapy, and/or radiotherapy. Anti-TAT376 or anti-TAT377 antibody, oligopeptide or organic molecule treatment can be administered in conjunction with other forms of conventional therapy, either consecutively with, pre- or post-conventional therapy. Chemotherapeutic drugs such as TAXOTERE® (docetaxel), TAXOL® (palictaxel), estramustine and mitoxantrone are used in treating cancer, in particular, in good risk patients. In the present method of the invention for treating or alleviating cancer, the cancer patient can be administered anti-TAT376 or anti-TAT377 antibody, oligopeptide or organic molecule in conjuction with treatment with the one or more of the preceding chemotherapeutic agents. In particular, combination therapy with palictaxel and modified derivatives (see, e.g., EP0600517) is contemplated. The anti-TAT376 or anti-TAT377 antibody, oligopeptide or organic molecule will be administered with a therapeutically effective dose of the chemotherapeutic agent. In another embodiment, the anti-TAT376 or anti-TAT377 antibody, oligopeptide or organic molecule is administered in conjunction with chemotherapy to enhance the activity and efficacy of the chemotherapeutic agent, e.g., paclitaxel. The Physicians' Desk Reference (PDR) discloses dosages of these agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

In one particular embodiment, a conjugate comprising an anti-TAT376 or anti-TAT377 antibody, oligopeptide or organic molecule conjugated with a cytotoxic agent is administered to the patient. Preferably, the immunoconjugate bound to the TAT376 or TAT377 protein is internalized by the cell, resulting in increased therapeutic efficacy of the immunoconjugate in killing the cancer cell to which it binds. In a preferred embodiment, the cytotoxic agent targets or interferes with the nucleic acid in the cancer cell. Examples of such cytotoxic agents are described above and include maytansinoids, calicheamicins, ribonucleases and DNA endonucleases.

The anti-TAT376 or anti-TAT377 antibodies, oligopeptides, organic molecules or toxin conjugates thereof are administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody, oligopeptide or organic molecule is preferred.

Other therapeutic regimens may be combined with the administration of the anti-TAT376 or anti-TAT377 antibody, oligopeptide or organic molecule. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the anti-TAT376 or anti-TAT377 antibody or antibodies, oligopeptides or organic molecules, with administration of an antibody directed against another tumor antigen associated with the particular cancer.

In another embodiment, the therapeutic treatment methods of the present invention involves the combined administration of an anti-TAT376 or anti-TAT377 antibody (or antibodies), oligopeptides or organic molecules and one or more chemotherapeutic agents or growth inhibitory agents, including co-administration of cocktails of different chemotherapeutic agents. Chemotherapeutic agents include estramustine phosphate, prednimustine, cisplatin, 5-fluorouracil, melphalan, cyclophosphamide, hydroxyurea and hydroxyureataxanes (such as paclitaxel and doxetaxel) and/or anthracycline antibiotics. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

The antibody, oligopeptide or organic molecule may be combined with an anti-hormonal compound; e.g., an anti-estrogen compound such as tamoxifen; an anti-progesterone such as onapristone (see, EP 616 812); or an anti-androgen such as flutamide, in dosages known for such molecules. Where the cancer to be treated is androgen independent cancer, the patient may previously have been subjected to anti-androgen therapy and, after the cancer becomes androgen independent, the anti-TAT376 or anti-TAT377 antibody, oligopeptide or organic molecule (and optionally other agents as described herein) may be administered to the patient.

Sometimes, it may be beneficial to also co-administer a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy, before, simultaneously with, or post antibody, oligopeptide or organic molecule therapy. Suitable dosages for any of the above co-administered agents are those presently used and may be lowered due to the combined action (synergy) of the agent and anti-TAT376 or anti-TAT377 antibody, oligopeptide or organic molecule.

For the prevention or treatment of disease, the dosage and mode of administration will be chosen by the physician according to known criteria. The appropriate dosage of antibody, oligopeptide or organic molecule will depend on the type of disease to be treated, as defined above, the severity and course of the disease, whether the antibody, oligopeptide or organic molecule is administered for preventive or therapeutic purposes, previous therapy, the patient's clinical history and response to the antibody, oligopeptide or organic molecule, and the discretion of the attending physician. The antibody, oligopeptide or organic molecule is suitably administered to the patient at one time or over a series of treatments. Preferably, the antibody, oligopeptide or organic molecule is administered by intravenous infusion or by subcutaneous injections. Depending on the type and severity of the disease, about 1 μg/kg to about 50 mg/kg body weight (e.g., about 0.1-15 mg/kg/dose) of antibody can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-TAT376 or anti-TAT377 antibody. However, other dosage regimens may be useful. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

Aside from administration of the antibody protein to the patient, the present application contemplates administration of the antibody by gene therapy. Such administration of nucleic acid encoding the antibody is encompassed by the expression “administering a therapeutically effective amount of an antibody”. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein.

The anti-TAT376 or anti-TAT377 antibodies of the invention can be in the different forms encompassed by the definition of “antibody” herein. Thus, the antibodies include full length or intact antibody, antibody fragments, native sequence antibody or amino acid variants, humanized, chimeric or fusion antibodies, immunoconjugates, and functional fragments thereof. In fusion antibodies an antibody sequence is fused to a heterologous polypeptide sequence. The antibodies can be modified in the Fc region to provide desired effector functions. As discussed in more detail in the sections herein, with the appropriate Fc regions, the naked antibody bound on the cell surface can induce cytotoxicity, e.g., via antibody-dependent cellular cytotoxicity (ADCC) or by recruiting complement in complement dependent cytotoxicity, or some other mechanism. Alternatively, where it is desirable to eliminate or reduce effector function, so as to minimize side effects or therapeutic complications, certain other Fc regions may be used.

In one embodiment, the antibody competes for binding or bind substantially to, the same epitope as the antibodies of the invention. Antibodies having the biological characteristics of the present anti-TAT376 or anti-TAT377 antibodies of the invention are also contemplated, specifically including the in vivo tumor targeting and any cell proliferation inhibition or cytotoxic characteristics.

Methods of producing the above antibodies are described in detail herein.

The present anti-TAT376 or anti-TAT377 antibodies, oligopeptides and organic molecules are useful for treating a TAT376- or TAT377-expressing cancer or alleviating one or more symptoms of the cancer in a mammal. Such a cancer includes prostate cancer, cancer of the urinary tract, lung cancer, breast cancer, colon cancer and ovarian cancer, more specifically, prostate adenocarcinoma, renal cell carcinomas, colorectal adenocarcinomas, lung adenocarcinomas, lung squamous cell carcinomas, and pleural mesothelioma. The cancers encompass metastatic cancers of any of the preceding. The antibody, oligopeptide or organic molecule is able to bind to at least a portion of the cancer cells that express TAT376 or TAT377 polypeptide in the mammal. In a preferred embodiment, the antibody, oligopeptide or organic molecule is effective to destroy or kill TAT376- or TAT377-expressing tumor cells or inhibit the growth of such tumor cells, in vitro or in vivo, upon binding to TAT376 or TAT377 polypeptide on the cell. Such an antibody includes a naked anti-TAT376 or anti-TAT377 antibody (not conjugated to any agent). Naked antibodies that have cytotoxic or cell growth inhibition properties can be further harnessed with a cytotoxic agent to render them even more potent in tumor cell destruction. Cytotoxic properties can be conferred to an anti-TAT376 or anti-TAT377 antibody by, e.g., conjugating the antibody with a cytotoxic agent, to form an immunoconjugate as described herein. The cytotoxic agent or a growth inhibitory agent is preferably a small molecule. Toxins such as calicheamicin or a maytansinoid and analogs or derivatives thereof, are preferable.

The invention provides a composition comprising an anti-TAT376 or anti-TAT377 antibody, oligopeptide or organic molecule of the invention, and a carrier. For the purposes of treating cancer, compositions can be administered to the patient in need of such treatment, wherein the composition can comprise one or more anti-TAT376 or anti-TAT377 antibodies present as an immunoconjugate or as the naked antibody. In a further embodiment, the compositions can comprise these antibodies, oligopeptides or organic molecules in combination with other therapeutic agents such as cytotoxic or growth inhibitory agents, including chemotherapeutic agents. The invention also provides formulations comprising an anti-TAT376 or anti-TAT377 antibody, oligopeptide or organic molecule of the invention, and a carrier. In one embodiment, the formulation is a therapeutic formulation comprising a pharmaceutically acceptable carrier.

Another aspect of the invention is isolated nucleic acids encoding the anti-TAT376 or anti-TAT377 antibodies. Nucleic acids encoding both the H and L chains and especially the hypervariable region residues, chains which encode the native sequence antibody as well as variants, modifications and humanized versions of the antibody, are encompassed.

The invention also provides methods useful for treating a TAT376 or TAT377 polypeptide-expressing cancer or alleviating one or more symptoms of the cancer in a mammal, comprising administering a therapeutically effective amount of an anti-TAT376 or anti-TAT377 antibody, oligopeptide or organic molecule to the mammal. The antibody, oligopeptide or organic molecule therapeutic compositions can be administered short term (acute) or chronic, or intermittent as directed by physician. Also provided are methods of inhibiting the growth of, and killing a TAT376 or TAT377 polypeptide-expressing cell.

The invention also provides kits and articles of manufacture comprising at least one anti-TAT376 or anti-TAT377 antibody, oligopeptide or organic molecule. Kits containing anti-TAT376 or anti-TAT377 antibodies, oligopeptides or organic molecules find use, e.g., for TAT376 or TAT377 cell killing assays, for purification or immunoprecipitation of TAT376 or TAT377 polypeptide from cells. For example, for isolation and purification of TAT376 or TAT377, the kit can contain an anti-TAT376 or anti-TAT377 antibody, oligopeptide or organic molecule coupled to beads (e.g., sepharose beads). Kits can be provided which contain the antibodies, oligopeptides or organic molecules for detection and quantitation of TAT376 or TAT377 in vitro, e.g., in an ELISA or a Western blot. Such antibody, oligopeptide or organic molecule useful for detection may be provided with a label such as a fluorescent or radiolabel.

L. Articles of Manufacture and Kits

Another embodiment of the invention is an article of manufacture containing materials useful for the treatment of anti-TAT376 or anti-TAT377 expressing cancer. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the cancer condition and may have a sterile access port (for example the container may be an intravenous solution bag or a vial having a stopper pierceable by a hypodermic injection needle). At least one active agent in the composition is an anti-TAT376 or anti-TAT377 antibody, oligopeptide or organic molecule of the invention. The label or package insert indicates that the composition is used for treating cancer. The label or package insert will further comprise instructions for administering the antibody, oligopeptide or organic molecule composition to the cancer patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for TAT376- or TAT377-expressing cell killing assays, for purification or immunoprecipitation of TAT376 or TAT377 polypeptide from cells. For isolation and purification of TAT376 or TAT377 polypeptide, the kit can contain an anti-TAT376 or anti-TAT377 antibody, oligopeptide or organic molecule coupled to beads (e.g., sepharose beads). Kits can be provided which contain the antibodies, oligopeptides or organic molecules for detection and quantitation of TAT376 or TAT377 polypeptide in vitro, e.g., in an ELISA or a Western blot. As with the article of manufacture, the kit comprises a container and a label or package insert on or associated with the container. The container holds a composition comprising at least one anti-TAT376 or anti-TAT377 antibody, oligopeptide or organic molecule of the invention. Additional containers may be included that contain, e.g., diluents and buffers, control antibodies. The label or package insert may provide a description of the composition as well as instructions for the intended in vitro or diagnostic use.

M. Uses for TAT376 or TAT377 Polypeptides and TAT376- or TAT377-Polypeptide Encoding Nucleic Acids

Nucleotide sequences (or their complement) encoding TAT376 or TAT377 polypeptides have various applications in the art of molecular biology, including uses as hybridization probes, in chromosome and gene mapping and in the generation of anti-sense RNA and DNA probes. TAT376- or TAT377-encoding nucleic acid will also be useful for the preparation of TAT376 or TAT377 polypeptides by the recombinant techniques described herein, wherein those TAT376 or TAT377 polypeptides may find use, for example, in the preparation of anti-TAT376 or anti-TAT377 antibodies as described herein.

The full-length native sequence TAT376 or TAT377 gene, or portions thereof, may be used as hybridization probes for a cDNA library to isolate the full-length TAT376 or TAT377 cDNA or to isolate still other cDNAs (for instance, those encoding naturally-occurring variants of TAT376 or TAT377 or TAT376 or TAT377 from other species) which have a desired sequence identity to the native TAT376 or TAT377 sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including promoters, enhancer elements and introns of native sequence TAT376 or TAT377. By way of example, a screening method will comprise isolating the coding region of the TAT376 or TAT377 gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the TAT376 or TAT377 gene of the present invention can be used to screen libraries of human cDNA, genomic DNA or mRNA to determine which members of such libraries the probe hybridizes to. Hybridization techniques are described in further detail in the Examples below. Any EST sequences disclosed in the present application may similarly be employed as probes, using the methods disclosed herein.

Other useful fragments of the TAT376- or TAT377-encoding nucleic acids include antisense or sense oligonucleotides comprising a singe-stranded nucleic acid sequence (either RNA or DNA) capable of binding to target TAT376 or TAT377 mRNA (sense) or TAT376 or TAT377 DNA (antisense) sequences. Antisense or sense oligonucleotides, according to the present invention, comprise a fragment of the coding region of TAT376 or TAT377 DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. Such methods are encompassed by the present invention. The antisense oligonucleotides thus may be used to block expression of TAT376 or TAT377 proteins, wherein those TAT376 or TAT377 proteins may play a role in the induction of cancer in mammals. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Preferred intragenic sites for antisense binding include the region incorporating the translation initiation/start codon (5′-AUG/5′-ATG) or termination/stop codon (5′-UAA, 5′-UAG and 5-UGA/5′-TAA, 5′-TAG and 5′-TGA) of the open reading frame (ORF) of the gene. These regions refer to a portion of the mRNA or gene that encompasses from about 25 to about 50 contiguous nucleotides in either direction (i.e., 5′ or 3′) from a translation initiation or termination codon. Other preferred regions for antisense binding include: introns; exons; intron-exon junctions; the open reading frame (ORF) or “coding region,” which is the region between the translation initiation codon and the translation termination codon; the 5′ cap of an mRNA which comprises an N7-methylated guanosine residue joined to the 5′-most residue of the mRNA via a 5′-5′ triphosphate linkage and includes 5′ cap structure itself as well as the first 50 nucleotides adjacent to the cap; the 5′ untranslated region (5′UTR), the portion of an mRNA in the 5′ direction from the translation initiation codon, and thus including nucleotides between the 5′ cap site and the translation initiation codon of an mRNA or corresponding nucleotides on the gene; and the 3′ untranslated region (3′UTR), the portion of an mRNA in the 3′ direction from the translation termination codon, and thus including nucleotides between the translation termination codon and 3′ end of an mRNA or corresponding nucleotides on the gene.

Specific examples of preferred antisense compounds useful for inhibiting expression of TAT376 or TAT377 proteins include oligonucleotides containing modified backbones or non-natural internucleoside linkages. Oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides. Preferred modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverte polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included. Representative United States patents that teach the preparation of phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Representative United States patents that teach the preparation of such oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In other preferred antisense oligonucleotides, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Preferred antisense oligonucleotides incorporate phosphorothioate backbones and/or heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] described in the above referenced U.S. Pat. No. 5,489,677, and the amide backbones of the above referenced U.S. Pat. No. 5,602,240. Also preferred are antisense oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-alkyl, S-alkyl, or N-alkyl; O-alkenyl, S-alkeynyl, or N-alkenyl; O-alkynyl, S-alkynyl or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred antisense oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O—(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂).

A further prefered modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—), group bridging the 2′ oxygen atom and the 4′ carbon atom wherein n is 1 or 2. LNAs and preparation thereof are described in WO 98/39352 and WO 99/14226.

Other preferred modifications include 2′-methoxy (2′-O—CH₃), 2′-aminopropoxy (2′-OCH₂CH₂CH₂ NH₂), 2′-allyl (2′—CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃ or —CH₂—C≡CH—) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H -pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi et al, Antisense Research and Applications, CRC Press, Boca Raton, 1993, pp. 276-278) and are preferred base substitutions, even more particularly when combined with 2′-O-methoxyethyl sugar modifications. Representative United States patents that teach the preparation of modified nucleobases include, but are not limited to: U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941 and 5,750,692, each of which is herein incorporated by reference.

Another modification of antisense oligonucleotides chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, cation lipids, phospholipids, cationic phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) and U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Chimeric antisense compounds of the invention may be formed as composite structures of two or more oligonucleotides, modified oligonucleotides, oligonucleosides and/or oligonucleotide mimetics as described above. Preferred chimeric antisense oligonucleotides incorporate at least one 2′ modified sugar (preferably 2′-O—(CH₂)₂—O—CH₃) at the 3′ terminal to confer nuclease resistance and a region with at least 4contiguous 2′-H sugars to confer RNase H activity. Such compounds have also been referred to in the art as hybrids or gapmers. Preferred gapmers have a region of 2′ modified sugars (preferably 2′-O—(CH₂)₂—O—CH₃) at the 3′-terminal and at the 5′ terminal separated by at least one region having at least 4 contiguous 2′-H sugars and preferably incorporate phosphorothioate backbone linkages. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

The probes may also be employed in PCR techniques to generate a pool of sequences for identification of closely related TAT376 or TAT377 coding sequences.

Nucleotide sequences encoding a TAT376 or TAT377 can also be used to construct hybridization probes for mapping the gene which encodes that TAT376 or TAT377 and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for TAT376 or TAT377 encode a protein which binds to another protein (example, where the TAT376 or TAT377 is a receptor), the TAT376 or TAT377 can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor TAT376 or TAT377 can be used to isolate correlative ligand(s). Screening assays can be designed to find lead compounds that mimic the biological activity of a native TAT376 or TAT377 or a receptor for TAT376 or TAT377. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode TAT376 or TAT377 or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding TAT376 or TAT377 can be used to clone genomic DNA encoding TAT376 or TAT377 in accordance with established techniques and the genomic sequences used to generate transgenic animals that contain cells which express DNA encoding TAT376 or TAT377. Methods for generating transgenic animals, particularly animals such as mice or rats, have become conventional in the art and are described, for example, in U.S. Pat. Nos. 4,736,866 and 4,870,009. Typically, particular cells would be targeted for TAT376 or TAT377 transgene incorporation with tissue-specific enhancers. Transgenic animals that include a copy of a transgene encoding TAT376 or TAT377 introduced into the germ line of the animal at an embryonic stage can be used to examine the effect of increased expression of DNA encoding TAT376 or TAT377. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of TAT376 or TAT377 can be used to construct a TAT376 or TAT377 “knock out” animal which has a defective or altered gene encoding TAT376 or TAT377 as a result of homologous recombination between the endogenous gene encoding TAT376 or TAT377 and altered genomic DNA encoding TAT376 or TAT377 introduced into an embryonic stem cell of the animal. For example, cDNA encoding TAT376 or TAT377 can be used to clone genomic DNA encoding TAT376 or TAT377 in accordance with established techniques. A portion of the genomic DNA encoding TAT376 or TAT377 can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the TAT376 or TAT377 polypeptide.

Nucleic acid encoding the TAT376 or TAT377 polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The nucleic acid molecules encoding the TAT376 or TAT377 polypeptides or fragments thereof described herein are useful for chromosome identification. In this regard, there exists an ongoing need to identify new chromosome markers, since relatively few chromosome marking reagents, based upon actual sequence data are presently available. Each TAT376 or TAT377 nucleic acid molecule of the present invention can be used as a chromosome marker.

The TAT376 or TAT377 polypeptides and nucleic acid molecules of the present invention may also be used diagnostically for tissue typing, wherein the TAT376 or TAT377 polypeptides of the present invention may be differentially expressed in one tissue as compared to another, preferably in a diseased tissue as compared to a normal tissue of the same tissue type. TAT376 or TAT377 nucleic acid molecules will find use for generating probes for PCR, Northern analysis, Southern analysis and Western analysis.

This invention encompasses methods of screening compounds to identify those that mimic the TAT376 or TAT377 polypeptide (agonists) or prevent the effect of the TAT376 or TAT377 polypeptide (antagonists). Screening assays for antagonist drug candidates are designed to identify compounds that bind or complex with the TAT376 or TAT377 polypeptides encoded by the genes identified herein, or otherwise interfere with the interaction of the encoded polypeptides with other cellular proteins, including e.g., inhibiting the expression of TAT376 or TAT377 polypeptide from cells. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a TAT376 or TAT377 polypeptide encoded by a nucleic acid identified herein under conditions and for a time sufficient to allow these two components to interact.

In binding assays, the interaction is binding and the complex formed can be isolated or detected in the reaction mixture. In a particular embodiment, the TAT376 or TAT377 polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the TAT376 or TAT377 polypeptide and drying. Alternatively, an immobilized antibody, e.g., a monoclonal antibody, specific for the TAT376 or TAT377 polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does. not bind to a particular TAT376 or TAT377 polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a TAT376 or TAT377 polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the TAT376 or TAT377 polypeptide may be added to a cell along with the compound to be screened for a particular activity and the ability of the compound to inhibit the activity of interest in the presence of the TAT376 or TAT377 polypeptide indicates that the compound is an antagonist to the TAT376 or TAT377 polypeptide. Alternatively, antagonists may be detected by combining the TAT376 or TAT377 polypeptide and a potential antagonist with membrane-bound TAT376 or TAT377 polypeptide receptors or recombinant receptors under appropriate conditions for a competitive inhibition assay. The TAT376 or TAT377 polypeptide can be labeled, such as by radioactivity, such that the number of TAT376 or TAT377 polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the TAT376 or TAT377 polypeptide and a cDNA library created from this RNA is divided into pools and used to transfect COS cells or other cells that are not responsive to the TAT376 or TAT377 polypeptide. Transfected cells that are grown on glass slides are exposed to labeled TAT376 or TAT377 polypeptide. The TAT376 or TAT377 polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled TAT376 or TAT377 polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled TAT376 or TAT377 polypeptide in the presence of the candidate compound. The ability of the compound to enhance or block this interaction could then be measured.

More specific examples of potential antagonists include an oligonucleotide that binds to the fusions of immunoglobulin with TAT376 or TAT377 polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the TAT376 or TAT377 polypeptide that recognizes the receptor but imparts no effect, thereby competitively inhibiting the action of the TAT376 or TAT377 polypeptide.

Another potential TAT376 or TAT377 polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature TAT376 or TAT377 polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the TAT376 or TAT377 polypeptide. The antisense RNA oligonucleotide hybridizes to the mRNA in vivo and blocks translation of the mRNA molecule into the TAT376 or TAT377 polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the TAT376 or TAT377 polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the TAT376 or TAT377 polypeptide, thereby blocking the normal biological activity of the TAT376 or TAT377 polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

Isolated TAT376 or TAT377 polypeptide-encoding nucleic acid can be used herein for recombinantly producing TAT376 or TAT377 polypeptide using techniques well known in the art and as described herein. In turn, the produced TAT376 or TAT377 polypeptides can be employed for generating anti-TAT376 or anti-TAT377 antibodies using techniques well known in the art and as described herein.

Antibodies specifically binding a TAT376 or TAT377 polypeptide identified herein, as well as other molecules identified by the screening assays disclosed hereinbefore, can be administered for the treatment of various disorders, including cancer, in the form of pharmaceutical compositions.

If the TAT376 or TAT377 polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al, Proc. Natl. Acad. Sci. USA, 90: 7889-7893 (1993).

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Tissue Expression Profiling Using GeneExpress®

1.1 GeneExpress®, Gene Logic Inc. Gene Expression Studies

A proprietary database containing gene expression information (GeneExpress®, Gene Logic Inc., Gaithersburg, Md.) was analyzed in an attempt to identify polypeptides (and their encoding nucleic acids) whose expression is significantly upregulated in a particular tumor tissue(s) of interest as compared to other tumor(s) and/or normal tissues. Specifically, analysis of the GeneExpress® database was conducted using either software available through Gene Logic Inc., Gaithersburg, Md., for use with the GeneExpress® database or with proprietary software written and developed at Genentech, Inc. for use with the GeneExpress® database. The rating of positive hits in the analysis is based upon several criteria including, for example, tissue specificity, tumor specificity and expression level in normal essential and/or normal proliferating tissues. The following is a list of molecules whose tissue expression profile as determined from an analysis of the GeneExpress® database evidences high tissue expression and significant upregulation of expression in a specific tumor or tumors as compared to other tumor(s) and/or normal tissues and optionally relatively low expression in normal essential and/or normal proliferating tissues. As such, the molecules listed below are excellent polypeptide targets for the diagnosis and therapy of cancer in mammals.

| | upregulation of |
|---|---|
| | expression in: as |
| Molecule | compared to: |
| DNA327307 (TAT376) | breast tumor |
| DNA327307 (TAT376) | normal breast tissue |
| | colon tumor |
| DNA327307 (TAT376) | normal colon tissue |
| | rectum tumor |
| DNA327307 (TAT376) | normal rectum tissue |
| | uterine tumor |
| DNA327307 (TAT376) | normal uterine tissue |
| | esophagus tumor |
| DNA327307 (TAT376) | normal esophagus |
| | tissue |
| | lung tumor |
| DNA327307 (TAT376) | normal lung tissue |
| | ovarian tumor |
| DNA327307 (TAT376) | normal ovarian |
| | tissue |
| | pancreatic tumor |
| DNA327307 (TAT376) | normal pancreatic |
| | tissue |
| | stomach tumor |
| DNA327307 (TAT376) | normal stomach |
| | tissue |
| | breast tumor |
| DNA327308 (TAT377) | normal breast tissue |
| | colon tumor |
| DNA327308 (TAT377) | normal colon tissue |
| | rectum tumor |
| DNA327308 (TAT377) | normal rectum tissue |
| | uterine tumor |
| DNA327308 (TAT377) | normal uterine tissue |
| | esophagus tumor |
| DNA327308 (TAT377) | normal esophagus |
| | tissue |
| | lung tumor |
| DNA327308 (TAT377) | normal lung tissue |
| | ovarian tumor |
| DNA327308 (TAT377) | normal ovarian |
| | tissue |
| | pancreatic tumor |
| DNA327308 (TAT377) | normal pancreatic |
| | tissue |
| | stomach tumor |
| | normal stomach |
| | tissue |

1.2 Gene Logic Affymetrix® Oligonucleotide Microarray Studies

1.2.1 Gene Logic Affymetrix® Microarray studies

Further analysis was performed using the Gene Logic expression database for screening cancer-specific expression profiles in colorectal neoplasia (tissue sample n=7,579). The screen was conducted using algorithms to identify probesets showing stronger hybridization signals in CRC than normal human tissues. The specificity of all probeset sequences of interest was checked by BLASTn (NCBI). The Gene Logic data was also examined to identify contiguous probesets expressed at a similar level in each case of CRC (n=176) relative to normal pooled colonic mucosa samples (n=225).

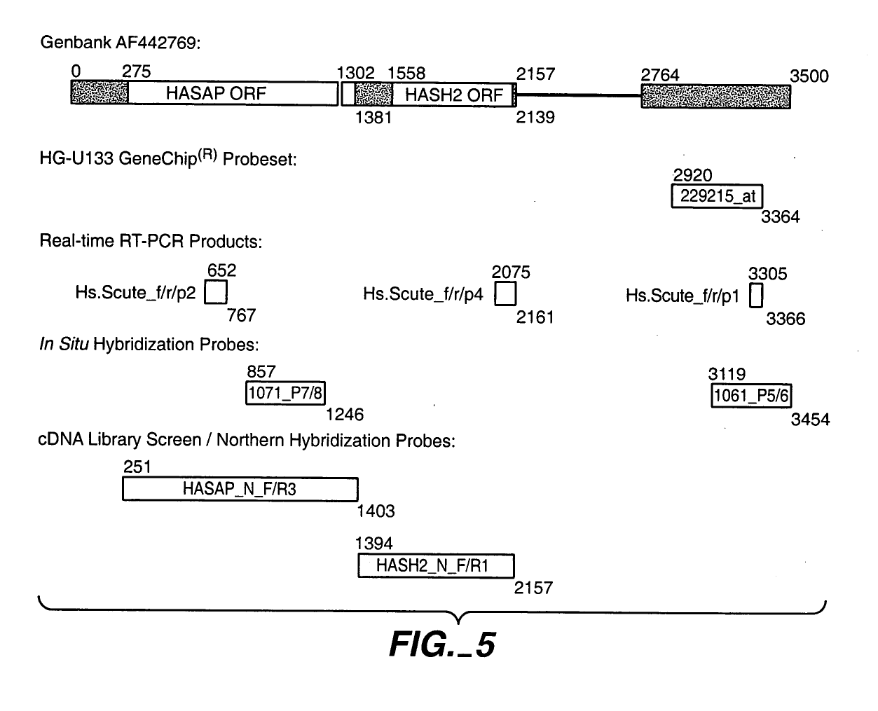

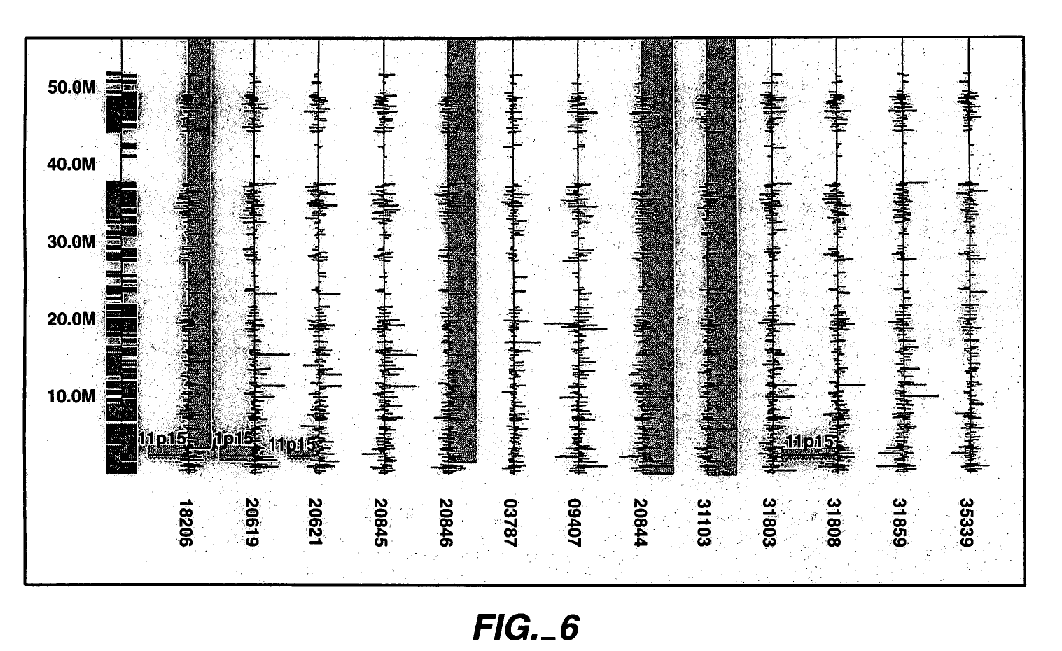

Two probes were identified [probe 229215_at (GeneChip HG-U133 and probe 89164_at (GeneChip HG-U95)] from the Gene Logic screen for cancer-specific expression profiles in colorectal neoplasia. Both probes were complementary to the second exon of the ASCL2 transcript (synonymous with TAT377 or DNA32708) shown in FIG. 5 and showed a strong correlation in signal intensity for 225 colorectal tissue RNA samples on both GeneChips (data not shown, R²=0.85). Probe 229215_at was selected for all further analysis. The median signal intensity was 5.4 fold greater in villous adenomas and in situ lesions (n=28), 8.8 fold greater in non-metastatic CRC (n=176) and 6.8 fold greater in metastatic CRC (n=46) than normal colon (n=270) (respecitively 267 vs. 49, p<0.0001; 431 vs. 49, p<0.0001; 334 vs. 49, p<0.001). Five carcinoid tumors of the ileum, appendix and colon showed a median 6 fold regulation compared to normal colon (288 vs. 48, range 3-29 fold). Adenomas from patients with familial adenomatous polyposis (4 small intestive, 4 colorectal) demonstrated a median 6 fold upregualtion compared to normal colon (292 vs. 48, range 5-10 fold). There was no significant difference in expression between the tumor types. Screening the Gene Logic colorectal data for contiguous probes expressed at a similar level in each case identified a cluster of probes at the 11p15.5 locus that are consistently upregulated (FIG. 6). Probe 229215_at was located within this cluster. 1.2.2 Murine Synteny

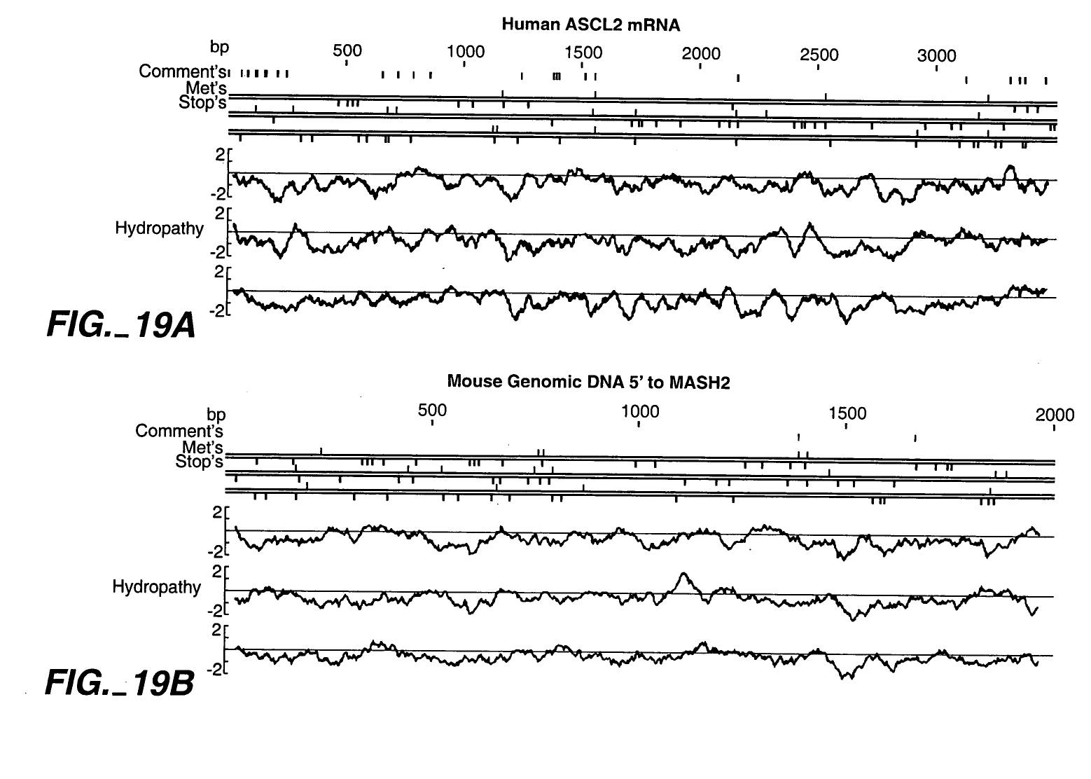

To validate the significance of the alleged HASAP open reading frame (ORF) in ASCL2, was performed on the predicted sequence (Genbank AF442769) and the corresponding murine genomic sequence (Genbank NT-039437.1) using proprietary software written and developed at Genentech, Inc. In addition, the position of start and stop codons was mapped in this region. The murine genomic sequence corresponding to the HASAP ORF shows 38.2% synteny with the human ORF (FIG. 19). However, the murine sequence is broken up with stop codons.

### Example 2

### NCBI Serial Analysis of Gene Expression (SAGE) Database and Incyte EST Database Searches

2.1 NCBI Serial Analysis of Gene Expression (SAGE) Database Search

In silico subtractive hybridization was performed on pooled CRC and normal colon SAGE libraries derived from the Cancer Genome Anatomy Project (Lash, A. E, Tolstoshev C M, Wagner L, Schuler G D, Strausberg R L, Riggins G J, et al. SAGEmap: a public gene expression resource. Genome Res 10(7):1051-60 (2000)) .HTTP://www.ncbi.nlm.nih.gov./SAGE/). Two normal colon SAGE libraries with a total of 99,772 tags (SAGE_NC1 and SAGE_NC2) were screened with xProfiler (NCBI) against six cell-line and primary CRC SAGE libraries with a total of 341,986 tags (SAGE_Caco_{—}2, SAGE_HCT116, SAGE_RKO, SAGE_SW837, SAGE_Tu102 and SAGE_Tu98). Tags expressed in CRC but not normal colon were screened for homology to GenBank AF442769. Profiling the SAGE libraries in the CGAP (SEQ ID NO:6) database identified a tag, as CTGGCCAAGA (SEQ ID NO:7) specific for ASCL2. The abundance in Caco_{—}2, Tu102 and Tu98 libraries was respectively 32, 17 and 163 tags per million. The tag was not present in either of the two normal colon libraries. 2.2 Incyte Expressed Sequence Tag (EST) Database Search

In silico subtractive hybridization was performed on pooled CRC and normal colon EST libraries derived from the LifeSeq Gold database (Incyte Genomics, Palo Alto, Calif.). Six (6) normal colon libraries with a total of 16,562 ESTs were screened against seventeen (17) primary CRC EST libraries with a total of 47,986 ESTs. ESTs over-expressed in CRC but not normal colon were screened for homology to AF442769. In silico subtractive hybridization of EST libraries in the LifeSeq database identified an EST (ID: 17090.3) with 98% sequence identity to ASCL2 and an absolute abundance of 1% in the CRC libraries.

### Example 3

### In Situ Hybridization

3.1 Tissue Culture

JEG3 and SW480 cell lines were obtained from the American Type Culture Collection (Manassas, Va.), all other cell lines were obtained from the National Cancer Institute (Bethesda, Md.). Cells were cultured according to the supplied protocols. 3.2 Primary Human Tissues and Tissue Microarray Construction

Tissue microarrays (TMAs) were constructed using a Beecher Instruments microarrayer (Silver Spring, Md.) as described by Kononen, J. et al., Nat Med, 4:(7):844-7 (1998); and Hoos, A. et al., Am J Pathol, 158(4):1245-51 (2001), using formalin-fixed paraffin-embedded (FFPE) tissues from the University of Michigan (Ann Arbor, Mich.). Haematoxylin and eosin (H&E) staining for verification of the histology was performed on the first section cut from each TMA block. TMA H2001-688 contained normal samples from the aorta, bladder, brain, breast, colon, heart, kidney, lung, lymph node, ovary, pancreas, placenta, prostate, seminal vesicles, skin, small intestine, spleen, stomach, testis, thyroid and tonsil. TMA H2002-223 represented 10 cell lines, 50 CRCs and 25 normal colon samples.

Additional TMAs representing 342 CRCs and matched normal mucosa from 330 patients were obtained from Dr. H. Grabsch (Academic Unit of Pathology, Leeds, UK) and Professor W. Mueller (Gemeinschaftspraxis Pathologie, Starnberg, Germany). All patients had undergone potentially curative resections at the Marien-Hospital (Duesseldorf, Germany) between January 1990 and December 1995. Survival data was collected on the cases, with a median follow-up time of 4.2 years. All tumors were staged and graded according to the World Health Organization criteria and without knowledge of the clinical outcome. In addition, an elastic van Gieson stain was used to detect vascular involvement.

3.3 Synthesis and Labeling of the Probe

cDNA probe templates were generated from CRC and placental marathon-ready cDNA (BD Clontech, Palo Alto, Calif.). Each 50 μl polymerase chain reaction (PCR) contained 0.5 ng of cDNA, 33 ng of each primer, 0.6 mM dNTPs (0.15 mM each of dATP, dCTP, dGTP, dTTP), 1× polymerase mix, 1× buffer and 1.0 M GC-Melt (BD Clontech). In the first round, primers 1071_P5/6 and 1061_P1/2 were used in PCR program 1 (see Table 7 below) to amplify fragments from the long and short transcripts respectively (FIG. 5). One microliter of each product was then used as a template in a nested PCR employing primers that included T7 and T3 RNA polymerase initiation sites, respectively 1071_P7/8 and 1061_P5/6 (see Table 8 below). β-actin template was synthesized in one round using 117_P3/4. For each probe to be synthesized, 12 μl (125 mCi) of [α³³P]-UTP (Amersham Biosciences, Piscataway, N.J.) was speed-vacuumed until dry. Each aliquot was reconstituted in 1× buffer, 4.5 mM dithiothreitol, 0.23 mM rNTPs (0.08 mM each rATP, rCTP, rGTP), 2.3 μM rUTP, 1.4 U/μl RNAse inhibitor, 0.05 μg/μl template, 0.7 U/μl of either T7 (sense probe) or T3 (anti-sense probe) RNA polymerase (Promega, Madison, Wis.). In vitro transcription took place over 1 hour at 37° C. Samples were then treated with 0.05 U/μl DNase (Promega) for 15 minutes at 37° C. and purified over RNeasy-mini columns (Qiagen, Valencia, Calif.). Probe activity was determined by scintillation counting. Denatured probe size was checked on a 2% agarose gel, exposed to Biomax MS film (Eastman Kodak, Rochester, N.Y.) for 1 hour.

TABLE 7
PCR Programs
Program 1 (Probe synthesis)

| Hot Start | 94° C. for 2 min |
|---|---|
| Denaturation | 94° C. for 30 sec |
| Annealing | 68° C. for 2 min |
| Cycles | 30 |

Program 2 (Real-time RT-PCR)

| | |
|---|---|
| Reverse Transcriptase | 48° C. for 30 min |
| Hot-Start | 95° C. for 10 min |
| Denaturation | 95° C. for 45 sec |
| Annealing | 60° C. for 1 min |
| Cycles | 40 |

Program 3 (Probe Synthesis/cDNA Cloning)

| | |
|---|---|
| Hot-Start | 94° C. for 3 min |
| Denaturation | 94° C. for 30 sec/94° C. for 30 sec |
| Annealing | 65° C. to 56° C. for 30 sec/55° C. for 30 sec |
| Elongation | 72° C. for 2 min/72° C. for 2 min |
| Cycles | 9/15 |
| Final Extension | 72° C. for 10 min |

Program 4 (Probe Synthesis)

| | |
|---|---|
| Hot-Start | 94° C. for 3 min |
| Denaturation | 94° C. for 30 sec |
| Annealing | 55° C. for 30 sec |
| Elongation | 72° C. for 2 min |
| Cycles | 15 |
| Final Extension | 72° C. for 10 min |

TABLE 8
Primer and probe sequences

| | | | |
|---|---|---|---|
| | Genbank | | |
| Purpose | Accession | Name | Sequence 5′ to 3′ |

| | | | | |
|---|---|---|---|---|
| Taqman | AF442769 | hs.scute_f1 | GGCACTGCTGCTCTGCTA | (SEQ ID NO:10) |
| | | | bp start 3306 bp finish 3323 | |
| | | hs.scute_f1 | GTTCACGCTCCCTTGAAGA | (SEQ ID NO:11) |
| | | | bp start 3367 bp finish 3349 | |
| | | hs.scute p1 | GAGAAGCCTGTGTGGGGCACA | (SEQ ID NO:12) |
| | | | bp start 3325 bp finish 3345 | |
| | | | 5′-FAM, 3′-TAMRA | |
| | | hs.scute_f2 | AGGAACCCAGC1TTGTFITAGC | (SEQ ID NO:13) |
| | | | bp start 653 bp finish 672 | |
| | | hs.scute_r2 | AGGGTCTCAGCCAATCGT | (SEQ ID NO:14) |
| | | | bp start 785 bp finish 768 | |
| | | hs.scute_p2 | CGTACGCGCTTCCTCAATGGG | (SEQ ID NO:15) |
| | | | bp start 700 bp finish 720 | |
| | | | 5′-FAM, 3′-TAMRA | |
| | | hs.scute_f4 | AAGGCGCGCTGAGTCCTGC | (SEQ ID NO:16) |
| | | | bp start 2075 bp finish 2093 | |
| | | hs.scute_r4 | TTACCTCATAGGTCGAGGGCGCT | (SEQ ID:17) |
| | | | bp start 2161 bp finish 2139 | |
| | | hs.scute_p4 | CGAGCTACTCGACTTCTCCAGCTG | (SEQ ID NO:18) |
| | | | bp start 2100 bp finish 2123 | |
| | | | 5′-FAM, 3′-TAMRA | |
| | NM_000981 | RPL19_f1 | GCGGATTCTCATGGAACACA | (SEQ ID NO:19) |
| | | | bp start 433 bp finish 452 | |
| | | RPL19_f1 | GCTCAGCCAGGAGCTTCTTG | (SEQ ID NO:20) |
| | | | bp start 500 bp finish 481 | |
| | | RPL19_p1 | CACAAGCTGAAGGCAGACAAGGCCC | (SEQ ID NO:21) |
| | | | bp start 455 bp finish 479 | |
| | | | 5′-FAM, 3′-TAMRA | |
| Cloning/ | AF442769 | HASAP_N_F1 | CACCGACGGGAAGCGAGA | (SEQ ID NO:22) |
| Probe Sythesis | | | bp start 211 bp finish 228 | |
| | | HASAP_N_R1 | CCTAGTGGTGGCAGGCCGTAC | (SEQ ID NO:23) |
| | | | bp start 1382 bp finish 1362 | |
| | | HASAP_N_F3 | GGCTGGAGGTGGGGGATACTG | (SEQ ID NO:24) |
| | | | bp start 251 bp finish 271 | |
| | | HASAP_N_R3 | CTGGAGCCCGGGGATAGGA | (SEQ ID NO:25) |
| | | | bp start 1511 bp finish 1491 | |
| | | HASH2_N_F1 | CGGGCTCCAGACGACCTAGGAC | (SEQ ID NO:26) |
| | | | bp start 1394 bp finish 1415 | |
| | | HASH2_N_R1 | CTCATAGGTCGAGGGCGCTCAGTA | (SEQ ID NO:27) |
| | | | bp start 2157 bp finish 2134 | |
| | | 327308_XhoI | AAAGGGAAACTCGAGATGGACGGC | (SEQ ID NO:28) |
| | | | GGCACACTGC | |
| | | | bp start 1558 bp finish 1576 | |

Includes XhoI Rx Site (bold)

| | | |
|---|---|---|
| 327308_HindIII | AAAGGGAAAAAGCTTGTAGCCCCCT | (SEQ ID NO:29) |
| | AACCAGCTGGAGAAGTCGAG | |
| | bpstart 2136 bp finish 2107 | |

Includes HindIII Rx Site (bold)

| | | |
|---|---|---|
| 327307_XhoI | AAAGGGAAACTCGAGATGGAGGGA | (SEQ ID NO:30) |
| | GCCACGGTGGG | |
| | bp start 275 bp finish 294 | |

Includes Xhol Rx Site (bold) 134

| | | |
|---|---|---|
| 327307 HindIII | AAAGGGAAAAAGCTTGTGGTGGCAG | (SEQ ID NO:31) |
| | GCCGTACGCG | |
| | bp start 1378 bp finish 1359 | |

Includes HindKIII Rx Site (bold)

| | | |
|---|---|---|
| 1061_P1 | CCCCACAGCTTCTCGAC | (SEQ ID NO:32) |
| | bp start 2987 bp finish 3003 | |
| 1061_P2 | AGCAGGGTTTGAGGAGTAGTG | (SEQ ID NO:33) |
| | bp start 3465 bp finish 3445 | |
| 1061_P5 | GGATTCTAATACGACTCACTATAG | (SEQ ID NO:34) |
| | GGCCTGGCAAACGGAGACCTATT | |
| | bp start 3119 bp finish 3138 | |

Includes T7 promoter (bold) 390

| | | |
|---|---|---|
| 1061_P6 | CTATGAAATTAACCCTCACTAAAG | (SEQ ID NO:35) |
| | GGAAGGAGTAGTGACCACGGAAGT | |
| 1071_P5 | CGCCCCGGGAACCTGAACCTC | (SEQ ID NO:36) |
| | bp start 456 bp finish 476 | |
| 1071_P6 | GCCGCCGTCGCCTTTCTCA | (SEQ ID NO:37) |
| | bp start 1245 bp finish 1227 | |
| 1071_P7 | GGATTCTAATACGACTCACTATAG | (SEQ ID NO:38) |
| | GGCATCTTGCGCGCCTCCCGAACA | |
| | bp start 857 bp finish 877 | |

Includes T7 promoter (bold)

| | | |
|---|---|---|
| 1071_P8 | CTATGAAATTAACCCTCACTAAAG | (SEQ ID NO:39) |
| | GGACGCCGCCGTCGCCTTTCTCA | |
| | bp start 1246 bp finish 1227 | |

Includes T3 promoter (bold)

| | | | |
|---|---|---|---|
| NM_001101 | β-actin_N_F1 | GCCGGGACCTGACTGAC | (SEQ ID NO:40) |
| | | bp start 618 bp finish 634 | |
| | β-actin_N_R1 | AACAAATAAAGCCATGCCAAT | (SEQ ID NO:41) |
| | | bp start 1291 bp finish 1271 | |
| | 117_P3 | GGATTCTAATACGACTCACTATAG | (SEQ ID NO:42) |
| | | GGCGCTGCCTGACGGCCAGGTC | |
| | | bp start 796 bp finish 814 | |

Includes T7 promoter (bold) 345

| | | |
|---|---|---|
| 117_P4 | CTATGAAATTAACCCTCACTAAAG | (SEQ ID NO:43) |
| | GGAGAGTACTTGCGCTCAGGAGGAG | |
| | bp start 1086 bp finish 1065 | |

Includes T3 promoter (bold)

| | | | | |
|---|---|---|---|---|
| Sequencing | U55762 | egfp.f1 | GCTATTACCATGGTGATG | (SEQ ID NO:44) |
| | | | bp start 353 bp finish 370 | |
| | | egfp.r1 | CCTTGAAGAAGATGGTG | (SEQ ID NO:45) |
| | | | bp start 985 bp finish 969 | |
| | | pRK.for | GTGAAATTTGTGATGCTATTG | (SEQ ID NO:46) |
| | | | bp start 1506 bp finish 1486 | |
| | | pRK.rev | TGCCTTTCTCTCCACAGG | (SEQ ID NO:47) |
| | | | bp start 856 bp finish 873 | |
| | AF442769 | 327308.f1 | GCAAGAAGCTGAGCAAG | (SEQ ID NO:48) |
| | | | bp start 1805 bp finish 18121 | |
| | U55762 | 327308.f10 | GGACTCAGATCTCGAGATG | (SEQ ID NO:49) |
| | | | bp start 603 bp finish insert | |
| | AF442769 | 327308.f11 | CGTGAAGCTGGTGAACTTG | (SEQ ID NO:50) |
| | | | bp start 1743 bp finish 1761 | |
| | | 327308.r1 | CCTTGCTCAGCTTCTTG | (SEQ ID NO:51) |
| | | | bp start 1822 bp finish 1806 | |
| | | 327308.r10 | CCTAACCAGCTGGAGAAGTC | (SEQ ID NO:52) |
| | | | bp start 2129 bp finish 2110 | |
| | | 327307.f1 | CCTGCGTACCTTGCTTTG | (SEQ ID NO:53) |
| | | | bp start 1009 bp finish 1026 | |
| | | 327307.f2 | CCTCAGTCTCGACCACTCC | (SEQ ID NO:54) |
| | | | bp start 485 bp finish 503 | |
| | | 327307.r1 | CCACCTGTGCGTTAATCTAC | (SEQ ID NO:55) |
| | | | bp start 571 bp finish 552 | |
| | | 327307.r2 | CCTCCTTCCTTCGCCTC | (SEQ ID NO:56) |
| | | | bp start 1223 bp finish 1207 | |
| | U55762 | 327307.r3 | CGTAGGTCAGGGTGGTCAC | (SEQ ID NO:57) |
| | | | bp start 880 bp finish 862 | |

BLASTn (NCB1, Bethesda, MD) was run on all primer and probe sequences to confirm their specificity.

3.4 Probe Hybridization and Washes

FFPE tissue sections (4 μm) were deparaffinized and treated with proteinase K (20 μm/ml in 2×SSC) at 37° C. for 15 minutes. Each section was covered with 50 μl of hybridization buffer (10% dextran sulfate, 50% formamide, 2×SSC) and incubated for 1-4 hours at 68° C. For each slide to be hybridized, 1×10⁶ cpm of denatured probe and 50 μg tRNA were made in 50 μl of hybridization buffer, which was mixed with the pre-hybridization solution. Hybridization took place over 19 hours at 55° C. Slides were washed twice under low stringency conditions (10 minutes in 2×SSC and 0.01 M EDTA at room temperature). RNase-A treatment (20 μg/ml in 2×SSC) was carried out for 30 minutes at 37° C., prior to two further low stringency washes and one high stringency wash (2 hours in 1×SCC and 0.01 M EDTA at 55° C.). 3.5 Detection of Hybridized Probe and Analysis

Tissue sections were dehydrated, air-dried and exposed to a phosphorscreen for 18 hours at room temperature. Immediately post-incubation, the phosphorscreen was scanned with a Typhoon 9410 (Amersham Biosciences). Tissue autofluorescence was assessed by scanning at 532/610 nm (excitation/emission). Slides were then dipped in NBT2 nuclear track emulsion (Eastman Kodak), exposed for four weeks at 4° C., developed and counterstained with H&E. Background subtraction, griding and analysis of the IPs was undertaken with Phoretix Array v.3 (Nonlinear Dynamics, Newcastle upon Tyne, UK). Cores were scored +/− for the presence or absence of the hybridized probe.

3.6 Results

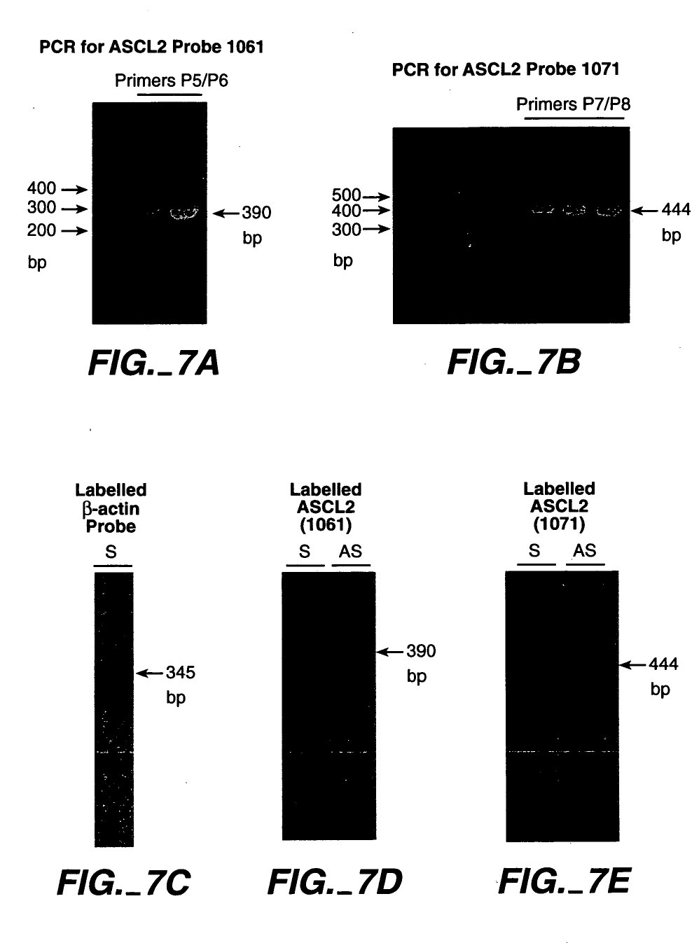

Histological review of tissue microarrays (TMAs) described above demonstrated that at least two or three cores were present and correctly diagnosed for all cases represented in H2002-223 and H2001-688. At least two of the three cores were present for 336 CRCs and at least one core for 191 normal mucosa samples in the Dusseldorf TMAs. All probes amplified and labeled specifically (FIG. 7).

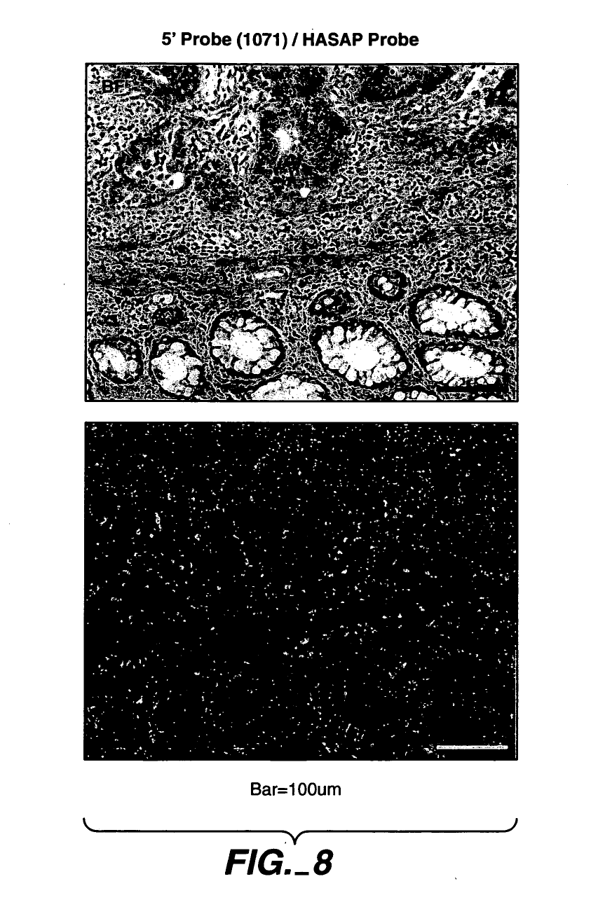

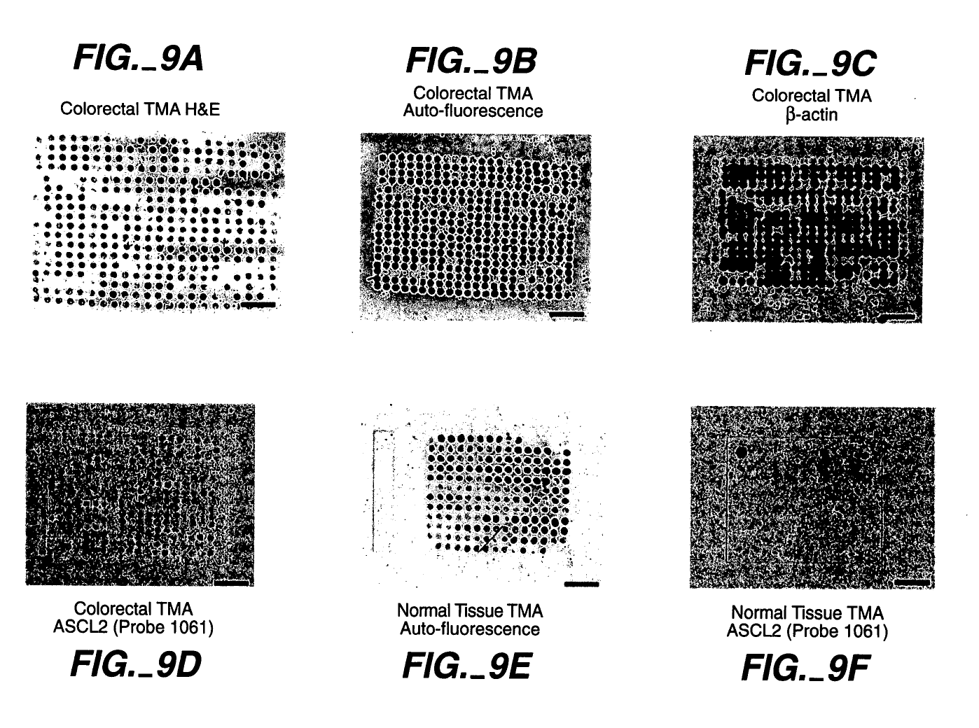

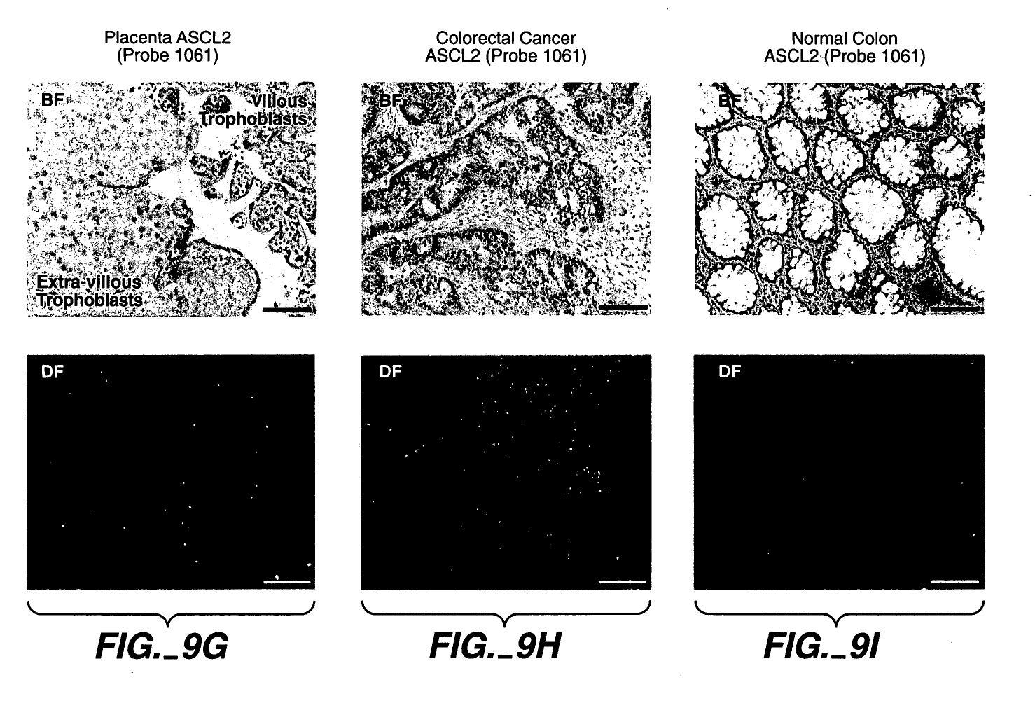

The 1071_P5/6 and 1061_P1/2 ASCL2 transcripts sense probes did not give a hybridization signal above background. Histological review of tissue sections hybridized with an anti-sense riboprobe (1071) against the unspliced ASCL2 transcript did not show any signal above background (FIG. 8). Of the normal tissues examined using the ASCL2 3′ probe 1061 (TMA H2001-688), only the extravillous trophoblasts of the human placenta showed significant hybridization (FIG. 9). However, a strong hybridization signal was observed in the neoplastic cells of a proportion of CRCs and cell lines (H2002-223, Dusseldorf TMAs) (FIG. 9). All tissue sections used showed a strong, positive signal when hybridized with the β-actin anti-sense probe (FIG. 9).

Quantitative phosphorimaging demonstrated a significant upregulation of ASCL2 in CRC vs. normal mucosa. The median expression level was increased four fold (154 vs. 39; p<0.0001) and eight fold (296 vs. 40; p<0.0001) respectively in the Dusseldorf TMAs and H2002-223. Of the colorectal cell pellets arrayed in H2002-223, expression was observed in SW620, COLO205, DLD1, HCC2998 and HCT15. HCT116, HT29 KM12 and SW480 pellets did not demonstrate expression above that seen in normal colon. The quantitative phosphorimaging score was significantly associated with the +/− score of hybridization to each core (data not shown, p<0.0001).

### Example 4

### Real-Time Polymerase Chain Reaction

4.1 Nucleic Acid Extraction

Total and poly(A)+ RNA were extracted from cell lines using RNA STAT60 (Tel-Test, Friendswood, Tex.) and FastTrack 2.0 (Invitrogen, Carlsbad, Calif.) respectively, according to the manufacturer's instructions. RNA was extracted from human tissues by CsCl precipitation. DNA was extracted from cell lines using DNA STAT60 (Tel-Test) according to the manufacturer's instructions. DNA was extracted from human tissues post CsCl RNA precipitation. Lysate supernatants were mixed with heavy phase lock gel (Brinkmann, Westbury, N.Y.) and 1.5 volumes of phenol:chloroform:isoamyl alcohol (25:24:1, pH 7.9). DNA was isolated in the aqueous phase by centrifugation at 2,600 rpm for 20 minutes. A second phenol:chloroform:isoamyl alcohol extraction was performed on the aqueous phase and the DNA precipitated in isopropanol, pelleted, washed in 80% ethanol and resuspended in TE buffer. Nucleic acid integrity and purity was checked by electrophoresis over a 1.2% agarose gel stained with ethidium bromide and spectrophotometry.

4.2 Validation

A master mix was prepared for each primer-probe set containing 1× buffer-A, 1.2 mM dNTPs (0.3 mM each of dATP, dCTP, dGTP, dTTP), 5 mM MgCl₂, 10 U of RNase Inhibitor, 25 U/ml Amplitaq-gold, 0.25 U/μl reverse transcriptase (Applied Biosystems, Foster City, Calif.), 0.33 ng/μl of each primer and 0.2 ng/μl of probe (reporter 5′FAM, quencher TAMRA-3′) (Table 8). Each 50 μl reaction was prepared in an optical PCR tube using 100 ng of total RNA (treated with DNase free, Ambion, Austin Tex.), 10 ng of Poly(A)+ RNA or 100 ng of genomic DNA. Thermal cycling (program 2) (Table 7) and quantitative analysis were carried out using an ABI Prism 7700 running Sequence Detection System v. 1.9 (Applied Biosystems).

The relative efficiency of each primer-probe set was characterized according to the criteria laid out by the manufacturer [ABI prism 7700 sequence detection system. In: User bulletin #2: Perkin-Elmer Corporation; 1997. P. 1-36]. The specificity of RT-PCR products was assessed by electrophoresis on a 4% agarose gel. All samples were represented in triplicate, in addition to no template and no reverse transcriptase control reactions. The mean Ct value from the three reactions was used for further analysis.

4.3 Application

The Hs.Scute_f/r/pl and RPL19 primer-probe sets were used to examine the expression of ASCL2 in total RNA from 25 CRCs, 16 normal tissues and 14 cell-lines. Similarly Hs.Scute_f/r/pl and RPL19 were used to amplify from a DNA template in a gene dosage analysis of ASCL2 copy number (relative to normal human genomic DNA) in five CRCs and seven cell-lines. To determine whether the spliced or unspliced transcript was responsible for the expression profile seen, primer-probe sets were designed to amplify from different regions of the ASCL2 message (FIG. 5)(Table 8). The experiment was performed on total RNA from four cell lines (HCT15, COLO205, JEG3 and HCT116) and normalized to RPL19.

4.4 Results

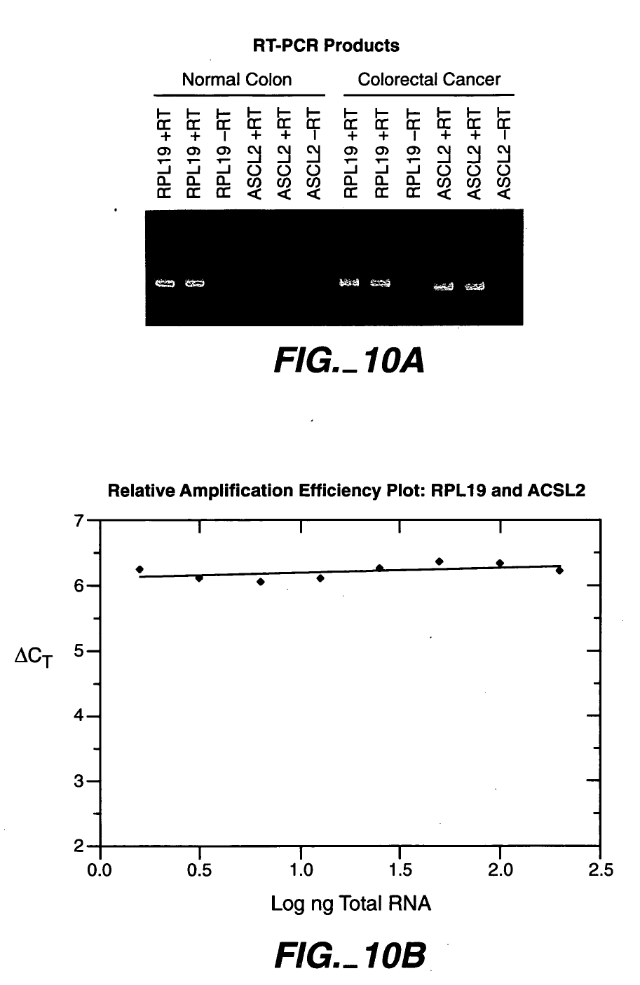

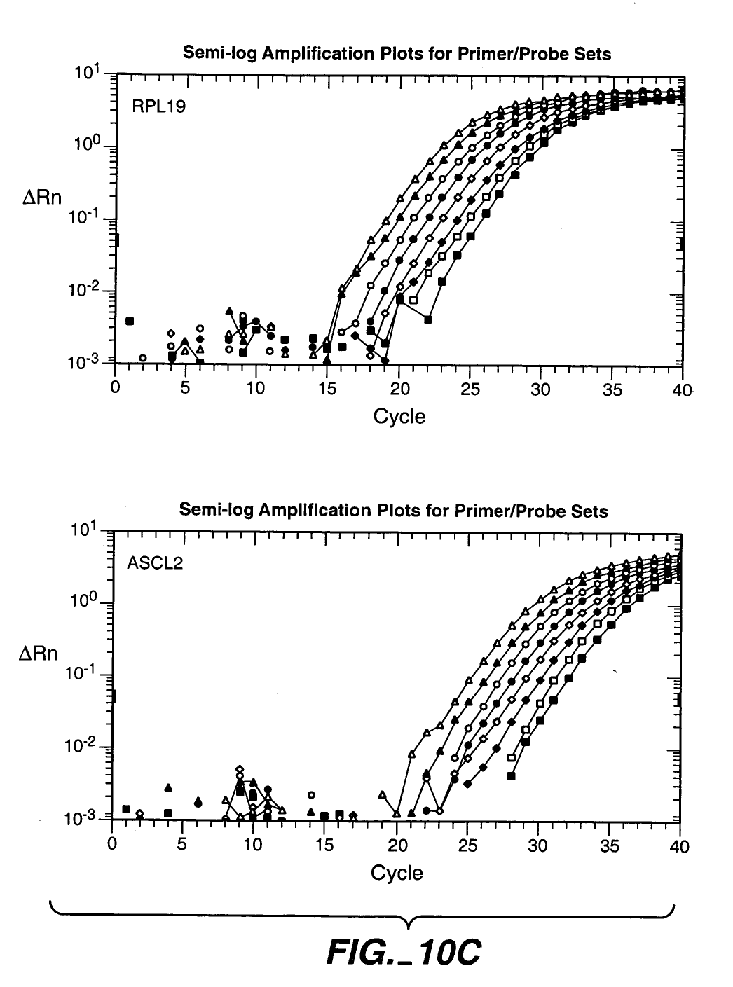

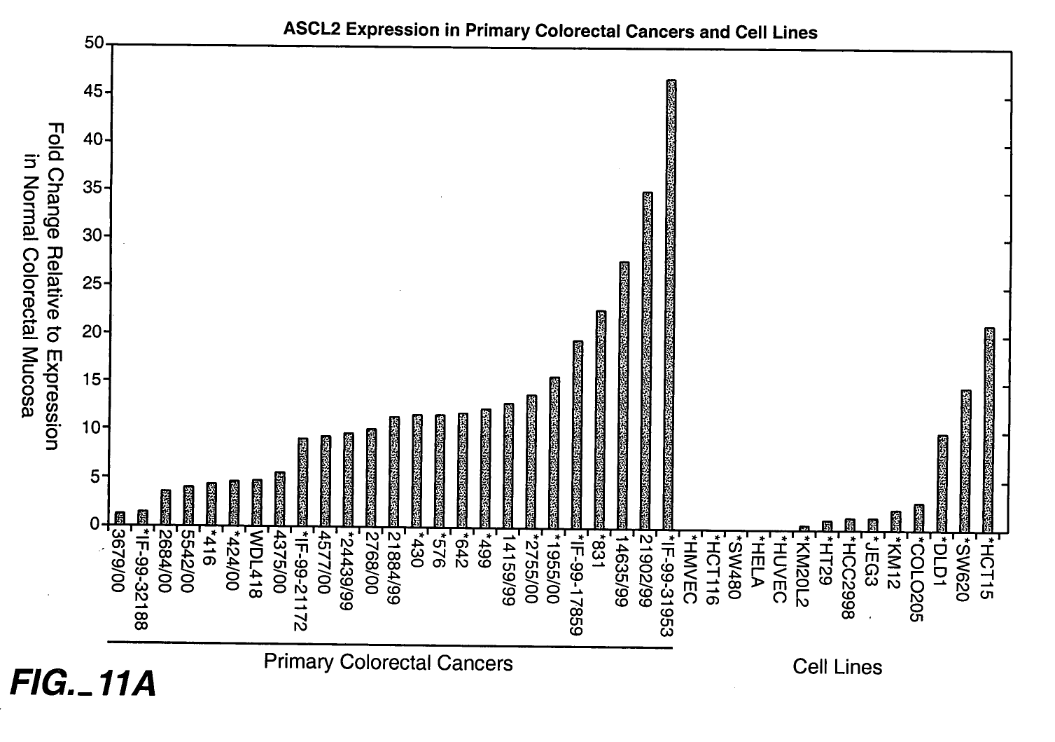

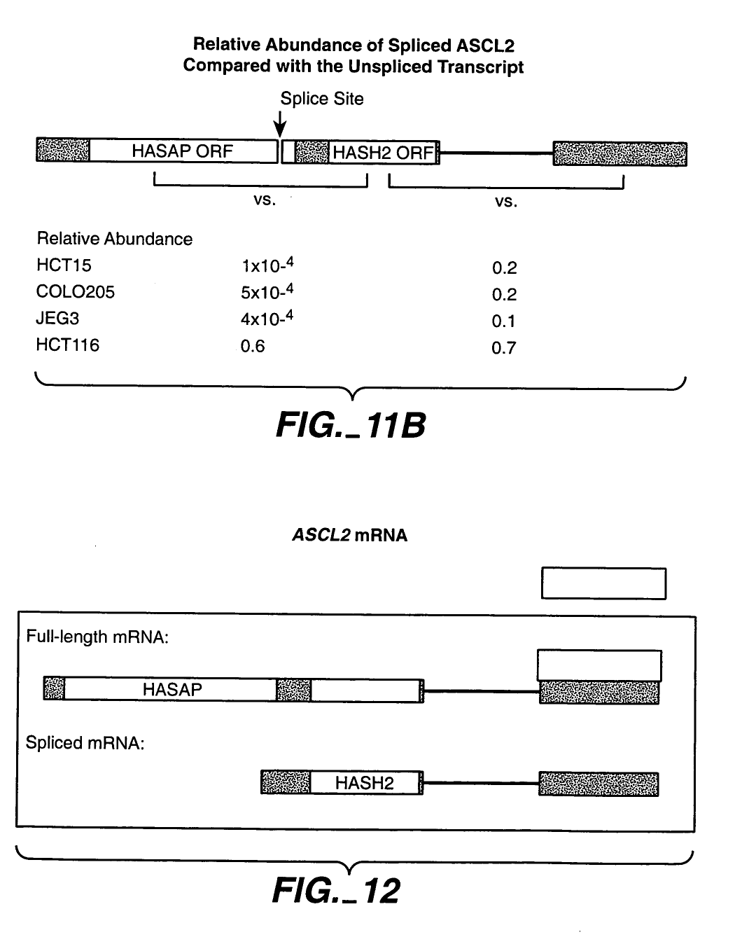

The results of these studies follows: All primer-probe sets were validated for the accurate quantitation of mRNA (FIG. 10). Median ASCL2 expression was 9.9 fold higher in CRC, compared with normal mucosa (95% confidence intervals 4.7-12.2) [FIG. 11a]. Sixty four percent of cases (n=18/28) showed upregulation >5 fold. This pattern was mirrored in the CRC cell lines (FIG. 11a). HU-U133 oligonucleotide microarray data was available for seven of the CRC RNA samples. There was a positive correlation in fold change when normalized to RPL19 expression in the corresponding normal mucosa (data not shown, R²=0.66 (n=7)].

The levels of ASCL2 template in the RNA samples decreased incrementally the more 5′ the probe. However, in cell lines that express ASCL2 at moderate to high levels, there is a much larger reduction in template amount 5′ to the splice site. This is not the case for cell lines, such as HCT116, which did not express high levels of ASCL2 (FIG. 11b).

### Example 5

### Cloning by Polymerase Chain Reaction

5.1 Plasmid Construction and Screening

The ASCL2 gene (designated as AF442769 in GenBank) is shown in FIG. 5. The full-length (unspliced) transcript contains two exons with two open reading frames (ORF) [designated HASAP ORF and HASH2 ORF respectively], the first open reading frame within the first exon is identified as encoding a polypeptide designated as HASAP (synonymous with TAT376); the second open reading frame within the first exon is identified as encoding a polypeptide designated as HASH2 (synonymous with TAT377). The corresponding spliced ASCL2 transcript corresponds to a splice within the first exon of the full-length unspliced gene which encodes the polypeptide identified herein as HASH2 (synonymous with TAT377). The corresponding ASCL2 mRNA transcript is shown in FIG. 12. In the present example, the AF442769 sequence was first confirmed against the corresponding murine genomic sequence in the Genbank (NT_{—}033238) and Celera Genomics (Rockville, Md.) databases and Incyte EST sequences with proprietary software written and developed at Genentech, Inc. cDNA probe templates were generated by reverse transcription of HCT-15 RNA using the Prostar kit (Stratagene, La Jolla, Calif.) according to the manufacturer's instructions. CRC and placental marathon-ready cDNA libraries were also screened. PCR reactions were prepared as described in EXAMPLE 4. The HASH2 (DNA327308) and HASAP (DNA327307) ORFs were amplified using HASH2_N_F/R1 primers in a two-round touchdown PCR (program 3) (FIG. 5) [Tables 6 and 7].

The HASAP ORF was similarly amplified using external and nested primers, respectively HASAP_N_F/R3 and HASAP_N_{F/R}3. Restriction sites (5′-XhoI and HindIII-3′) were introduced by amplifying with 327308. XhoI/HindIII and 327307. XhoI/HindIII primers respectively, using program 4 (Table 6). PCR products were purified using QlAquick (Qiagen) according to the manufacturer's instructions. The purified ORFs and the pEGFP-N1 vector (BD Clontech) were restricted for 2 hours at 37° C. with 60 U each of XhoI and HindIII (New England Biolabs (NEB), Beverly, Mass.). Restricted pEGFP-N1 was treated with calf-intestinal phosphatase (NEB) according to the manufacturer's instructions. The ORFs were gel-purified from a 1.2% agarose gel using the QIAquick kit. Ligation was performed overnight at 16° C. with T4 DNA ligase (NEB) according to the supplied protocol (restricted-vector only controls were included). Aliquots of DH5α-ft competent Escherichia coli (Invitrogen) were transformed with the ligation mixtures by heat-shock at 42° C. for 45 seconds. The E. coli were allowed to recover in SOC media at 37° C. for 1 hour, before being plated onto LB Kana (liquid broth with 50 μg/ml kanamycin) agar and incubated at 37° C. for 18 hours. Colonies were selected and grown in LB Kana over 18 hours for miniprep (Qiagen). Purified plasmids were screened for inserts by restriction with PstI (HASH2-pEGFP-N1) or SmaI (HASAP-pEGFP-N1) (NEB) according to manufacturer's instructions. Positive clones were sent for automated sequencing using vector- and insert-specific primers (Table 8). Sequence chromatograms were compared to the reference insert and vector sequences (GenBank AF442769, U55762) using Sequencher v.4.1 (Gene Codes, Ann Arbor, Mich.).

5.2 Transfection of Mammalian Cells

HCT116 cells were transfected using Fugene 6 (Roche Molecular Biochemicals, Indianapolis, Ind.) according to the manufacturer's instructions. Stable lines were made by growth in medium supplemented with 1000 μg/lm G418.

5.3 Results

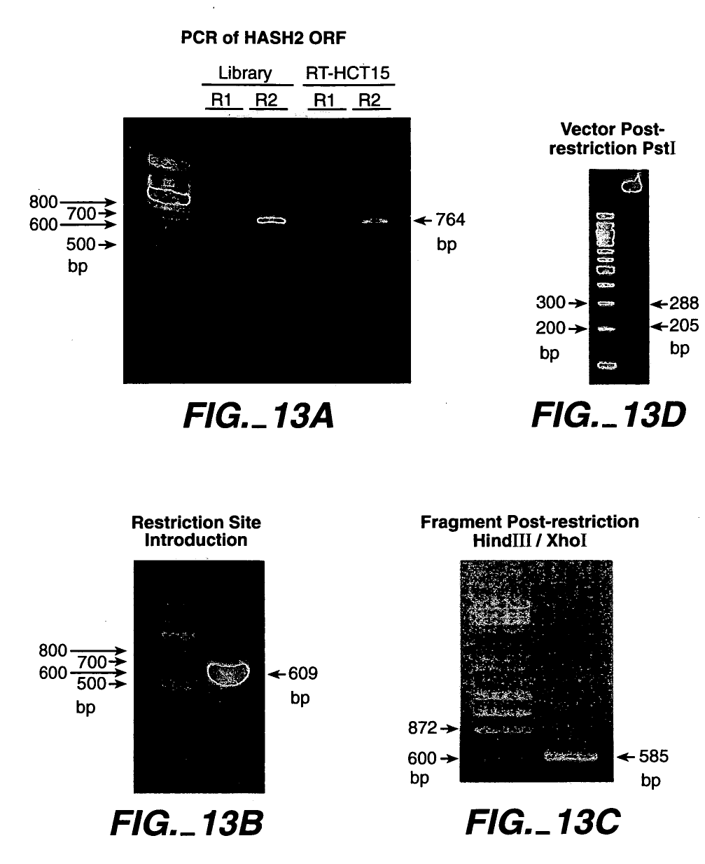

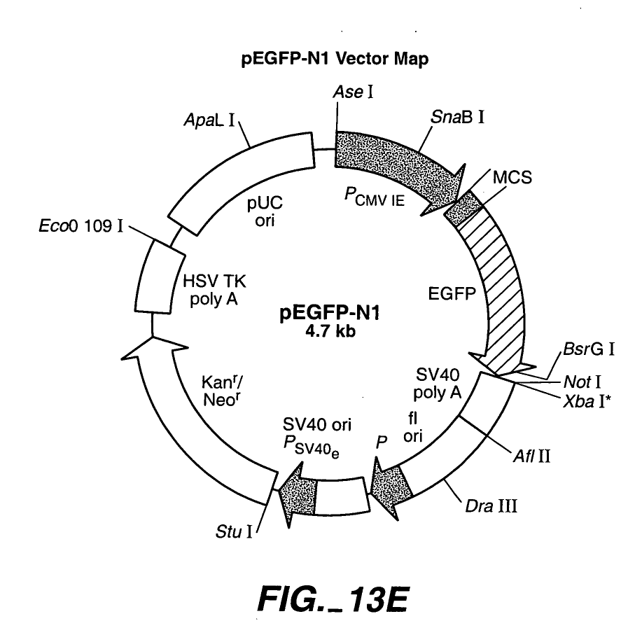

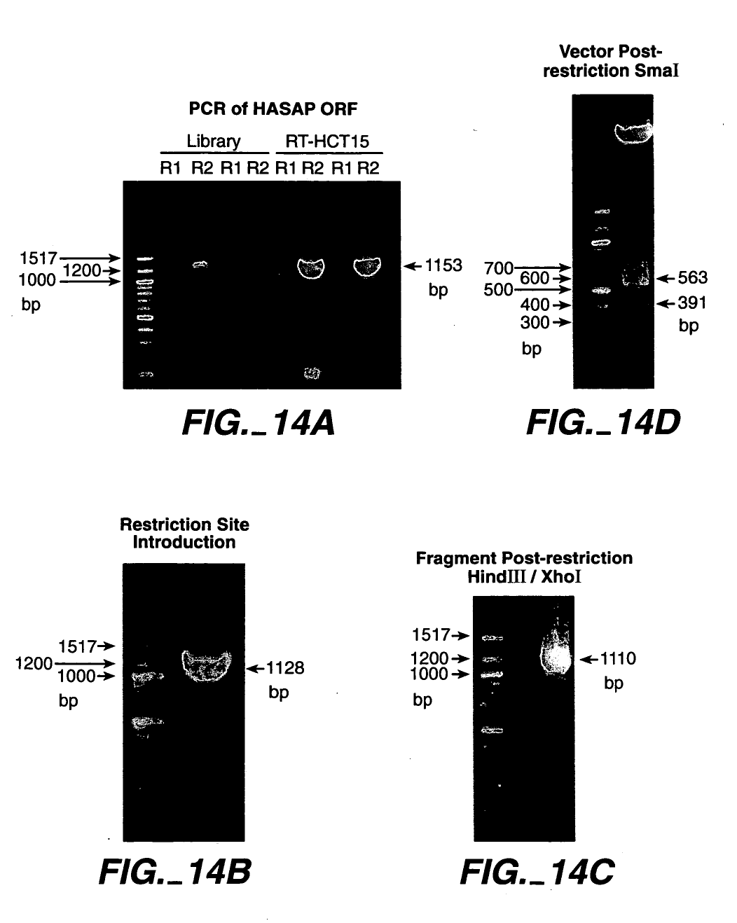

In summary, HASAP and HASH2 open reading frames were successfully cloned into the pEGFP-N1 vector (FIGS. 13 and 14). The sequences were wild-type to AF442769. The HASH2-pEGFP-N1 vector was transiently transfected into HCT116 cells and showed nuclear localization by fluorescent microscopy.

### Example 6

### Hybridization Studies

6.1 Synthesis and Labeling of the Probe

The HASAP_N_F/R3 and HASH2-N-F/R1 PCR products were gel purified for use as DNA probes (FIG. 5) (Table 6). Probes were labeled with [α³²P]-dCTP (ICN Pharmaceuticals, Costa Mesa, Calif.) using random hexamers (Rediprime II, Amersham Biosciences) according to the manufacturer's instructions. Unincorporated nucleotides were removed with a G₅₀-sephadex spin-column (Amersham Biosciences) and the efficiency of labeling was measured by a scintillation counter. To confirm probe labeling and purification, 100 ng was run over a 2% agarose gel, exposed to X-OMAT-AR film (Eastman Kodak).

6.2 Northern Blotting

6.2.1 Preparation

Two micrograms of HCT15, DLD1, JEG3 and HCT116 poly(A)+ RNA were each prepared in formaldehyde gel running buffer (50 mM sodium acetate, 0.2 M MOPS, 10 mM EDTA), 2.2 M formaldehyde and 50% formamide. Samples were incubated at 65° C. for 15 min and then cooled on ice. RNA was run in duplicate on a denaturing 1% agarose gel with 2.2 M formaldehyde and 1× formaldehyde gel running buffer at 4 V/cm. One half of the gel was cut and stained with ethidium bromide. Migration of ribosomal RNA bands 18 s and 28 s was measured under ultraviolet trans-illumination. The remaining gel piece was blotted onto Hybond-N nitrocellulose filters (Amersham Biosciences) over 6 hours. (Transfer efficiency was assessed by ethidium bromide staining of the blotted gel). The membrane was baked for 2 hours at 80° C. in a vacuum oven. Commercially available membranes (BD Clontech) representing a panel of normal tissue RNA were also included. Prehybridization was performed for 40 minutes at 68° C. in Quick-Hyb solution (Stratagene). Denatured probe was added to the hybridization solution to a final concentration of 1.25×10⁶ cpm/ml with 100 μg/ml denatured sonicated-fish sperm DNA. The membrane was hybridized for 3 hours at 68° C., washed twice at low stringency (15 minutes at room temperature, 2×SSC, 0.1% SDS) and twice at high stringency (15 minutes at 60° C., 0.2×SSC, 0.1% SDS) before exposure for 1 hour to a phosphorscreen, imaged with a Storm 840 (Amersham Biosciences). Autoradiography was performed overnight at −80° C. using X-OMAT-AR film (Eastman Kodak). 6.2.2 Results

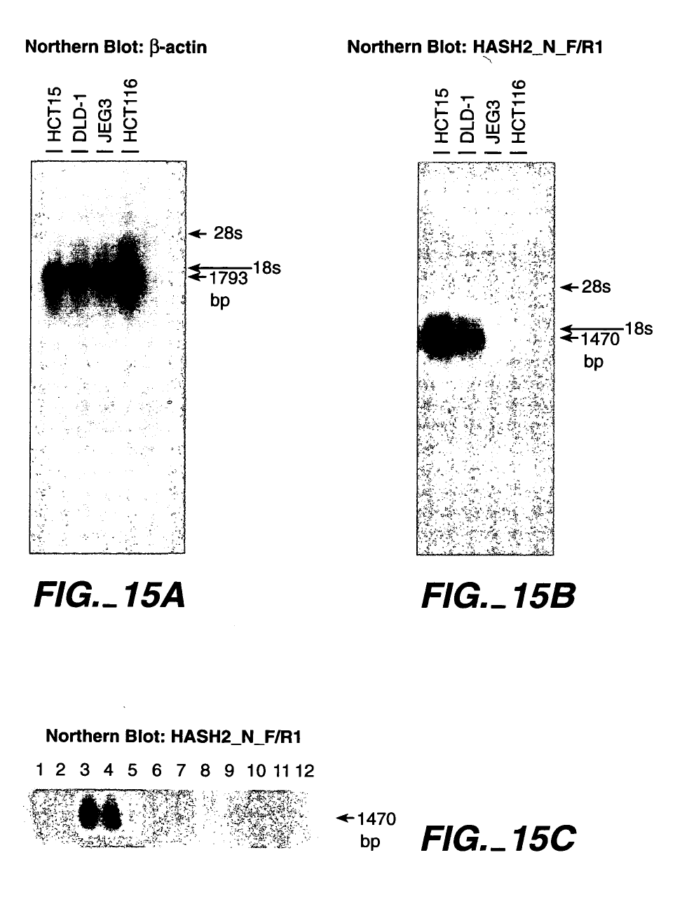

Equal loading and transfer of denaturing gels was confirmed by P-actin hybridization to all four lanes (FIG. 15a). The ASCL2 probe hybridized to produce a single band at 1470 bp, corresponding to the size of the spliced transcript (TAT377), in HCT15 and DLD1 (FIG. 15b). Hybridization to higher molecular weight bands was not observed. There was no appreciable hybridization to JEG3 or HCT116. Phosphorimage quantitation of the 1470 bp band significantly correlated with real-time RT-PCR data on the same RNA samples (data not shown; R²=0.94). Northern hybridization to a commercial blot of normal tissue RNA demonstrated hybridization to placenta and small intestine only (FIG. 15c).

6.3 cDNA Library Screen

6.3.1 Methods for cDNA Library Screening

In-house cDNA libraries were screened representing transcripts 0.2-2 kb and >2kb from normal human placenta (respectively LIB381 and 380), normal human colon (respectively LIB836 and 835) and the COLO205 cell line (respectively LIB688 and 687). All libraries were constructed from oligo(dT)-primed cDNA in the pRK5E vector. The libraries were first screened by PCR for the presence of the HASAP ORF as described above in EXAMPLE 5.

LIB687 showed the presence of the full-length ORF and was selected for further screening. Pooled E. coli transformed with this library (stored in 20% glycerol at −70° C.) were thawed into LB Carb (liquid broth with 50 μg/ml carbenicillin). Eight ten-fold dilutions were plated onto LB Carb agar and incubated overnight at 37° C. The density of the colonies was calculated and a dilution chosen to represent 6×10⁶ colonies, cultured overnight on LB Carb agar. Colonies were transferred to Hybond N+ nitrocellulose filters, denatured in 0.05 M NaOH, 0.15 M NaCl for 5 minutes, and neutralized 10 minutes in 1 M Tris, 1.5 M NaCl. Plates were allowed to recover for 8 hours at 37° C., before being stored at 4° C. The membrane was baked for 2 hours at 80° C. in a vacuum oven. Prehybridization was performed overnight at 42° C. in 0.1 ml/cm² hybridization solution (50% formamide, 5×SSC, 20 mM Tris-HCl (pH 7.6), 1× Denhardt's solution, 10% dextran sulfate and 0.1% SDS). Probe was denatured and added to the hybridization solution containing 100 μg/ml denatured, sonicated fish sperm DNA (Roche Molecular Biochemicals) to a final concentration of 1 ng/ml (>10⁹ dpm/μg). The membrane was hybridized overnight at 42° C., washed five times (15 minutes at 60° C., 0.2×SSC, 0.1% SDS) before being exposed overnight at −80° C. to X-OMAT-AR2 film. Positive colonies were cored from the corresponding plate and re-plated onto LB Carb agar until individual colonies could be selected and the vector purified by miniprep. Vectors were screened for inserts by XbaI digestion (NEB) and sequenced as described in EXAMPLE 4.

6.3.2 Results

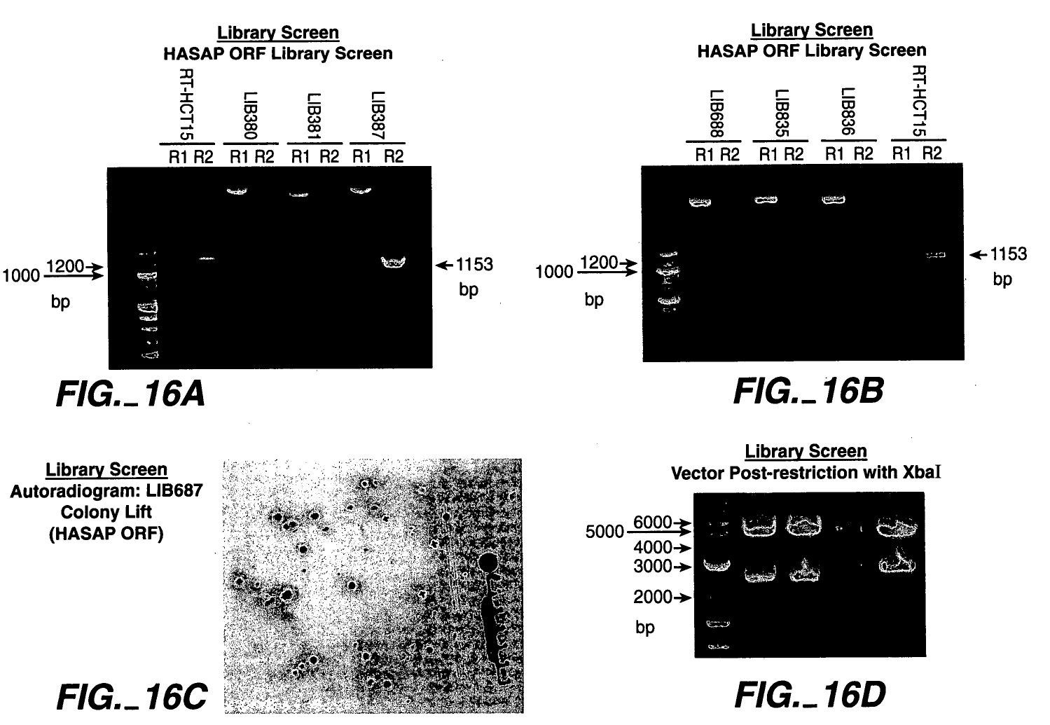

The results of the Library screening showed the abundance of the HASAP ORF in LIB688 was one (1) positive colony for every 1×10⁶ colonies plated (FIG. 16). However, the four clones sequenced were truncated towards the 3′ end of the ORF.

6.4 Validation at the Protein Level

6.4.1 Protein Extraction

Nuclear proteins were purified from mammalian cells using NE-PER nuclear and cytoplasmic extraction reagents (with 1:10 HALT protease-inhibitor cocktail) and quantified with the BCA protein assay (Pierce Biotechnology, Rockfor, Ill.) using a Spectra Max plate-reader (Molecular Devices, Sunnyvale, Calif.).

6.4.2 Polyclonal Antibody Production

Polyclonal antibodies were raised against synthetic peptides. The HASH2 58B antibody was raised against peptide sequence CGRASSSPGRGGSSEPGS (SEQ ID NO:8), HASAP 37A and 37 B antibodies were raised against peptide sequence CAHDWLRPWPPPPRPQEG (SEQ ID NO:9).

6.4.3 Flow Cytometry

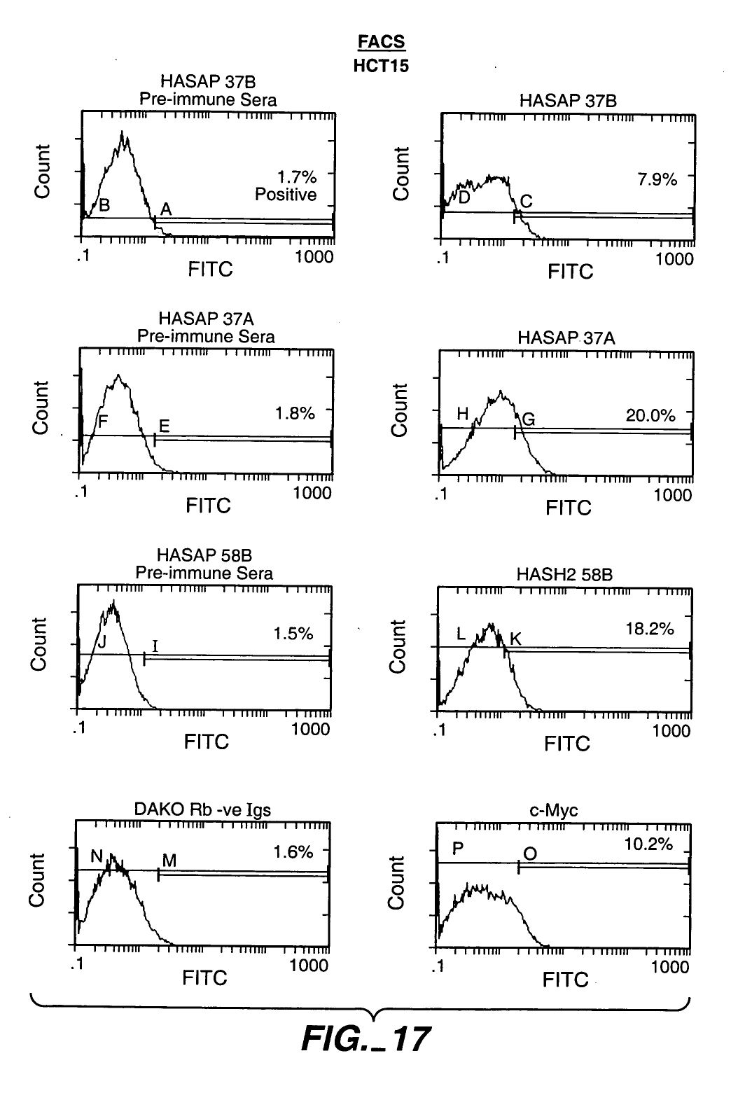

HCT15, HCT116 and JEG3 cells were fixed in 4% paraformaldehyde (in phosphate buffered saline (PBS)) on ice for 10 minutes and permeabilized at room temperature for 5 minutes in saponin buffer (0.1% saponin, 0.01% NaAzide, 1% fetal bovine serum in phosphate buffered saline, pH 7.2). Aliquots of 2×10⁶ cells treated with 0.1 μg/ml of goat anti-rabbit FITC-conjugated immunoglobulins (Caltag, Burlingame, Calif.). Appropriate negative control serum was included for each antibody in each cell line. The fluorescence intensity was measured on an Elite flow cytometer (Beckman Coulter). The results of these studies demonstrated that anti-c-Myc antibody labeled a larger proportion of cells in all samples than was observed with non-immunized rabbit immunoglobulins (FIG. 17). However, the HASAP and HASH2 antibodies only labeled HCT15 cells above the signal seen with pre-immune sera (see Table 9 below).

TABLE 9
Percentage of positive cells above the flow cytometry threshold
when labeled with antibody (+) or pre-immune serum(−)

| | c-Myc Antibody | | HASH2 58B Antibody | | HASAP 37A Antibody | | HASAP 37B Antibody | |
|---|---|---|---|---|---|---|---|---|
| Cell Line | + | − | + | − | + | − | + | − |
| HCT15 | 10.2 | 1.6 | 18.2 | 1.5 | 20.0 | 1.8 | 7.9 | 1.7 |
| JEG3 | 2.4 | 1.1 | 0.1 | 0.8 | 0.1 | 0.8 | 0.3 | 1.7 |
| HCT1165.0 | 1.5 | 0.1 | 0.8 | 0.1 | 0.7 | 0.1 | 0.7 | |

6.4.4 Western Blotting

Western blotting studies were also performed. Polyacrylamide gel electrophoresis was performed on 20 μg of denatured protein under reducing (1:10 NuPAGE sample reducing agent, Invitrogen) and non-reducing conditions. Ten percent bis-tris gels were run in MOPS SDS buffer with 1:10 anti-oxidant solution using a X-cell surelock minicell. Transfer to nitrocellulose membranes (0.2 μm pore diameter) was performed using the X-cell II blot module, all according to manufacturer's instructions (Invitrogen). (Identical gels were stained with Coomassie blue and protein transfer to the membranes was assessed by Ponceau-S staining). Membranes were blocked in 5% skimmed milk in tris-buffered saline with 0.1% tween-20 (pH 7.6) for 60 minutes at room temperature, before being incubated with the primary antibody (in 1% milk) for 60 minutes at room temperature. Primary antibodies included c-myc (rabbit polyclonal 2.0 μm/ml) (Novus Biologicals, Littleton, Colo.), HASH2 58B (2.9 μg/ml), HASAP 37A (1.6 μg/ml) and HASAP 37B (1.2 μg/ml). The rabbit antibodies were then labeled with goat immunoglobulins conjugated to horseradish peroxidase (DAKO Cytomation, Carpenteria, Calif.) 0.3 μg/ml in 1% milk for 60 minutes at room temperature. Immunocomplexes were visualized with extra-chemiluminescence and exposed to hyperfilm for 10 minutes (Amersham Bioscience).

6.4.5 Results of Western Blotting

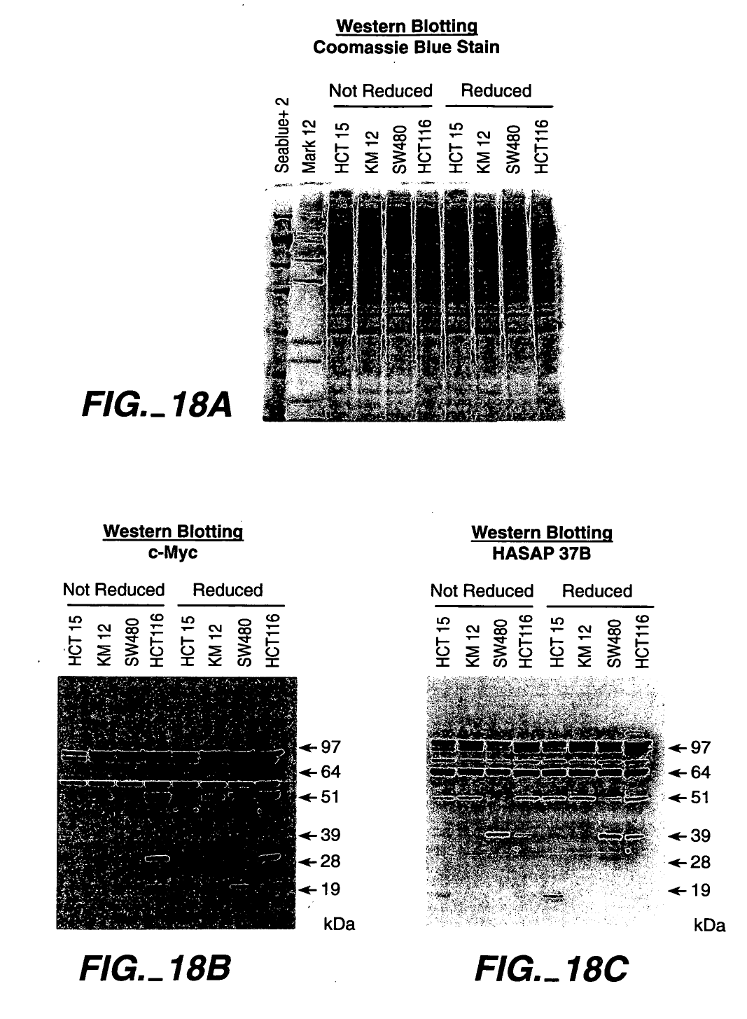

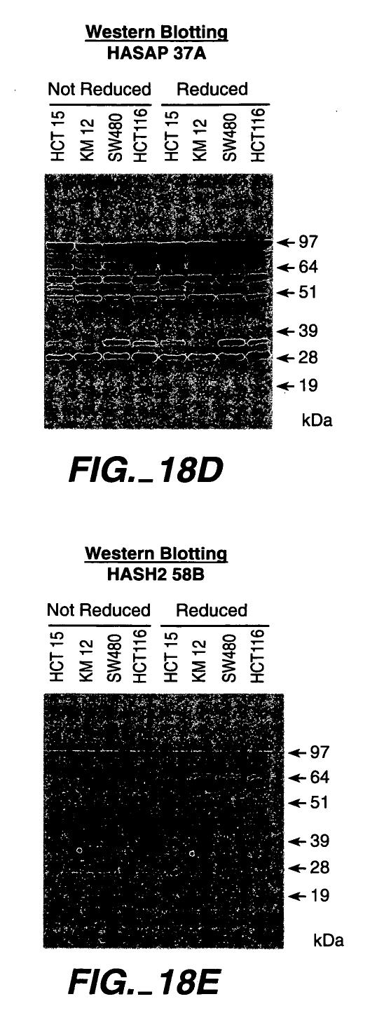

Coomassie blue staining of the gels indicated that the nuclear protein extracts were intact and loaded equally (FIG. 18a). All polyclonal antibodies gave multiple bands in the samples examined (FIGS. 18 b-d). Anti-c-myc showed immunoreactivity in HCT116 and SW480 protein lysates at the expected size of 27 kDa. The anti-HASAP antibodies both reacted with proteins at the expected size (39 kDa) in some of the lysates. However, there was no gradient in protein expression to reflect the expression levels observed at the mRNA level.

### Post-Analysis and Validation of Microarray Experiments

These studies have demonstrated a cancer-specific expression profile of ASCL2 in colorectal neoplasms. Gene Logic Affymetrix® DNA microarray studies showed an upregulation of ASCL2 in pre-malignant and malignant lesions of the colorectal mucosa. This was consistent across a large number of biological replicates applied to commercial microarrays. Although technical replicates were not performed, Affymetrix® asserts that only 1% of probesets will randomly show a 2 fold difference on replicate HG-U133 GeneChips. Upregulation was confirmed in silico by screening SAGE and EST libraries (EXAMPLE 2). In situ hybridization, real-time RT-PCR and Northern blotting data further corraborated the in silico findings (EXAMPLES 3-6) and strongly suggested that the HASAP ORF was not expressed at appreciable levels in CRC. The absence of an equivalent HASAP ORF in the mouse, the lack of any known domains or protein homology, and a low abundance of HASAP in the library screen indicates that HASAP may represent a genomic contaminant. Thus, the HASH2 gene appears to be the critical molecule which is overexpressed in colorectal tumors.

The data presented herein have shown the association between ASCL2 (HASH2) expression and CRC to be highly significant, largely specific to neoplasms of the large bowel and consistent across a range of methodologies. Upregulation in benign neoplasms of the large bowel argues for temporal precedence and suggests that HASH2 is important from an early stage in colorectal tumorgenesis.

### Example 7

### Use of TAT376 or TAT377 as a Hybridization Probe

The following method describes use of a nucleotide sequence encoding TAT376 or TAT377 as a hybridization probe for, i.e., diagnosis of the presence of a tumor in a mammal.

DNA comprising the coding sequence of full-length or mature TAT376 or TAT377 as disclosed herein can also be employed as a probe to screen for homologous DNAs (such as those encoding naturally-occurring variants of TAT376 or TAT377) in human tissue cDNA libraries or human tissue genomic libraries.

Hybridization and washing of filters containing either library DNAs is performed under the following high stringency conditions. Hybridization of radiolabeled TAT376- or TAT377-derived probe to the filters is performed in a solution of 50% formamide; 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence TAT376 or TAT377 can then be identified using standard techniques known in the art.

### Example 8

### Expression of TAT376 or TAT377 in E. coli

This example illustrates preparation of an unglycosylated form of TAT376 or TAT377 by recombinant expression in E. coli.

The DNA sequence encoding TAT376 or TAT377 is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the TAT376 or TAT377 coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized TAT376 or TAT377 protein can then be purified using a metal chelating column under conditions that allow tight binding of the protein.

TAT376 or TAT377 may be expressed in E. coli in a poly-His tagged form, using the following procedure. The DNA encoding TAT376 or TAT377 is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate•2H2O, 1.07 g KCl, 5.36 g Difco yeast extract, 5.36 g Sheffield hycase SF in 500 mL water, as we as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded TAT376 or TAT377 polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

TAT polypeptides disclosed herein have been successfully expressed and purified using this technique(s).

### Example 9

### Expression of TAT376 or TAT377 in mammalian cells

This example illustrates preparation of a potentially glycosylated form of TAT376 or TAT377 by recombinant expression in mammalian cells.

The vector, pRK5 (see EP 307,247, published Mar. 15, 1989), is employed as the expression vector. Optionally, the TAT376 or TAT377 DNA is ligated into pRK5 with selected restriction enzymes to allow insertion of the TAT376 or TAT377 DNA using ligation methods such as described in Sambrook et al., supra. The resulting vector is called pRK5-TAT376 or pRK5-TAT377.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-TAT376 or pRK5-TAT377 DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml 35S-methionine. After a 12 hour incubation, the conditioned medium is collected, concentrated on a spin filter, and loaded onto a 15% SDS gel. The processed gel may be dried and exposed to film for a selected period of time to reveal the presence of TAT376 or TAT377 polypeptide. The cultures containing transfected cells may undergo further incubation (in serum free medium) and the medium is tested in selected bioassays.

In an alternative technique, TAT376 or TAT377 may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-TAT DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed TAT376 or TAT377 can then be concentrated and purified by any selected method, such as dialysis and/or column chromatography.

In another embodiment, TAT376 or TAT377 can be expressed in CHO cells. The pRK5-TAT376 or pRK5-TAT377 can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of TAT376 or TAT377 polypeptide, the culture medium may be replaced with serum free medium. Preferably, the cultures are incubated for about 6 days, and then the conditioned medium is harvested. The medium containing the expressed TAT376 or TAT377 can then be concentrated and purified by any selected method.

Epitope-tagged TAT376 or TAT377 may also be expressed in host CHO cells. The TAT376 or TAT377 may be subcloned out of the pRK5 vector. The subclone insert can undergo PCR to fuse in frame with a selected epitope tag such as a poly-his tag into a Baculovirus expression vector. The poly-his tagged TAT376 or TAT377 insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged TAT376 or TAT377 can then be concentrated and purified by any selected method, such as by Ni²⁺-chelate affinity chromatography.

TAT376 or TAT377 may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugeneo (Boehringer Mannheim). The cells are grown as described in Lucas et al., supra. Approximately 3×10⁷ cells are frozen in an ampule for further growth and production as described below.

The ampules containing the plasmid DNA are thawed by placement into water bath and mixed by vortexing. The contents are pipetted into a centrifuge tube containing 10 ntrLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 iL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3 L production spinner is seeded at 1.2×10⁶ cells/mL. On day 0, the cell number pH ie determined. On day 1, the spinner is sampled and sparging with filtered air is commenced. On day 2, the spinner is sampled, the temperature shifted to 33° C., and 30 mL of 500 g/L glucose and 0.6 mnL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

TAT polypeptides disclosed herein have been successfully expressed and purified using this technique(s).

### Example 10

### Expression of TAT376 or TAT377 in Yeast

The following method describes recombinant expression of TAT376 or TAT377 in yeast.

First, yeast expression vectors are constructed for intracellular production or secretion of TAT376 or TAT377 from the ADH2/GAPDH promoter. DNA encoding TAT376 or TAT377 and the promoter is inserted into suitable restriction enzyme sites in the selected plasmid to direct intracellular-expression of TAT376 or TAT377. For secretion, DNA encoding TAT376 or TAT377 can be cloned into the selected plasmid, together with DNA encoding the ADH2/GAPDH promoter, a native TAT376 or TAT377 signal peptide or other mammalian signal peptide, or, for example, a yeast alpha-factor or invertase secretory signal/leader sequence, and linker sequences (if needed) for expression of TAT376 or TAT377.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant TAT376 or TAT377 can subsequently be isolated and purified by removing the yeast cells from the fermentation medium by centrifugation and then concentrating the medium using selected cartridge filters. The concentrate containing TAT376 or TAT377 may further be purified using selected column chromatography resins.

TAT polypeptides disclosed herein have been successfully expressed and purified using this technique(s).

### Example 11

### Expression of TAT376 or TAT377 in Baculovirus-Infected Insect Cells

The following method describes recombinant expression of TAT376 or TAT377 in Baculovirus-infected insect cells.

The sequence coding for TAT376 or TAT377 is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding TAT376 or TAT377 or the desired portion of the coding sequence of TAT376 or TAT377 such as the sequence encoding an extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Labbratorv Manual, Oxford: Oxford University Press (1994).

Expressed poly-his tagged TAT376 or TAT377 can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged TAT376 or TAT377 are pooled and dialyzed against loading buffer.

Alternatively, purification of the IgG tagged (or Fc tagged) TAT376 or TAT377 can be performed using known chromatography techniques, including for instance, Protein A or protein G column chromatography.

TAT polypeptides disclosed herein have been successfully expressed and purified using this technique(s).

### Example 12

### Preparation of Antibodies that Bind TAT376 or TAT377

This example illustrates preparation of monoclonal antibodies which can specifically bind TAT376 or TAT377.

Techniques for producing the monoclonal antibodies are known in the art and are described, for instance, in Goding, supra. Immunogens that may be employed include purified TAT376 or TAT377, fusion proteins containing TAT376 or TAT377, and cells expressing recombinant TAT376 or TAT377 on the cell surface. Selection of the immunogen can be made by the skilled artisan without undue experimentation.

Mice, such as Balb/c, are immunized with the TAT376 or TAT377 immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-TAT376 or anti-TAT377 antibodies.

After a suitable antibody titer has been detected, the animals “positive” for antibodies can be injected with a final intravenous injection of TAT376 or TAT377. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, amininopterin, and thymidine) medium to inhibit proliferation of non-fused cells, myeloma hybrids, and spleen cell hybrids.

The hybridoma cells will be screened in an ELISA for reactivity against TAT376 or TAT377. Determination of “positive” hybridoma cells secreting the desired monoclonal antibodies against TAT376 or TAT377 is within the skill in the art.

The positive hybridoma cells can be injected intraperitoneally into syngeneic Balb/c mice to produce ascites containing the anti-TAT376 or anti-TAT377 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 13

### Purification of TAT376 or TAT377 polypeptides Using Specific Antibodies

Native or recombinant TAT376 or TAT377 polypeptides may be purified by a variety of standard techniques in the art of protein purification. For example, pro-TAT376 or pro-TAT377 polypeptide, mature TAT376 or TAT377 polypeptide, or pre-TAT376 or pre-TAT377 polypeptide is purified by immunoaffinity chromatography using antibodies specific for the TAT376 or TAT377 polypeptide of interest. In general, an immunoaffinity column is constructed by covalently coupling the anti-TAT376 or anti-TAT377 polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of TAT376 or TAT377 polypeptide by preparing a fraction from cells containing TAT376 or TAT377 polypeptide in a soluble form. This preparation is derived by solubilization of the whole cell or of a subcellular fraction obtained via differential centrifugation by the addition of detergent or by other methods well known in the art. Alternatively, soluble TAT376 or TAT377 polypeptide containing a signal sequence may be secreted in useful quantity into the medium in which the cells are grown.

A soluble TAT376 or TAT377 polypeptide-containing preparation is passed over the immunoaffinity column, and the column is washed under conditions that allow the preferential absorbance of TAT376 or TAT377 polypeptide (e.g., high ionic strength buffers in the presence of detergent). Then, the column is eluted under conditions that disrupt antibody/TAT376 or antibody/TAT377 polypeptide binding (e.g., a low pH buffer such as approximately pH 2-3, or a high concentration of a chaotrope such as urea or thiocyanate ion), and TAT376 or TAT377 polypeptide is collected.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

1-10. (canceled)

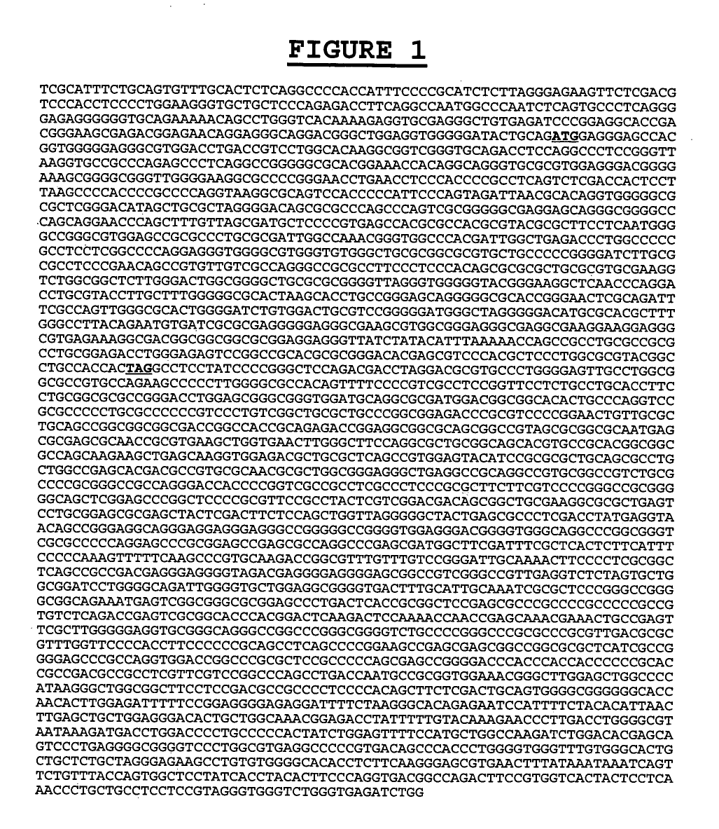

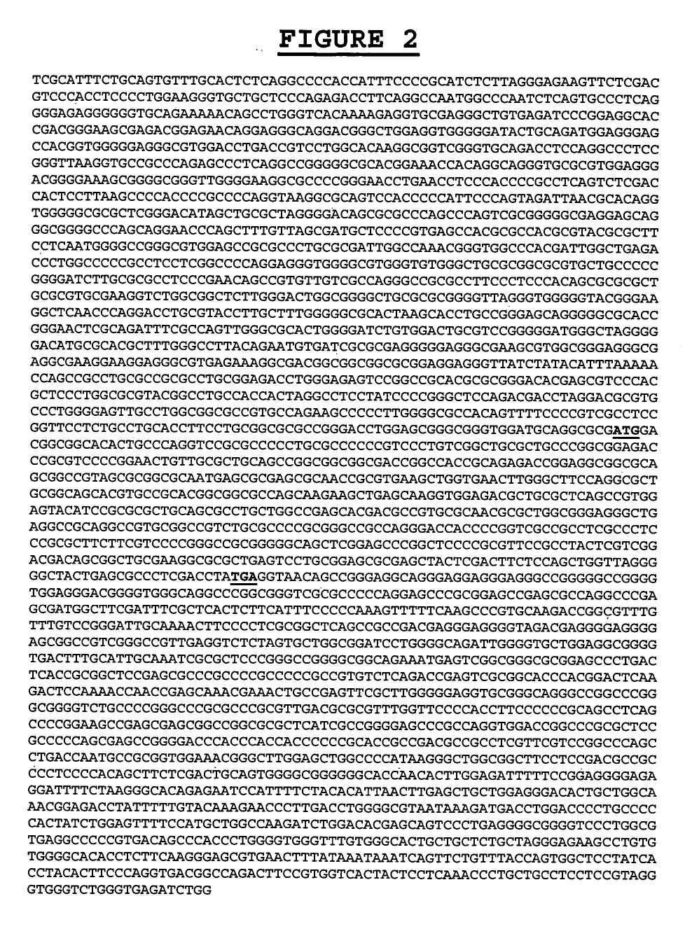

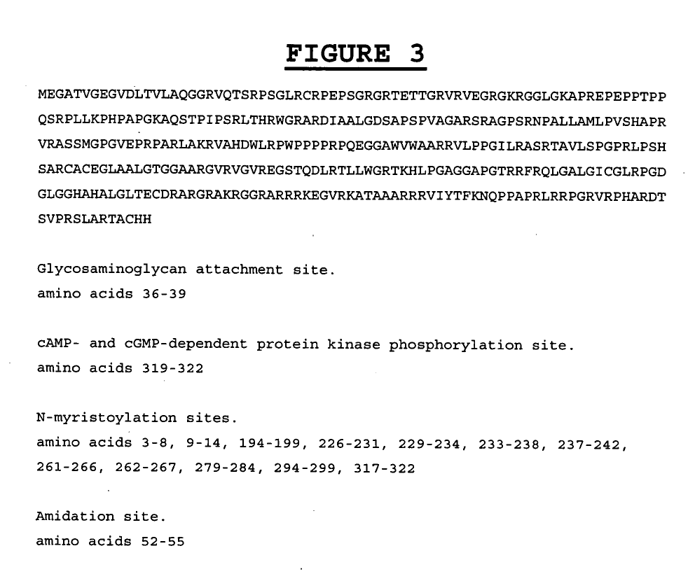

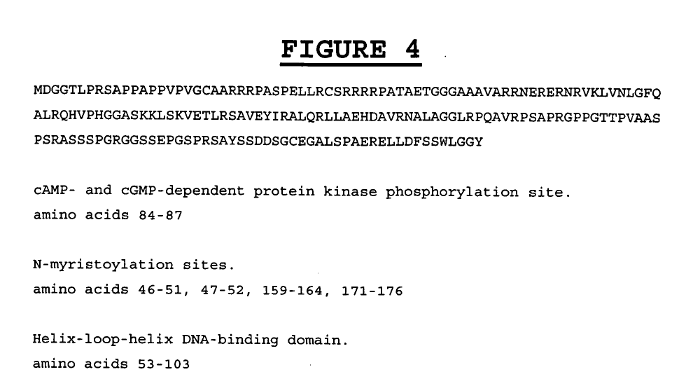

11. Isolated nucleic acid having a nucleotide sequence that has at least 95% nucleic acid sequence identity to:
(a) a DNA molecule encoding the amino acid sequence shown in any one of FIG. 3 or 4 (SEQ ID NOs:3 or 4);
(b) the nucleotide sequence shown in any one of FIG. 1 or 2 (SEQ ID NOs:1 or 2);
(c) the full-length coding region of the nucleotide sequence shown in any one of FIG. 1 or 2 (SEQ ID NOs:1 or 2); or (d) the complement of (a), (b) or (c).

12. Isolated nucleic acid having:
(a) a nucleotide sequence that encodes the amino acid sequence shown in any one of FIG. 3 or 4 (SEQ ID NOs:3 or 4);
(b) the nucleotide sequence shown in any one of FIG. 1 or 2 (SEQ ID NOs:1 or 2);
(c) the full-length coding region of the nucleotide sequence shown in any one of FIG. 1 or 2 (SEQ ID NOs:1 or 2); or
(d) the complement of (a), (b) or (c).

13. Isolated nucleic acid that hybridizes to:
(a) a nucleic acid that encodes the amino acid sequence shown in any one of FIG. 3 or 4 (SEQ ID NOs:3 or 4);
(b) the nucleotide sequence shown in any one of FIG. 1 or 2 (SEQ ID NOs:1 or 2);
(c) the full-length coding region of the nucleotide sequence shown in any one of FIG. 1 or 2 (SEQ ID NOs:1 or 2); or
(d) the complement of (a), (b) or (c).

14. The nucleic acid of claim 13, wherein the hybridization occurs under stringent conditions.

15. The nucleic acid of claim 13 which is at least about 5 nucleotides in length.

16. An expression vector comprising the nucleic acid of claim 11, 12 or 13.

17. The expression vector of claim 16, wherein said nucleic acid is operably linked to control sequences recognized by a host cell transformed with the vector.

18. A host cell comprising the expression vector of claim 17.

19. The host cell of claim 18 which is a CHO cell, an E. coli cell or a yeast cell.

20. A process for producing a polypeptide comprising culturing the host cell of claim 18 under conditions suitable for expression of said polypeptide and recovering said polypeptide from the cell culture.

21. An isolated polypeptide having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NOs:1 or 2); or
(c) a polypeptide encoded by the full-length coding region of the nucleotide sequence shown in any one of FIG. 1 or 2 (SEQ ID NOs:1 or 2).

22. An isolated polypeptide having:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

23. A chimeric polypeptide comprising the polypeptide of claim 21 or 22 fused to a heterologous polypeptide.

24. The chimeric polypeptide of claim 23, wherein said heterologous polypeptide is an epitope tag sequence or an Fc region of an immunoglobulin.

25. An isolated antibody that binds to a polypeptide having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NO:1 or 2); or
(c) a polypeptide encoded by the full-length coding region of the nucleotide sequence shown in any one of FIG. 1 or 2 (SEQ ID NOs:1 or 2).

26. An isolated antibody that binds to a polypeptide having:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

27. The antibody of claim 25 or 26 which is a monoclonal antibody.

28. The antibody of claim 25 or 26 which is an antibody fragment.

29. The antibody of claim 25 or 26 which is a chimeric or a humanized antibody.

30. The antibody of claim 25 or 26 which is conjugated to a growth inhibitory agent.

31. The antibody of claim 25 or 26 which is conjugated to a cytotoxic agent.

32. The antibody of claim 31, wherein the cytotoxic agent is selected from the group consisting of toxins, antibiotics, radioactive isotopes and nucleolytic enzymes.

33. The antibody of claim 31, wherein the cytotoxic agent is a toxin.

34. The antibody of claim 33, wherein the toxin is selected from the group consisting of maytansinoid and calicheamicin.

35. The antibody of claim 33, wherein the toxin is a maytansinoid.

36. The antibody of claim 25 or 26 which is produced in bacteria.

37. The antibody of claim 25 or 26 which is produced in CHO cells.

38. The antibody of claim 25 or 26 which induces death of a cell to which it binds.

39. The antibody of claim 25 or 26 which is detectably labeled.

40. An isolated nucleic acid having a nucleotide sequence that encodes the antibody of claim 25 or 26.

41. An expression vector comprising the nucleic acid of claim 40 operably linked to control sequences recognized by a host cell transformed with the vector.

42. A host cell comprising the expression vector of claim 41.

43. The host cell of claim 42 which is a CHO cell, an E. coli cell or a yeast cell.

44. A process for producing an antibody comprising culturing the host cell of claim 42 under conditions suitable for expression of said antibody and recovering said antibody from the cell culture.

45. An isolated oligopeptide that binds to a polypeptide having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NOs:1 or 2); or
(c) a polypeptide encoded by the full-length coding region of the nucleotide sequence shown in any one of FIG. 1 or 2 (SEQ ID NOs:1 or 2).

46. An isolated oligopeptide that binds to a polypeptide having:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

47. The oligopeptide of claim 35 or 36 which is conjugated to a growth inhibitory agent.

48. The oligopeptide of claim 35 or 36 which is conjugated to a cytotoxic agent.

49. The oligopeptide of claim 38, wherein the cytotoxic agent is selected from the group consisting of toxins, antibiotics, radioactive isotopes and nucleolytic enzymes.

50. The oligopeptide of claim 38, wherein the cytotoxic agent is a toxin.

51. The oligopeptide of claim 40, wherein the toxin is selected from the group consisting of maytansinoid and calicheamicin.

52. The oligopeptide of claim 40, wherein the toxin is a maytansinoid.

53. The oligopeptide of claim 35 or 36 which induces death of a cell to which it binds.

54. The oligopeptide of claim 35 or 36 which is detectably labeled.

55. A TAT376 or TAT377 binding organic molecule that binds to a polypeptide having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NO:1 or 2); or
(c) a polypeptide encoded by the full-length coding region of the nucleotide sequence shown in any one of FIG. 1 or 2 (SEQ ID NOs:1 or 2).

56. The organic molecule of claim 45 that binds to a polypeptide having:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

57. The organic molecule of claim 55 or 56 which is conjugated to a growth inhibitory agent.

58. The organic molecule of claim 55 or 56 which is conjugated to a cytotoxic agent.

59. The organic molecule of claim 58, wherein the cytotoxic agent is selected from the group consisting of toxins, antibiotics, radioactive isotopes and nucleolytic enzymes.

60. The organic molecule of claim 58, wherein the cytotoxic agent is a toxin.

61. The organic molecule of claim 60, wherein the toxin is selected from the group consisting of maytansinoid and calicheamicin.

62. The organic molecule of claim 60, wherein the toxin is a maytansinoid.

63. The organic molecule of claim 55 or 56 which induces death of a cell to which it binds.

64. The organic molecule of claim 55 or 56 which is detectably labeled.

65. A composition of matter comprising:
(a) the polypeptide of claim 21;
(b) the polypeptide of claim 22;
(c) the chimeric polypeptide of claim 23;
(d) the antibody of claim 25;
(e) the antibody of claim 26;
(f) the oligopeptide of claim 35;
(g) the oligopeptide of claim 36;
(h) the TAT376 or TAT377 binding organic molecule of claim 55; or
(i) the TAT376 or TAT377 binding organic molecule of claim 56; in combination with a carrier.

66. The composition of matter of claim 65, wherein said carrier is a pharmaceutically acceptable carrier.

67. An article of manufacture comprising:
(a) a container; and
(b) the composition of matter of claim 65 contained within said container.

68. The article of manufacture of claim 67 further comprising a label affixed to said container, or a package insert included with said container, referring to the use of said composition of matter for the therapeutic treatment of or the diagnostic detection of a cancer.

69. A method of inhibiting the growth of a cell that expresses a protein having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NOs:1 or 2); or
(c) a polypeptide encoded by the full-length coding region of the nucleotide sequence shown in any one of FIG. 1 or 2 (SEQ ID NOs:1 or 2), said method comprising contacting said cell with an antibody, oligopeptide or organic molecule that binds to said protein, the binding of said antibody, oligopeptide or organic molecule to said protein thereby causing an inhibition of growth of said cell.

70. The method of claim 69, wherein said antibody is a monoclonal antibody.

71. The method of claim 69, wherein said antibody is an antibody fragment.

72. The method of claim 69, wherein said antibody is a chimeric or a humanized antibody.

73. The method of claim 69, wherein said antibody, oligopeptide or organic molecule is conjugated to a growth inhibitory agent.

74. The method of claim 69, wherein said antibody, oligopeptide or organic molecule is conjugated to a cytotoxic agent.

75. The method of claim 74, wherein said cytotoxic agent is selected from the group consisting of toxins, antibiotics, radioactive isotopes and nucleolytic enzymes.

76. The method of claim 74, wherein the cytotoxic agent is a toxin.

77. The method of claim 76, wherein the toxin is selected from the group consisting of maytansinoid and calicheamicin.

78. The method of claim 76, wherein the toxin is a maytansinoid.

79. The method of claim 69, wherein said antibody is produced in bacteria.

80. The method of claim 69, wherein said antibody is produced in CHO cells.

81. The method of claim 69, wherein said cell is a cancer cell.

82. The method of claim 81, wherein said cancer cell is further exposed to radiation treatment or a chemotherapeutic agent.

83. The method of claim 81, wherein said cancer cell is selected from the group consisting of a breast cancer cell, a colorectal cancer cell, a lung cancer cell, an ovarian cancer cell, a central nervous system cancer cell, a liver cancer cell, a bladder cancer cell, a pancreatic cancer cell, a cervical cancer cell, a melanoma cell and a leukemia cell.

84. The method of claim 81, wherein said protein is more abundantly expressed by said cancer cell as compared to a normal cell of the same tissue origin.

85. The method of claim 69 which causes the death of said cell.

86. The method of claim 69, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

87. A method of therapeutically treating a mammal having a cancerous tumor comprising cells that express a protein having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NO:1 or 2); or
(c) a polypeptide encoded by the full-length coding region of the nucleotide sequence shown in any one of FIG. 1 or 2 (SEQ ID NOs:1 or 2), said method comprising administering to said mammal a therapeutically effective amount of an antibody, oligopeptide or organic molecule that binds to said protein, thereby effectively treating said mammal.

88. The method of claim 87, wherein said antibody is a monoclonal antibody.

89. The method of claim 87, wherein said antibody is an antibody fragment.

90. The method of claim 87, wherein said antibody is a chimeric or a humanized antibody.

91. The method of claim 87, wherein said antibody, oligopeptide or organic molecule is conjugated to a growth inhibitory agent.

92. The method of claim 87, wherein said antibody, oligopeptide or organic molecule is conjugated to a cytotoxic agent.

93. The method of claim 92, wherein said cytotoxic agent is selected from the group consisting of toxins, antibiotics, radioactive isotopes and nucleolytic enzymes.

94. The method of claim 92, wherein the cytotoxic agent is a toxin.

95. The method of claim 94, wherein the toxin is selected from the group consisting of maytansinoid and calicheamicin.

96. The method of claim 94, wherein the toxin is a maytansinoid.

97. The method of claim 87, wherein said antibody is produced in bacteria.

98. The method of claim 87, wherein said antibody is produced in CHO cells.

99. The method of claim 87, wherein said tumor is further exposed to radiation treatment or a chemotherapeutic agent.

100. The method of claim 87, wherein said tumor is a breast tumor, a colorectal tumor, a lung tumor, an ovarian tumor, a central nervous system tumor, a liver tumor, a bladder tumor, a pancreatic tumor, or a cervical tumor.

101. The method of claim 87, wherein said protein is more abundantly expressed by the cancerous cells of said tumor as compared to a normal cell of the same tissue origin.

102. The method of claim 87, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

103. A method of determining the presence of a protein in a sample suspected of containing said protein, wherein said protein has at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NOs:1 or 2); or
(c) a polypeptide encoded by the full-length coding region of the nucleotide sequence shown in any one of FIG. 1 or 2 (SEQ ID NOs:1 or 2), said method comprising exposing said sample to an antibody, oligopeptide or organic molecule that binds to said protein and determining binding of said antibody, oligopeptide or organic molecule to said protein in said sample, wherein binding of the antibody, oligopeptide or organic molecule to said protein is indicative of the presence of said protein in said sample.

104. The method of claim 103, wherein said sample comprises a cell suspected of expressing said protein.

105. The method of claim 104, wherein said cell is a cancer cell.

106. The method of claim 103, wherein said antibody, oligopeptide or organic molecule is detectably labeled.

107. The method of claim 105, wherein said cancer cell is selected from the group consisting of a breast cancer cell, a colorectal cancer cell, a lung cancer cell, an ovarian cancer cell, a central nervous system cancer cell, a liver cancer cell, a bladder cancer cell, a pancreatic cancer cell, a cervical cancer cell, a melanoma cell and a leukemia cell.

108. The method of claim 105, wherein said protein is more abundantly expressed by said cancer cell as compared to a normal cell of the same tissue origin.

109. The method of claim 103, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

110. A method of diagnosing the presence of a tumor in a mammal, said method comprising determining the level of expression of a gene encoding a protein having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NO:1 or 2); or
(c) a polypeptide encoded by the full-length coding region of the nucleotide sequence shown in any one of FIG. 1 or 2 (SEQ ID NOs:1 or 2), in a test sample of tissue cells obtained from said mammal and in a control sample of known normal cells of the same tissue origin, wherein a higher level of expression of said protein in the test sample, as compared to the control sample, is indicative of the presence of tumor in the mammal from which the test sample was obtained.

111. The method of claim 110, wherein the step of determining the level of expression of a gene encoding said protein comprises employing an oligonucleotide in an in situ hybridization or RT-PCR analysis.

112. The method of claim 110, wherein the step determining the level of expression of a gene encoding said protein comprises employing an antibody in an immunohistochemistry or Western blot analysis.

113. The method of claim 110, wherein said antibody, oligopeptide or organic molecule is detectably labeled.

114. The method of claim 110, wherein said test sample of tissue cells is obtained from an individual suspected of having a tumor.

115. The method of claim 114, wherein said tumor is breast tumor, a colorectal tumor, a lung tumor, an ovarian tumor, a central nervous system tumor, a liver tumor, a bladder tumor, a pancreatic tumor, a cervical tumor, a melanoma tumor or a leukemia tumor.

116. The method of claim 110, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

117. A method of diagnosing the presence of a tumor in a mammal, said method comprising contacting a test sample of tissue cells obtained from said mammal with an antibody, oligopeptide or organic molecule that binds to a protein having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NOs:1 or 2); or
(c) a polypeptide encoded by the full-length coding region of the nucleotide sequence shown in any one of FIG. 1 or 2 (SEQ ID NOs:1 or 2), and detecting the formation of a complex between said antibody, oligopeptide or organic molecule and said protein in the test sample, wherein the formation of a complex is indicative of the presence of a tumor in said mammal.

118. The method of claim 117, wherein said antibody, oligopeptide or organic molecule is detectably labeled.

119. The method of claim 117, wherein said test sample of tissue cells is obtained from an individual suspected of having a cancerous tumor.

120. The method of claim 117, wherein said tumor is breast tumor, a colorectal tumor, a lung tumor, an ovarian tumor, a central nervous system tumor, a liver tumor, a bladder tumor, a pancreatic tumor, a cervical tumor, a melanoma tumor or a leukemia tumor.

121. The method of claim 117, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:l or 2); or
(c) an amino acid sequence encoded by the full-length coding region of the nucleotide sequence shown in any one of FIG. 1 or 2 (SEQ ID NOs:1 or 2).

122. A method for treating or preventing a cell proliferative disorder associated with increased expression or activity of a protein having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NOs:1 or 2); or
(c) a polypeptide encoded by the full-length coding region of the nucleotide sequence shown in any one of FIG. 1 or 2 (SEQ ID NOs:1 or 2), said method comprising administering to a subject in need of such treatment an effective amount of an antagonist of said protein, thereby effectively treating or preventing said cell proliferative disorder.

123. The method of claim 122, wherein said cell proliferative disorder is cancer.

124. The method of claim 122, wherein said antagonist is an anti-TAT376 or anti-TAT377 polypeptide antibody,TAT376 or TAT377 binding oligopeptide,TAT376 or TAT377 binding organic molecule or antisense oligonucleotide.

125. The method of claim 122, wherein said antibody is a monoclonal antibody.

126. The method of claim 122, wherein said antibody is an antibody fragment.

127. The method of claim 122, wherein said antibody is a chimeric or a humanized antibody.

128. The method of claim 122, wherein said antibody, oligopeptide or organic molecule is conjugated to a growth inhibitory agent.

129. The method of claim 122, wherein said antibody, oligopeptide or organic molecule is conjugated to a cytotoxic agent.

130. The method of claim 129, wherein said cytotoxic agent is selected from the group consisting of toxins, antibiotics, radioactive isotopes and nucleolytic enzymes.

131. The method of claim 130, wherein said toxin is selected from the group consisting of maytansinoid and calicheamicin.

132. The method of claim 122, wherein said subject is further exposed to radiation treatment or a chemotherapeutic agent.

133. The method of claim 123, wherein said cancer is selected from the group consisting of breast cancer, colorectal cancer, lung cancer, ovarian cancer, central nervous system cancer, liver cancer, bladder cancer, pancreatic cancer, cervical cancer, melanoma cancer or leukemia.

134. The method of claim 122, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

135. A method of binding an antibody, oligopeptide or organic molecule to a cell that expresses a protein having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NOs:1 or 2); or
(c) a polypeptide encoded by the full-length coding region of the nucleotide sequence shown in any one of FIG. 1 or 2 (SEQ ID NOs:1 or 2), said method comprising contacting said cell with an antibody, oligopeptide or organic molecule that binds to said protein and allowing the binding of the antibody, oligopeptide or organic molecule to said protein to occur, thereby binding said antibody, oligopeptide or organic molecule to said cell.

136. The method of claim 135, wherein said antibody is a monoclonal antibody.

137. The method of claim 135, wherein said antibody is an antibody fragment.

138. The method of claim 135, wherein said antibody is a chimeric or a humanized antibody.

139. The method of claim 135, wherein said antibody, oligopeptide or organic molecule is conjugated to a growth inhibitory agent.

140. The method of claim 135, wherein said antibody, oligopeptide or organic molecule is conjugated to a cytotoxic agent.

141. The method of claim 140, wherein said cytotoxic agent is selected from the group consisting of toxins, antibiotics, radioactive isotopes and nucleolytic enzymes.

142. The method of claim 140, wherein the cytotoxic agent is a toxin.

143. The method of claim 142, wherein the toxin is selected from the group consisting of maytansinoid and calicheamicin.

144. The method of claim 143, wherein the toxin is a maytansinoid.

145. The method of claim 135, wherein said antibody is produced in bacteria.

146. The method of claim 135, wherein said antibody is produced in CHO cells.

147. The method of claim 135, wherein said cell is a cancer cell.

148. The method of claim 147, wherein said cancer cell is further exposed to radiation treatment or a chemotherapeutic agent.

149. The method of claim 147, wherein said cancer cell is selected from the group consisting of a breast cancer cell, a colorectal cancer cell, a lung cancer cell, an ovarian cancer cell, a central nervous system cancer cell, a liver cancer cell, a bladder cancer cell, a pancreatic cancer cell, a cervical cancer cell, a melanoma cell and a leukemia cell.

150. The method of claim 135, wherein said protein is more abundantly expressed by said cancer cell as compared to a normal cell of the same tissue origin.

151. The method of claim 135, which causes the death of said cell.

152. The method of claim 135, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

153. A method for inhibiting the growth of a cell, wherein the growth of said cell is at least in part dependent upon a growth potentiating effect of a protein having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NO:1 or 2); or
(c) a polypeptide encoded by the full-length coding region of the nucleotide sequence shown in any one of FIG. 1 or 2 (SEQ ID NOs:1 or 2), said method comprising contacting said protein with an antibody, oligopeptide or organic molecule that binds to said protein, there by inhibiting the growth of said cell.

154. The method of claim 153, wherein said cell is a cancer cell.

155. The method of claim 153, wherein said protein is expressed by said cell.

156. The method of claim 153, wherein the binding of said antibody, oligopeptide or organic molecule to said protein antagonizes a cell growth-potentiating activity of said protein.

157. The method of claim 153, wherein the binding of said antibody, oligopeptide or organic molecule to said protein induces the death of said cell.

158. The method of claim 153, wherein said antibody is a monoclonal antibody.

159. The method of claim 153, wherein said antibody is an antibody fragment.

160. The method of claim 153, wherein said antibody is a chimeric or a humanized antibody.

161. The method of claim 153, wherein said antibody, oligopeptide or organic molecule is conjugated to a growth inhibitory agent.

162. The method of claim 153, wherein said antibody, oligopeptide or organic molecule is conjugated to a cytotoxic agent.

163. The method of claim 162, wherein said cytotoxic agent is selected from the group consisting of toxins, antibiotics, radioactive isotopes and nucleolytic enzymes.

164. The method of claim 163, wherein the cytotoxic agent is a toxin.

165. The method of claim 164, wherein the toxin is selected from the group consisting of maytansinoid and calicheamicin.

166. The method of claim 164, wherein the toxin is a maytansinoid.

167. The method of claim 153, wherein said antibody is produced in bacteria.

168. The method of claim 153, wherein said antibody is produced in CHO cells.

169. The method of claim 154, wherein said cancer cell is selected from the group consisting of a breast cancer cell, a colorectal cancer cell, a lung cancer cell, an ovarian cancer cell, a central nervous system cancer cell, a liver cancer cell, a bladder cancer cell, a pancreatic cancer cell, a cervical cancer cell, a melanoma cell and a leukemia cell.

170. The method of claim 154, wherein said protein is more abundantly expressed by said cancer cell as compared to a normal cell of the same tissue origin.

171. The method of claim 153, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

172. A method of therapeutically treating a tumor in a mammal, wherein the growth of said tumor is at least in part dependent upon a growth potentiating effect of a protein having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NO:1 or 2); or
(c) a polypeptide encoded by the full-length coding region of the nucleotide sequence shown in any one of FIG. 1 or 2 (SEQ ID NOs:1 or 2), said method comprising contacting said protein with an antibody, oligopeptide or organic molecule that binds to said protein, thereby effectively treating said tumor.

173. The method of claim 171, wherein said protein is expressed by cells of said tumor.

174. The method of claim 171, wherein the binding of said antibody, oligopeptide or organic molecule to said protein antagonizes a cell growth-potentiating activity of said protein.

175. The method of claim 171, wherein said antibody is a monoclonal antibody.

176. The method of claim 171, wherein said antibody is an antibody fragment.

177. The method of claim 171, wherein said antibody is a chimeric or a humanized antibody.

178. The method of claim 171, wherein said antibody, oligopeptide or organic molecule is conjugated to a growth inhibitory agent.

179. The method of claim 171, wherein said antibody, oligopeptide or organic molecule is conjugated to a cytotoxic agent.

180. The method of claim 178, wherein said cytotoxic agent is selected from the group consisting of toxins, antibiotics, radioactive isotopes and nucleolytic enzymes.

181. The method of claim 178, wherein the cytotoxic agent is a toxin.

182. The method of claim 180, wherein the toxin is selected from the group consisting of maytansinoid and calicheamicin.

183. The method of claim 180, wherein the toxin is a maytansinoid.

184. The method of claim 171, wherein said antibody is produced in bacteria.

185. The method of claim 171, wherein said antibody is produced in CHO cells.

186. The method of claim 172, wherein said tumor is breast tumor, a colorectal tumor, a lung tumor, an ovarian tumor, a central nervous system tumor, a liver tumor, a bladder tumor, a pancreatic tumor, a cervical tumor, a melanoma tumor or a leukemia tumor.

187. The method of claim 172, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

188. A method of identifying a molecule which inhibits the activity of the polypeptide of SEQ ID Nos: 3 or 4, wherein said method comprises screening one or more molecules for a molecule that inhibits the activity of the polypeptide of SEQ ID Nos. 3 or 4.

189. The method of claim 188 wherein said method comprises contacting cells expressing the polypeptide of SEQ ID NO: 3 or 4 with a candidate molecule and detecting the inhibition of the activity of said polypeptide.

190. A method of claim 188 wherein said molecule binds to the basic domain or helix-loop-helix domain of the polypeptide of SEQ ID NO: 3 or 4.

191. The method of claim 188 wherein said molecule is a chemical compound.

192. The method for screening for the presence of a molecule that affects the interaction between the polypeptide of SEQ ID Nos. 3 or 4 and a second polypeptide or nucleic acid, comprising:
(a) contacting in a cell the molecule with the polypeptide of SEQ ID Nos. 3 or 4 wherein association of the polypeptide of SEQ ID Nos. 3 or 4 with a second polypeptide or nucleic acid in the presence of the molecule results in a detectable response by changing expression of a detectable gene or gene product; and
(b) comparing the detectable response in the presence of the molecule and the polypeptide of SEQ ID Nos. 3 or 4 and the second polypeptide or nucleic acid with the detectable response in the absence of the molecule, wherein a difference in response is indicative of the polypeptide of SEQ ID Nos. 3 or 4 interacting with a second polypeptide or nucleic acid and a molecule that affects said interaction.

193. The method of claim 192, where at least said polypeptide of SEQ ID Nos. 3 or 4 contains a basic DNA binding domain or a helix-loop-helix heterodimerization domain

194. The method of claim 192 wherein the detectable response is produced from a gene encoding a protein selected from the group consisting of β-galactosidase, green fluorescent protein, luciferase, alkaline phosphatase and chloramphenicol acetyl transferase.

195. The method of claim 194 wherein the detectable response is produced from a gene encoded by a gene expressed in the host cell.

196. The method of claim 195 wherein the host cell further comprises a first recombinant gene encoding the polypeptide of SEQ ID Nos. 3 or 4 and a second recombinant gene encoding the second polypeptide.

197. A method of detecting the presence of cells in a sample suspected of containing said cells, said method comprising detecting said cells by detecting cells expressing a polypeptide comprising an amino acid sequence having at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NO:1 or 2); or
(c) a polypeptide encoded by the full-length coding region of the nucleotide sequence shown in any one of FIG. 1 or 2 (SEQ ID NOs:1 or 2), said method comprising contacting said sample with an antibody, oligopeptide or organic molecule that binds to said protein and detecting the formation of a complex between said antibody, oligopeptide or organic molecule, wherein the formation of a complex indicates the presence of said cell.

198. The method of claim 197, wherein said antibody is a monoclonal antibody.

199. The method of claim 197, wherein said antibody is an antibody fragment.

200. The method of claim 197, wherein said antibody is a chimeric or a humanized antibody.

201. The method of claim 197, wherein said antibody, oligopeptide or organic molecule is detectably labeled.

202. The method of claim 197, wherein said test sample of tissue cells is obtained from an individual suspected of having a tumor.

203. The method of claim 202, wherein said tumor is breast tumor, a colorectal tumor, a lung tumor, an ovarian tumor, a central nervous system tumor, a liver tumor, a bladder tumor, a pancreatic tumor, a cervical tumor, a melanoma tumor or a leukemia tumor.

204. The method of claim 197, wherein said complex formation is detected in an increased number of cells in said sample, as compared to a control sample.

205. The method of claim 197, wherein said cell is a cancer cell.

206. The method of claim 205, wherein said cancer cell is selected from the group consisting of a breast cancer cell, a colorectal cancer cell, a lung cancer cell, an ovarian cancer cell, a central nervous system cancer cell, a liver cancer cell, a bladder cancer cell, a pancreatic cancer cell, a cervical cancer cell, a melanoma cell and a leukemia cell.

207. The method of claim 205, wherein said protein is more abundantly expressed by said cancer cell as compared to a normal cell of the same tissue origin.

208. The method of claim 197, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).

209. A method of determining the presence of a protein in a sample suspected of containing said protein, wherein said protein has at least 95% amino acid sequence identity to:
(a) the polypeptide shown in any one of FIG. 3 or 4 (SEQ ID NOs:3 or 4);
(b) a polypeptide encoded by the nucleotide sequence shown in any one of FIG. 1 or 2 (SEQ ID NOs:1 or 2); or
(c) a polypeptide encoded by the full-length coding region of the nucleotide sequence shown in any one of FIG. 1 or 2 (SEQ ID NOs:1 or 2), said method comprising exposing said sample to an antibody, oligopeptide or organic molecule that binds to said protein and determining binding of said antibody, oligopeptide or organic molecule to said protein in said sample, wherein binding of the antibody, oligopeptide or organic molecule to said protein is indicative of the presence of said protein in said sample.

210. The method of claim 209, wherein said sample comprises a cell suspected of expressing said protein.

211. The method of claim 209, wherein said antibody is a monoclonal antibody.

212. The method of claim 209, wherein said antibody is an antibody fragment.

213. The method of claim 209, wherein said antibody is a chimeric or a humanized antibody.

214. The method of claim 209, wherein said antibody, oligopeptide or organic molecule is detectably labeled.

215. The method of claim 209, wherein said cell is a cancer cell.

216. The method of claim 215, wherein said cancer cell is selected from the group consisting of a breast cancer cell, a colorectal cancer cell, a lung cancer cell, an ovarian cancer cell, a central nervous system cancer cell, a liver cancer cell, a bladder cancer cell, a pancreatic cancer cell, a cervical cancer cell, a melanoma cell and a leukemia cell.

217. The method of claim 215, wherein said protein is more abundantly expressed by said cancer cell as compared to a normal cell of the same tissue origin.

218. The method of claim 209, wherein said protein has:
(a) the amino acid sequence shown in any one of FIG. 3 or 4 (SEQ ID NOs:3 or 4);
(b) an amino acid sequence encoded by the nucleotide sequence shown in any one of FIG. 1 or 2 (SEQ ID NOs:1 or 2); or
(c) an amino acid sequence encoded by the full-length coding region of the nucleotide sequence shown in any one of FIG. 1 or 2 (SEQ ID NOs:1 or 2).


SEQUENCE LISTING (57 sequences)

SEQ ID NO: 1 - DNA, 3500 bp, Homo sapien
tcgcatttctgcagtgtttgcactctcaggccccaccatttccccgcatctcttagggagaagttctcgacgtcccacctcccctggaagggtgctgctcccagagaccttcaggccaatggcccaatctcagtgccctcaggggagaggggggtgcagaaaaacagcctgggtcacaaaagaggtgcgagggctgtgagatcccggaggcaccgacgggaagcgagacggagaacaggagggcaggacgggctggaggtgggggatactgcagatggagggagccacggtgggggagggcgtggacctgaccgtcctggcacaaggcggtcgggtgcagacctccaggccctccgggttaaggtgccgcccagagccctcaggccgggggcgcacggaaaccacaggcagggtgcgcgtggagggacggggaaagcggggcgggttggggaaggcgccccgggaacctgaacctcccaccccgcctcagtctcgaccactccttaagccccaccccgccccaggtaaggcgcagtccacccccattcccagtagattaacgcacaggtgggggcgcgctcgggacatagctgcgctaggggacagcgcgcccagcccagtcgcgggggcgaggagcagggcggggcccagcaggaacccagctttgttagcgatgctccccgtgagccacgcgccacgcgtacgcgcttcctcaatggggccgggcgtggagccgcgccctgcgcgattggccaaacgggtggcccacgattggctgagaccctggcccccgcctcctcggccccaggagggtggggcgtgggtgtgggctgcgcggcgcgtgctgcccccggggatcttgcgcgcctcccgaacagccgtgttgtcgccagggccgcgccttccctcccacagcgcgcgctgcgcgtgcgaaggtctggcggctcttgggactggcggggctgcgcgcggggttagggtgggggtacgggaaggctcaacccaggacctgcgtaccttgctttgggggcgcactaagcacctgccgggagcagggggcgcaccgggaactcgcagatttcgccagttgggcgcactggggatctgtggactgcgtccgggggatgggctagggggacatgcgcacgctttgggccttacagaatgtgatcgcgcgagggggagggcgaagcgtggcgggagggcgaggcgaaggaaggagggcgtgagaaaggcgacggcggcggcgcggaggagggttatctatacatttaaaaaccagccgcctgcgccgcgcctgcggagacctgggagagtccggccgcacgcgcgggacacgagcgtcccacgctccctggcgcgtacggcctgccaccactaggcctcctatccccgggctccagacgacctaggacgcgtgccctggggagttgcctggcggcgccgtgccagaagcccccttggggcgccacagttttccccgtcgcctccggttcctctgcctgcaccttcctgcggcgcgccgggacctggagcgggcgggtggatgcaggcgcgatggacggcggcacactgcccaggtccgcgccccctgcgccccccgtccctgtcggctgcgctgcccggcggagacccgcgtccccggaactgttgcgctgcagccggcggcggcgaccggccaccgcagagaccggaggcggcgcagcggccgtagcgcggcgcaatgagcgcgagcgcaaccgcgtgaagctggtgaacttgggcttccaggcgctgcggcagcacgtgccgcacggcggcgccagcaagaagctgagcaaggtggagacgctgcgctcagccgtggagtacatccgcgcgctgcagcgcctgctggccgagcacgacgccgtgcgcaacgcgctggcgggagggctgaggccgcaggccgtgcggccgtctgcgccccgcgggccgccagggaccaccccggtcgccgcctcgccctcccgcgcttcttcgtccccgggccgcgggggcagctcggagcccggctccccgcgttccgcctactcgtcggacgacagcggctgcgaaggcgcgctgagtcctgcggagcgcgagctactcgacttctccagctggttagggggctactgagcgccctcgacctatgaggtaacagccgggaggcagggaggagggagggccgggggccggggtggagggacggggtgggcaggcccggcgggtcgcgcccccaggagcccgcggagccgagcgccaggcccgagcgatggcttcgatttcgctcactcttcatttcccccaaagtttttcaagcccgtgcaagaccggcgtttgtttgtccgggattgcaaaacttcccctcgcggctcagccgccgacgagggaggggtagacgaggggaggggagcggccgtcgggccgttgaggtctctagtgctggcggatcctggggcagattggggtgctggaggcggggtgactttgcattgcaaatcgcgctcccgggccggggcggcagaaatgagtcggcgggcgcggagccctgactcaccgcggctccgagcgcccgccccgcccccgccgtgtctcagaccgagtcgcggcacccacggactcaagactccaaaaccaaccgagcaaacgaaactgccgagttcgcttgggggaggtgcgggcagggccggcccgggcggggtctgccccgggcccgcgcccgcgttgacgcgcgtttggttccccaccttccccccgcagcctcagccccggaagccgagcgagcggccggcgcgctcatcgccggggagcccgccaggtggaccggcccgcgctccgcccccagcgagccggggacccacccaccaccccccgcaccgccgacgccgcctcgttcgtccggcccagcctgaccaatgccgcggtggaaacgggcttggagctggccccataagggctggcggcttcctccgacgccgcccctccccacagcttctcgactgcagtggggcggggggcaccaacacttggagatttttccggaggggagaggattttctaagggcacagagaatccattttctacacattaacttgagctgctggagggacactgctggcaaacggagacctatttttgtacaaagaacccttgacctggggcgtaataaagatgacctggacccctgcccccactatctggagttttccatgctggccaagatctggacacgagcagtccctgaggggcggggtccctggcgtgaggcccccgtgacagcccaccctggggtgggtttgtgggcactgctgctctgctagggagaagcctgtgtggggcacacctcttcaagggagcgtgaactttataaataaatcagttctgtttaccagtggctcctatcacctacacttcccaggtgacggccagacttccgtggtcactactcctcaaaccctgctgcctcctccgtagggtgggtctgggtgagatctgg

SEQ ID NO: 2 - DNA, 3500 bp, Homo sapien; identical to SEQ ID NO: 1

SEQ ID NO: 3 - protein, 368 aa, Homo sapien
MEGATVGEGVDLTVLAQGGRVQTSRPSGLRCRPEPSGRGRTETTGRVRVEGRGKRGGLGKAPREPEPPTPPQSRPLLKPHPAPGKAQSTPIPSRLTHRWGRARDIAALGDSAPSPVAGARSRAGPSRNPALLAMLPVSHAPRVRASSMGPGVEPRPARLAKRVAHDWLRPWPPPPRPQEGGAWVWAARRVLPPGILRASRTAVLSPGPRLPSHSARCACEGLAALGTGGAARGVRVGVREGSTQDLRTLLWGRTKHLPGAGGAPGTRRFRQLGALGICGLRPGDGLGGHAHALGLTECDRARGRAKRGGRARRRKEGVRKATAAARRRVIYTFKNQPPAPRLRRPGRVRPHARDTSVPRSLARTACHH

SEQ ID NO: 4 - protein, 193 aa, Homo sapien
MDGGTLPRSAPPAPPVPVGCAARRRPASPELLRCSRRRRPATAETGGGAAAVARRNERERNRVKLVNLGFQALRQHVPHGGASKKLSKVETLRSAVEYIRALQRLLAEHDAVRNALAGGLRPQAVRPSAPRGPPGTTPVAASPSRASSSPGRGGSSEPGSPRSAYSSDDSGCEGALSPAERELLDFSSWLGGY

SEQ ID NO: 5 - DNA, 6 bp, Artificial sequence
  oligonucleotide probe
  unsure 3-4: unknown base
canntg

SEQ ID NO: 6 - DNA, 4 bp, Artificial sequence
  sequence is synthesized
  Unsure 4: Unknown base
cgan

SEQ ID NO: 7 - DNA, 10 bp, Artificial sequence
  oligonucleotide probe
ctggccaaga

SEQ ID NO: 8 - protein, 18 aa, Artificial sequence
  polypeptide
CGRASSSPGRGGSSEPGS

SEQ ID NO: 9 - protein, 18 aa, Artificial sequence
  polypeptide
CAHDWLRPWPPPPRPQEG

SEQ ID NO: 10 - DNA, 18 bp, Artificial sequence
  oligonucleotide probe
ggcactgctgctctgcta

SEQ ID NO: 11 - DNA, 19 bp, Artificial sequence
  oligonucleotide probe
gttcacgctcccttgaaga

SEQ ID NO: 12 - DNA, 21 bp, Artificial sequence
  oligonucleotide probe
gagaagcctgtgtggggcaca

SEQ ID NO: 13 - DNA, 20 bp, Artificial sequence
  oligonucleotide probe
aggaacccagctttgttagc

SEQ ID NO: 14 - DNA, 18 bp, Artificial sequence
  oligonucleotide probe
agggtctcagccaatcgt

SEQ ID NO: 15 - DNA, 21 bp, Artificial sequence
  oligonucleotide probe
cgtacgcgcttcctcaatggg

SEQ ID NO: 16 - DNA, 19 bp, Artificial sequence
  oligonucleotide probe
aaggcgcgctgagtcctgc

SEQ ID NO: 17 - DNA, 23 bp, Artificial sequence
  oligonucleotide probe
ttacctcataggtcgagggcgct

SEQ ID NO: 18 - DNA, 24 bp, Artificial sequence
  oligonucleotide probe
cgagctactcgacttctccagctg

SEQ ID NO: 19 - DNA, 20 bp, Artificial sequence
  oligonucleotide probe
gcggattctcatggaacaca

SEQ ID NO: 20 - DNA, 20 bp, Artificial sequence
  oligonucleotide probe
ggtcagccaggagcttcttg

SEQ ID NO: 21 - DNA, 25 bp, Artificial sequence
  oligonucleotide probe
cacaagctgaaggcagacaaggccc

SEQ ID NO: 22 - DNA, 18 bp, Artificial sequence
  oligonucleotide probe
caccgacgggaagcgaga

SEQ ID NO: 23 - DNA, 21 bp, Artificial sequence
  oligonucleotide probe
cctagtggtggcaggccgtac

SEQ ID NO: 24 - DNA, 21 bp, Artificial sequence
  oligonucleotide probe
ggctggaggtgggggatactg

SEQ ID NO: 25 - DNA, 19 bp, Artificial sequence
  oligonucleotide probe
ctggagcccggggatagga

SEQ ID NO: 26 - DNA, 22 bp, Artificial sequence
  oligonucleotide probe
cgggctccagacgacctaggac

SEQ ID NO: 27 - DNA, 24 bp, Artificial sequence
  oligonucleotide probe
ctcataggtcgagggcgctcagta

SEQ ID NO: 28 - DNA, 34 bp, Artificial sequence
  oligonucleotide probe
aaagggaaactcgagatggacggcggcacactgc

SEQ ID NO: 29 - DNA, 45 bp, Artificial sequence
  oligonucleotide probe
aaagggaaaaagcttgtagccccctaaccagctggagaagtcgag

SEQ ID NO: 30 - DNA, 35 bp, Artificial sequence
  oligonucleotide probe
aaagggaaactcgagatggagggagccacggtggg

SEQ ID NO: 31 - DNA, 35 bp, Artificial sequence
  oligonucleotide probe
aaagggaaaaagcttgtggtggcaggccgtacgcg

SEQ ID NO: 32 - DNA, 17 bp, Artificial sequence
  oligonucleotide probe
ccccacagcttctcgac

SEQ ID NO: 33 - DNA, 21 bp, Artificial sequence
  oligonucleotide probe
agcagggtttgaggagtagtg

SEQ ID NO: 34 - DNA, 47 bp, Artificial sequence
  oligonucleotide probe
ggattctaatacgactcactatagggcctggcaaacggagacctatt

SEQ ID NO: 35 - DNA, 48 bp, Artificial sequence
  oligonucleotide probe
ctatgaaattaaccctcactaaagggaaggagtagtgaccacggaagt

SEQ ID NO: 36 - DNA, 21 bp, Artificial sequence
  oligonucleotide probe
cgccccgggaacctgaacctc

SEQ ID NO: 37 - DNA, 19 bp, Artificial sequence
  oligonucleotide probe
gccgccgtcgcctttctca

SEQ ID NO: 38 - DNA, 48 bp, Artificial sequence
  oligonucleotide probe
ggattctaatacgactcactatagggcatcttgcgcgcctcccgaaca

SEQ ID NO: 39 - DNA, 47 bp, Artificial sequence
  oligonucleotide probe
ctatgaaattaaccctcactaaagggacgccgccgtcgcctttctca

SEQ ID NO: 40 - DNA, 17 bp, Artificial sequence
  oligonucleotide probe
gccgggacctgactgac

SEQ ID NO: 41 - DNA, 21 bp, Artificial sequence
  oligonucleotide probe
aacaaataaagccatgccaat

SEQ ID NO: 42 - DNA, 46 bp, Artificial sequence
  oligonucleotide probe
ggattctaatacgactcactatagggcgctgcctgacggccaggtc

SEQ ID NO: 43 - DNA, 49 bp, Artificial sequence
  oligonucleotide probe
ctatgaaattaaccctcactaaagggagagtacttgcgctcaggaggag

SEQ ID NO: 44 - DNA, 18 bp, Artificial sequence
  oligonucleotide probe
gctattaccatggtgatg

SEQ ID NO: 45 - DNA, 17 bp, Artificial sequence
  oligonucleotide probe
ccttgaagaagatggtg

SEQ ID NO: 46 - DNA, 21 bp, Artificial sequence
  oligonucleotide probe
gtgaaatttgtgatgctattg

SEQ ID NO: 47 - DNA, 18 bp, Artificial sequence
  oligonucleotide probe
tgcctttctctccacagg

SEQ ID NO: 48 - DNA, 17 bp, Artificial sequence
  oligonucleotide probe
gcaagaagctgagcaag

SEQ ID NO: 49 - DNA, 19 bp, Artificial sequence
  oglionucleotide probe
ggactcagatctcgagatg

SEQ ID NO: 50 - DNA, 19 bp, Artificial sequence
  oglionucleotide probe
cgtgaagctggtgaacttg

SEQ ID NO: 51 - DNA, 17 bp, Artificial sequence
  oglionucleotide probe
ccttgctcagcttcttg

SEQ ID NO: 52 - DNA, 20 bp, Artificial sequence
  oglionucleotide probe
cctaaccagctggagaagtc

SEQ ID NO: 53 - DNA, 18 bp, Artificial sequence
  oglionucleotide probe
cctgcgtaccttgctttg

SEQ ID NO: 54 - DNA, 19 bp, Artificial sequence
  oglionucleotide probe
cctcagtctcgaccactcc

SEQ ID NO: 55 - DNA, 20 bp, Artificial sequence
  oglionucleotide probe
ccacctgtgcgttaatctac

SEQ ID NO: 56 - DNA, 17 bp, Artificial sequence
  oglionucleotide probe
cctccttccttcgcctc

SEQ ID NO: 57 - DNA, 19 bp, Artificial sequence
  oglionucleotide probe
cgtaggtcagggtggtcac